(19) United States
(12) Patent Application Publication
(10) Pub. No.: US 2012/0083018 A1
(43) Pub. Date: Apr. 5, 2012

(54) THERMOSTABLE DNA POLYMERASES AND METHODS OF USE

(76) Inventors: Thomas W. Schoenfeld, Madison, WI (US); David A. Mead, Middleton, WI (US)

(21) Appl. No.: 13/313,783

(22) Filed: Dec. 7, 2011

Related U.S. Application Data
(63) Continuation-in-part of application No. 12/761,175, filed on Apr. 15, 2010.
Application No. 12/761,175 is a continuation-in-part of application No. 12/089,221, filed on Apr. 4, 2008, now Pat. No. 8,093,030.
(60) Provisional application No. 61/169,470, filed on Apr. 15, 2009.
(60) Provisional application No. 60/805,695, filed on Jun. 23, 2006.
(60) Provisional application No. 60/724,207, filed on Oct. 6, 2005.

(51) Int. Cl. C12P 19/34; C12N 9/12

(52) U.S. Cl. 435/91.51; 435/194; 435/91.5

(57) ABSTRACT

Thermostable viral and microbial polymerases exhibiting a combination of activities selected from proofreading (3′-5′) exonuclease activity, nick translating (5′-3′) nuclease activity, synthetic primer-initiated polymerase activity, nick-initiated polymerase activity, reverse transcriptase activity, strand displacement activity, terminal transferase activity, primase activity, and/or efficient incorporation of chain terminating analogs. Some of the polymerases provided herein include a first motif and a second motif. The first motif preferably has the sequence X₁X₂X₃DX₄PX₅IELRX₆X₇X₈, wherein X₁ is I or V; X₄ is F or Y; X₈ is G or A; and X₂, X₃, X₅, X₆, and X₇ are any amino acid. The second motif preferably has the sequence RX₉X₁₀X₁₁KSANX₁₂GX₁₃X₁₄YG, wherein X₁₁ is G or A; X₁₂ is F, L, or Y; X₁₃ is L or V; X₁₄ is I or L; and X₉ and X₁₀ are any amino acid. Also provided are reagents for expressing the polymerases, polynucleotides encoding the polymerases, host cells expressing the polymerases, and methods of using the polymerases.

### CROSS-REFERENCE TO RELATED APPLICATIONS

This application is a continuation-in-part under 35 U.S.C. §120 of co-pending U.S. patent application Ser. No. 12/761,175, filed Apr. 15, 2010, which claims priority under 35 U.S.C. §119(e) to U.S. Provisional Patent Application 61/169,470, filed Apr. 15, 2009, and is a continuation-in-part under 35 U.S.C. §120 of U.S. patent application Ser. No. 12/089,221, filed as PCT/US06/39406 on Oct. 6, 2006 and entering the U.S. national stage under U.S.C. §371 on Apr. 4, 2008, which claims priority under 35 U.S.C. §119(e) to U.S. Provisional Patent Application 60/805,695, filed Jun. 23, 2006, and U.S. Provisional Patent Application 60/724,207, filed Oct. 6, 2005, all of which are incorporated herein by reference in their entirety.

### STATEMENT REGARDING FEDERALLY SPONSORED RESEARCH

This invention was made with United States government support awarded by the National Science Foundation (Grant Nos. 0109756 and 0215988) and the National Institutes of Health (Grant Nos. R43 HG002714-01 and 1R43HG004095-01). The United States government has certain rights in this invention.

### REFERENCE TO SEQUENCE LISTING

This application includes a sequence listing submitted herewith. The content of the sequence listing is incorporated herein by reference in its entirety.

### FIELD OF THE INVENTION

The present invention is directed to thermostable DNA polymerases and methods of use thereof. More specifically, the present invention is directed to virally or microbially derived DNA polymerases, variants thereof, and methods of using the DNA polymerases.

### BACKGROUND

There are seven recognized families of DNA polymerases, including A, B, C, D, X, Y, and RT. The most widely used DNA polymerase reagents are family A and B polymerases, especially those that are stable to greater than 90° C. and are active at temperatures of at least 70° C. These DNA polymerases, referred to as “thermostable” DNA polymerases, are commonly used in DNA detection and analysis methods employing such high temperatures, e.g., polymerase chain reaction and thermocycled DNA sequencing.

Thermostable DNA polymerases are commonly used in recombinant DNA technology to generate polynucleotide sequences from both known and unknown target sequences. It is appreciated that the biochemical attributes of a given enzyme may either enhance or limit its usefulness, depending upon the particular reaction conditions and desired functions. Characteristics that are generally considered to affect the utility of thermostable polymerases include strand displacement activity, processivity, both 3′-5′ and 5′-3′ exonuclease activity, affinity for template DNA and for nucleotides (both canonical and modified), error rate and degree of thermostability. Despite extensive investigation to discover new polymerases and attempts to manipulate buffer formulations to optimize polymerase activity, there remains a need for thermostable DNA polymerases having an appropriate combination of the above attributes for particular applications.

Many bacterial and archaeal thermostable DNA polymerases are known and used, including Taq, Vent, and Bst. Each of these enzymes, while effective for use in particular applications, has limitations. For example, both Bst and Taq lack proofreading activity and, therefore, have a relatively high error rate. Extensive efforts to isolate new thermostable DNA polymerases have provided dozens of alternative enzymes, but only modest improvements in biochemical properties have resulted.

Viral DNA polymerases (including phage polymerases), like their bacterial counterparts, catalyze template-dependent synthesis of DNA. However, viral polymerases differ significantly in their biochemical characteristics from the bacterial polymerases currently used for most DNA and RNA analysis. For example, T5, T7, and phi29 DNA polymerases are among the most processive enzymes known. RB49 DNA polymerase, in addition to having a highly active proofreading function, has the highest known fidelity of initial incorporation. T7 and phi29 DNA polymerases have the lowest measured replication slippage due to high processivity. T7 DNA polymerase can efficiently incorporate dideoxynucleotides, thereby enabling facile chain terminating DNA sequence analysis. The viral reverse transcriptases are unique among reagents in their efficiency in synthesizing a DNA product using an RNA template.

Despite their advantages, deficiencies among the available DNA polymerase enzymes are apparent. Notably, there is no thermostable viral polymerase widely available. U.S. Patent Publication 2003/0087392 describes a moderately thermostable polymerase isolated from bacteriophage RM378. Although this polymerase is described as “expected to be much more thermostable than [that] of bacteriophage T4,” and is said to lack both 3′-5′ and 5′-3′ exonuclease activities, RM378 polymerase is not thermostable enough for thermocycled amplification or sequencing. A larger pool of potential viral and microbial reagent DNA polymerases is needed for use in DNA detection and analysis methods.

### SUMMARY OF THE INVENTION

The invention pertains generally to polymerases suitable for use as reagent enzymes. Because the polymerases described herein were derived from thermophilic viruses and microbes, they are significantly more thermostable than those of other (e.g. mesophilic) viruses and microbes, such as the T4 bacteriophage of Escherichia coli or E. coli, itself. The enhanced stability of the polymerases described herein permits their use under temperature conditions which would be prohibitive for other enzymes, thereby increasing the range of conditions which can be employed, allowing thermocycling and improving amplification specificity of isothermal methods.

One aspect of the invention provides a substantially purified DNA polymerase comprising an amino acid sequence having a motif selected from the group consisting of a first motif and a second motif. The first motif preferably has the sequence X₁X₂X₃DX₄PX₅IELRX₆X₇X₈, wherein X₁ is I or V; X₄ is F or Y; X₈ is G or A; and X₂, X₃, X₅, X₆, and X₇ are any amino acid. The second motif preferably has the sequence RX₉X₁₀X₁₁KSANX₁₂GX₁₃X₁₄YG, wherein X₁₁ is G or A; X₁₂ is F, L, or Y; X₁₃ is L or V; X₁₄ is I or L; and X₉ and X₁₀ are any amino acid. Exemplary, non-limiting motifs comprise sequences ITADFPQIELRLAG, VIADYPQIELRLAG, RQIGKSANFGLIYG, RQIGKSANLGLIYG, RQIGKSANYGLIYG, and RQVAKSANFGLIYG.

Another aspect of the invention provides a substantially purified polymerase having an amino acid sequence comprising SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:25, SEQ ID NO:26, SEQ ID NO:27, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:61, SEQ ID NO:63, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, or sequence variants thereof.

One aspect of the invention also provides a substantially purified polymerase that demonstrates nick-initiated polymerase activity, primer-initiated polymerase activity, 3′-5′ exonuclease (proofreading) activity, reverse transcriptase activity and/or strand displacement activity. In some embodiments of the invention, the purified polymerases lack 3′-5′ exonuclease activity. Other polymerases of the invention do not discriminate against nucleotide analog incorporation.

Other aspects of the invention provide isolated polynucleotides encoding the polymerases, polynucleotide constructs comprising the polynucleotides, host cells comprising the polynucleotide constructs, and methods of producing thermostable polymerases.

In another aspect, the invention provides a method of synthesizing a DNA copy or complement of a polynucleotide template. The method includes contacting the template with a polypeptide of the invention under conditions sufficient to promote synthesis of the copy or complement. In some embodiments, the template is RNA, and in other embodiments, the template is DNA. In yet other embodiments, the template comprises an RNA template and a DNA template; the copy or complement comprises a first DNA copy or complement and a second DNA copy or complement, wherein the first DNA copy or complement is the DNA template; the polymerase synthesizes the first DNA copy or complement from the RNA template; and the polymerase synthesizes the second DNA copy from the DNA template.

Other aspects of the invention will become apparent by consideration of the detailed description of several embodiments and from the claims.

### BRIEF DESCRIPTION OF THE DRAWINGS

FIG. 1 is a photographic image of an electrophoretic gel showing results of polymerase chain reaction (PCR) amplification of a 1 kb pUC19 sequence using a polymerase of the invention and two commercially available polymerases.

FIG. 2 is a photographic image of an electrophoretic gel showing the results of PCR amplification using a polymerase of the invention.

FIG. 3 is a photographic image of an electrophoretic gel showing results of PCR amplification of a 1 kb Bacillus cyc gene sequence (a guanidine/cytosine-rich template) using a polymerase of the invention and five commercially available polymerases.

FIG. 4 is a photographic image of an electrophoretic gel used to resolve the product of an RT-PCR reaction in which a 294 bp cDNA was reverse-transcribed and amplified from total mouse RNA using specific primers and a polymerase of the invention.

FIG. 5A shows a photographic image of an electrophoretic gel used to resolve an isothermal amplification reaction in which single-stranded and double-stranded templates were amplified using a polymerase of the invention.

FIG. 5B shows a photographic image of an electrophoretic gel used to resolve a PCR amplification reaction to verify the identity of the isothermal amplification product shown in FIG. 5A.

FIG. 6 is a photographic image of an electrophoretic gel used to resolve amplification reactions carried out without added primers using two polymerases of the invention in the presence or absence of a commercially available nicking enzyme.

FIGS. 7A-7D show a sequence alignment of a family of eight sequences isolated from Great Boiling Spring (Gerlach, Nev.) in a functional screen of a thermophilic clone library showing a minimum of 97% sequence identity to one another over at least a portion of their respective sequences. FIGS. 7B-7D show continuations of the same sequences shown in FIG. 7A. Motifs A and B are highlighted.

FIGS. 8A-8I show a sequence alignment of viral polymerases isolated from Octopus Hot Spring (Yellowstone National Park), Great Boiling Spring (Gerlach Nev.), and Little Hot Creek (Long Valley, Calif.). FIGS. 8B-8I show continuations of the same sequences shown in FIG. 8A. Motifs A and B are highlighted.

FIG. 9A depicts a sequence alignment of Motif A variations, including those of the present invention.

FIG. 9B depicts a sequence alignment of Motif B variations, including those of the present invention.

FIG. 10 is a photographic image of an electrophoretic gel showing results of polymerase chain reaction (PCR) amplification of a 10 kb of sequence of phage lambda (GenBank Accession No. NC_{—}001416) using a polymerase of the invention (Dtu polymerase) and primers of SEQ ID NOS. 58 and 59. Lane 1 shows a molecular weight marker ranging from 250 to 10,000 base pairs. Lane 2 shows the amplification product. The arrow indicates the location of the expected amplification product.

FIG. 11 is a photographic image of an electrophoretic gel showing the temperature profile of D. turgidum DNA polymerase versus Taq DNA polymerase.

FIG. 12 is a photographic image of an electrophoretic gel showing the reduced mispriming of D. turgidum DNA polymerase versus Taq DNA polymerase.

### DETAILED DESCRIPTION OF THE INVENTION

Before any embodiments of the invention are explained in detail, it is to be understood that the invention is not limited in its application to the details of construction and the arrangement of components set forth in the following description or illustrated in the following figures and examples. The invention is capable of other embodiments and of being practiced or of being carried out in various ways. Also, it is to be understood that the phraseology and terminology used herein is for the purpose of description and should not be regarded as limiting. The terms “including,” “comprising,” or “having” and variations thereof are meant to encompass the items listed thereafter and equivalents thereof as well as additional items.

Any version of any component or method step of the invention may be used with any other component or method step of the invention. The elements described herein can be used in any combination whether explicitly described or not.

All combinations of method steps as used herein can be performed in any order, unless otherwise specified or clearly implied to the contrary by the context in which the referenced combination is made.

All patents, patent publications, and peer-reviewed publications (i.e., “references”) cited herein are expressly incorporated herein by reference in their entirety to the same extent as if each individual reference were specifically and individually indicated as being incorporated by reference. In case of conflict between the present disclosure and the incorporated references, the present disclosure controls.

As used in this specification and the appended claims, the singular forms “a,” “an,” and “the” include plural referents unless the content clearly dictates otherwise. Thus, for example, reference to a composition containing “a polynucleotide” includes a mixture of two or more polynucleotides. It should also be noted that the term “or” is generally employed in its sense including “and/or” unless the content clearly dictates otherwise. All publications, patents and patent applications referenced in this specification are indicative of the level of ordinary skill in the art to which this invention pertains. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

It also is specifically understood that any numerical value recited herein includes all values from the lower value to the upper value, i.e., all possible combinations of numerical values between the lowest value and the highest value enumerated are to be considered to be expressly stated in this application. For example, if a concentration range is stated as 1% to 50%, it is intended that values such as 2% to 40%, 10% to 30%, or 1% to 3%, etc., are expressly enumerated in this specification. These are only examples of what is specifically intended.

The invention relates to polymerases, polynucleotides, and reagents encoding the polymerases and methods for using the polymerases. The polymerases of the invention are suitable for sequence-specific methods including PCR, as well as whole-genome nucleic acid amplification. As will be appreciated, the polymerases described herein are useful in any research or commercial context wherein polymerases typically are used for DNA analysis, detection, or amplification.

As used herein, “polymerase” refers to an enzyme with polymerase activity that may or may not demonstrate further activities, including, but not limited to, nick-initiated polymerase activity, primer-initiated polymerase activity, 3′-5′ exonuclease (proofreading) activity, reverse transcriptase activity, terminal transferase, primase, and/or strand displacement activity. Polymerases of the invention suitably exhibit one or more activities selected from polymerase activity, proofreading (3′-5′) exonuclease activity, nick translating (5′-3′) nuclease activity, primer-initiated polymerase activity, reverse transcriptase activity, strand displacement activity, and/or increased propensity to incorporate chain terminating analogs. As will be appreciated by the skilled artisan, an appropriate polymerase may be selected from those described herein based on any of these and other activities or combinations thereof, depending on the application of interest.

The polymerases described herein are of viral and microbial origin. For purposes of this description, a “virus” is a nucleoprotein entity which depends on host cells for the production of progeny. The term encompasses viruses that infect eukaryotic, bacterial or archaeal hosts, and may be used interchangeably with “bacteriophage,” “archaeaphage,” or “phage,” depending on the host. A “microbe” encompasses any microscopic bacterial, archaeal, or eukaryotic cell.

The purified polymerases of the invention were compared to known polymerases and found to have one or more enzymatic domains conserved, or were shown to have DNA polymerase activity. The enzymatic domains and other domains (e.g., signal peptide, linker domains, Motif A, Motif B etc.) can be readily identified by analysis and comparison of the sequence of the viral polymerases with sequences of other polymerases using publicly available comparison programs, such as ClustalW (European Bioinformatics Institute, Hinxton, England).

The polymerases of the invention are substantially purified polypeptides. As used herein, the term “purified” refers to material that is at least partially separated from components which normally accompany it in its native state. Purity of polypeptides is typically determined using analytical techniques such as polyacrylamide gel electrophoresis or high performance liquid chromatography. A polypeptide that is the predominant species present in a preparation is “substantially purified.” The term “purified” denotes that a preparation containing the polypeptide may give rise to essentially one band in an electrophoretic gel. Suitably, polymerases of the invention are at least about 85% pure, more suitably at least about 95% pure, and most suitably at least about 99% pure.

The polymerases of the invention are thermostable. The term “thermostable” is used herein to refer to a polymerase that retains at least a portion of one activity after incubation at relatively high temperatures, i.e., 50-100° C. In some cases, thermostable enzymes exhibit optimal activity at relatively high temperatures, i.e., about 50-100° C. In some embodiments, the thermostable polymerases exhibit optimal activity from about 60° C. to 70° C. Most suitably, thermostable enzymes are capable of maintaining at least a portion of at least one activity after repeated exposure to temperatures from about 90° C. to about 98° C. for up to several minutes for each exposure.

The polypeptides comprising the polymerases of the invention may comprise about 400-1500 residues, more preferably about 450-1000 residues, and most preferably about 480-800 residues.

The polymerases of the invention may be about 44-165 kDa, more preferably about 50-110 kDa, and most preferably about 53-90 kDa. In some specific versions, the polymerase is about 55 kDa.

The polymerases of the invention have amino acid sequences comprising SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:25, SEQ ID NO:26, SEQ ID NO:27, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:61, SEQ ID NO:63, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, or sequence variants thereof, i.e., variants of any of the previously listed sequences.

The term “sequence variants” refers to polymerases that retain at least one activity and have at least about 80% identity, more suitably at least about 85% identity, more suitably at least about 90% identity, more suitably at least about 95% identity, and most suitably at least about 98% or 99% identity, to the amino acid sequences provided. Percent identity may be determined using the algorithm of Karlin and Altschul (Proc. Natl. Acad. Sci. 87: 2264-68 (1990), modified Proc. Natl. Acad. Sci. 90: 5873-77 (1993). Such algorithm is incorporated into the BLASTx program, which may be used to obtain amino acid sequences homologous to a reference polypeptide, as is known in the art.

The term “sequence variants” may also be used to refer to proteins having amino acid sequences including conservative amino acid substitutions, unless explicitly stated otherwise. “Conservative amino acid substitution” or variants thereof refers to the replacement of one amino acid by an amino acid having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, phenylalanine, methionine, tryptophan), beta-branched side chains (e.g., threonine, valine, isoleucine), and aromatic side chains (e.g., tyrosine, phenylalanine, tryptophan, histidine).

The term “sequence variants” also refers to proteins that are subjected to site-directed mutagenesis wherein one or more substitutions, additions or deletions may be introduced, e.g., as described below, to provide altered functionality, as desired.

The term “sequence variants” also refers to homologs. Homologs can be identified by homologous nucleic acid and polypeptide sequence analyses. Known nucleic acid and polypeptide sequences in one organism can be used to identify homologous polypeptides in another organism. For example, performing a query on a database of nucleic acid or polypeptide sequences can identify homologs thereof. Homologous sequence analysis can involve BLAST or PSI-BLAST analysis of databases using known polypeptide amino acid sequences (see, e.g., Altschul et al., 1990). Those proteins in the database that have greater than 35% sequence identity are candidates for further evaluation for suitability in the systems and methods of the invention. If desired, manual inspection of such candidates can be carried out in order to narrow the number of candidates that can be further evaluated. Manual inspection is performed by selecting those candidates that appear to have conserved domains. Determining nucleic acid sequences from discovered homologous amino acid sequences or amino acid sequences from discovered homologous nucleic acid sequences can be deduced using the genetic code.

The term “sequence variants,” used in references to nucleotide coding sequences, refers to degenerate sequences that encode the same polypeptides as disclosed herein. Such degenerate variants can be deduced with the genetic code.

The term “sequence variants” also refers to fragments of the sequences described herein. “Fragment” means a portion of the full length sequence. For example, a fragment of a given polypeptide is at least one amino acid fewer in length than the full length polypeptide (e.g. one or more internal or terminal amino acid deletions from either amino or carboxy termini). Fragments therefore can be any length up to, but not including, the full length polypeptide. Suitable fragments of the polypeptides described herein include but are not limited to those having 50%, 60%, 70%, 75%, 80%, 85%, 90%, 95%, 96%, 97%, 98%, or more of the length of the full length polypeptide.

The term “sequence variants” also refers to repeating units of the sequences described herein. “Repeating units” means a repetition of a given sequence in tandem. Also included are polypeptides having repeating units of fragments of the sequences described herein.

Suitable variants of the nucleic acid or polypeptide sequences disclosed herein have the same type of activity (without regard to the degree of the activity) as the nucleic acid or polypeptide to which the sequence corresponds. Such activities may be tested according to the assays described in the Examples below and according to methods known in the art.

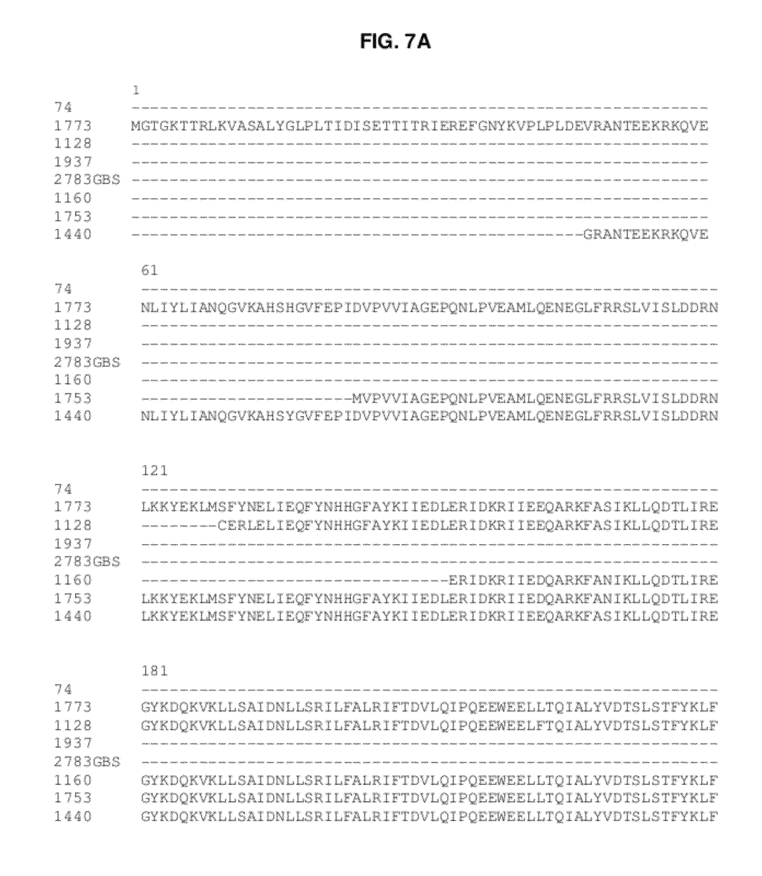

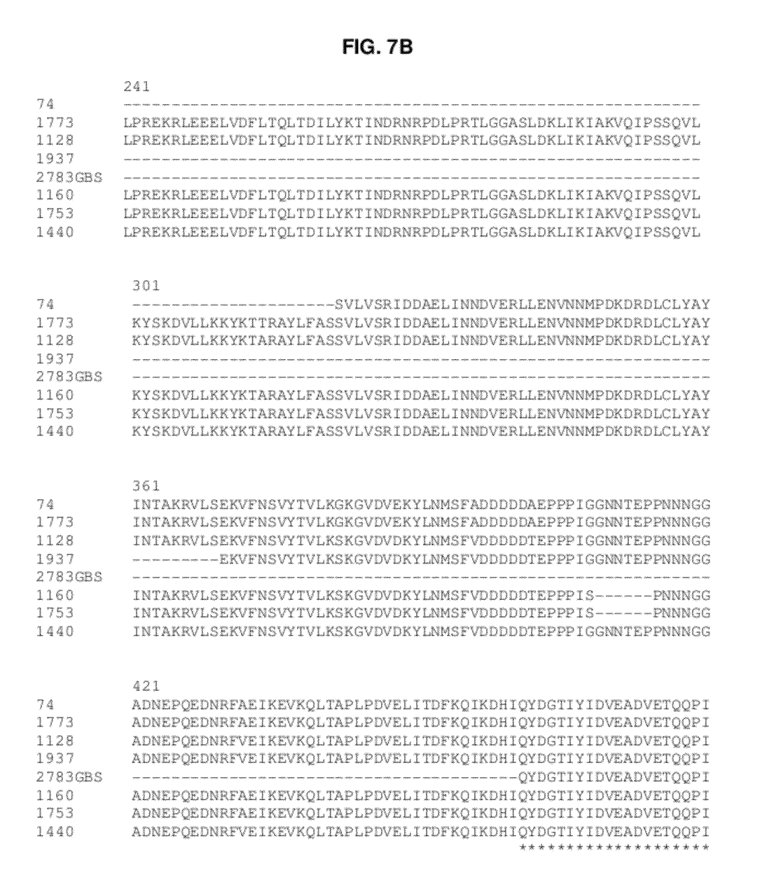

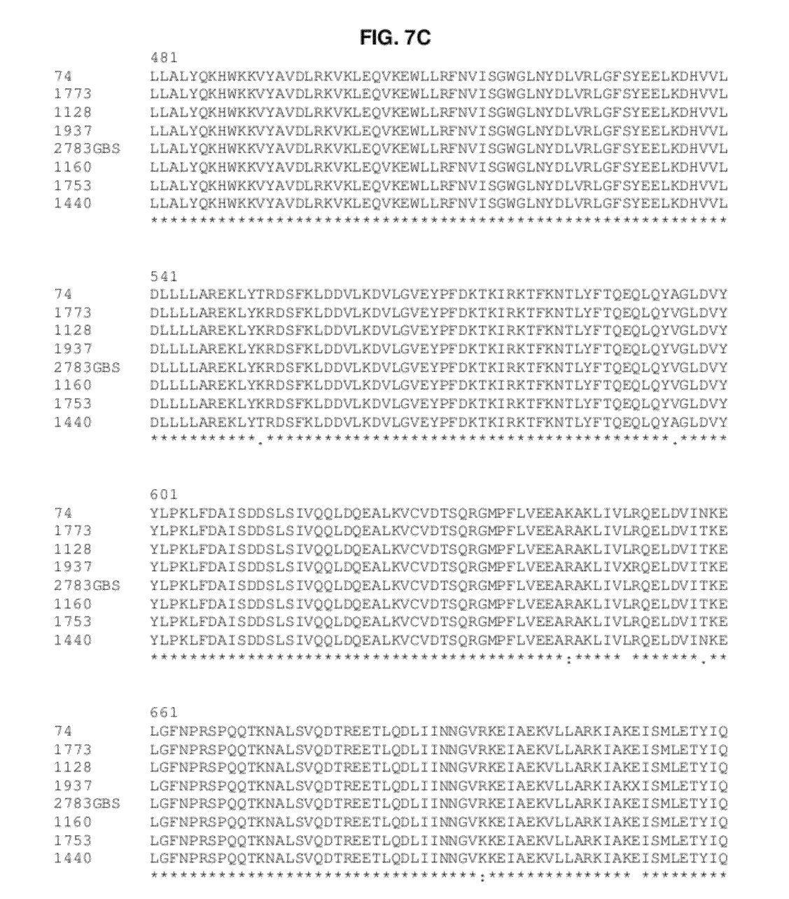

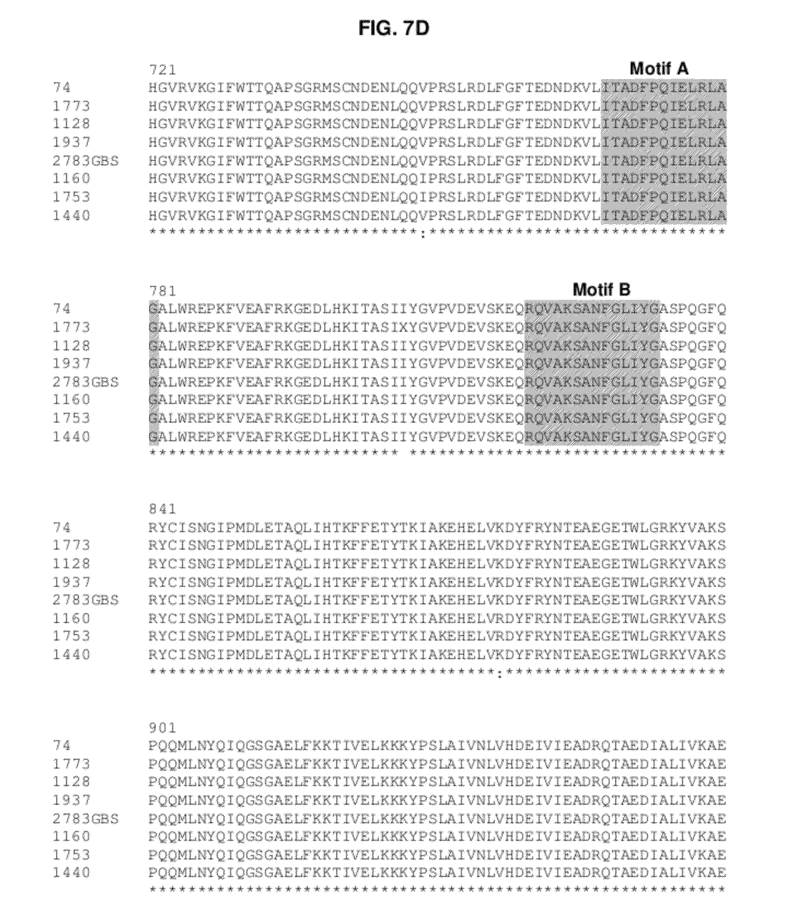

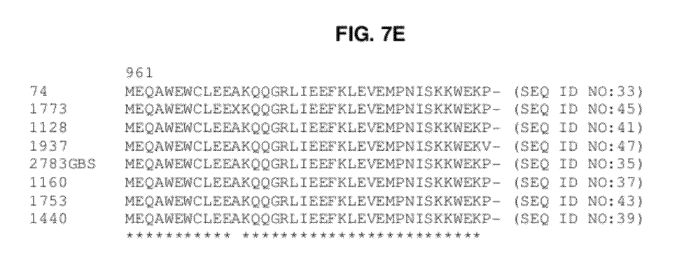

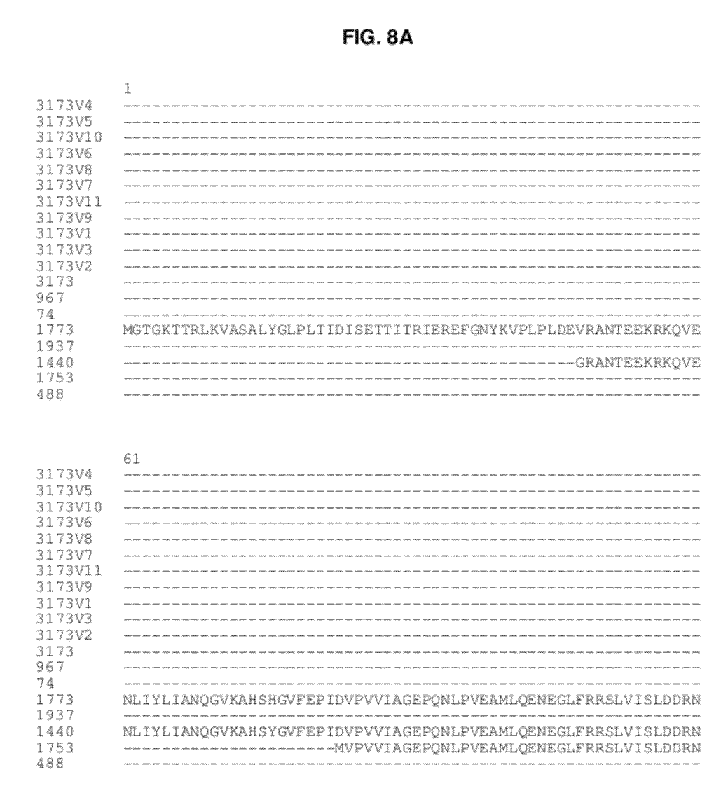

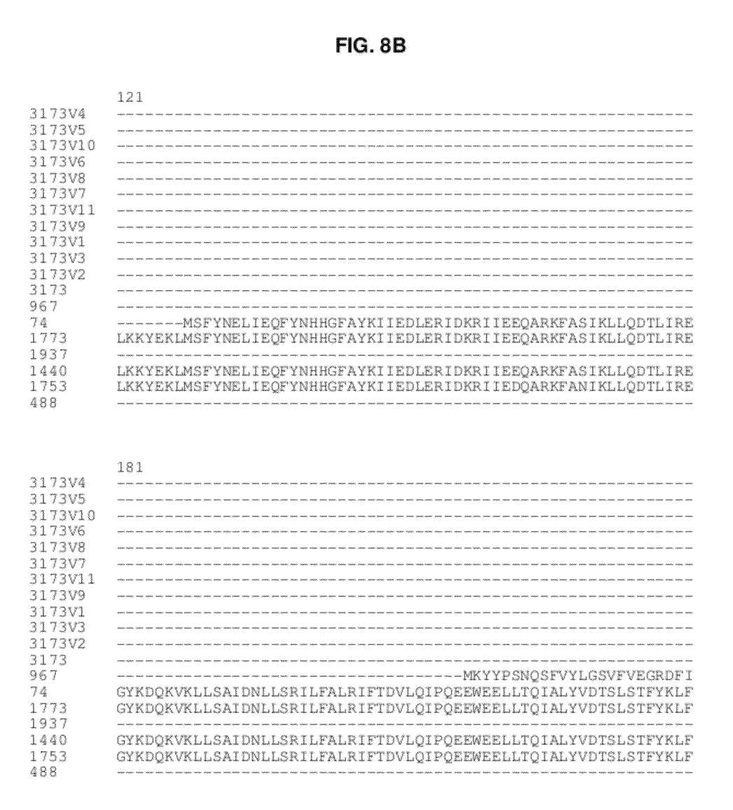

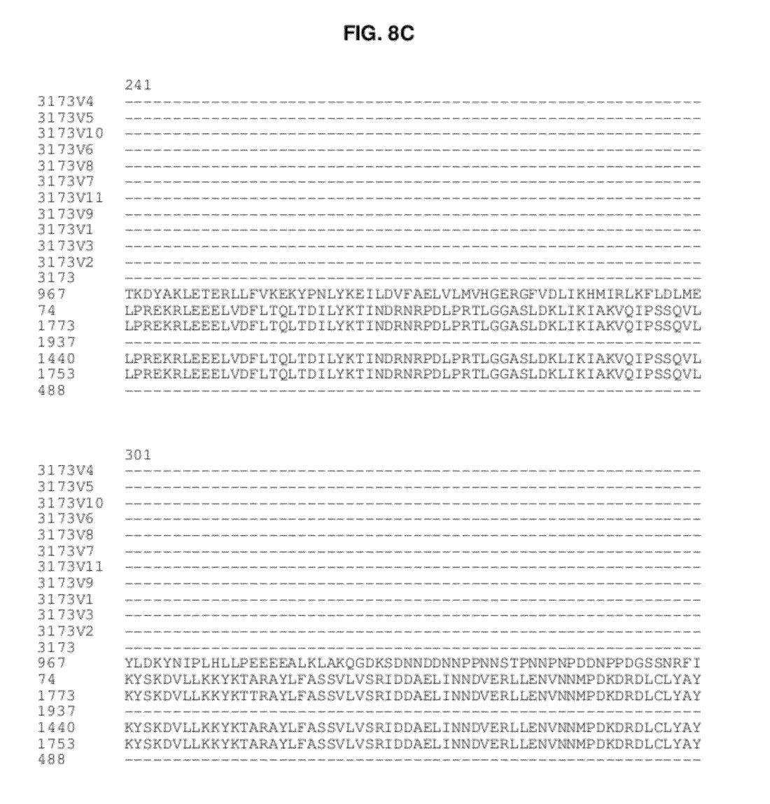

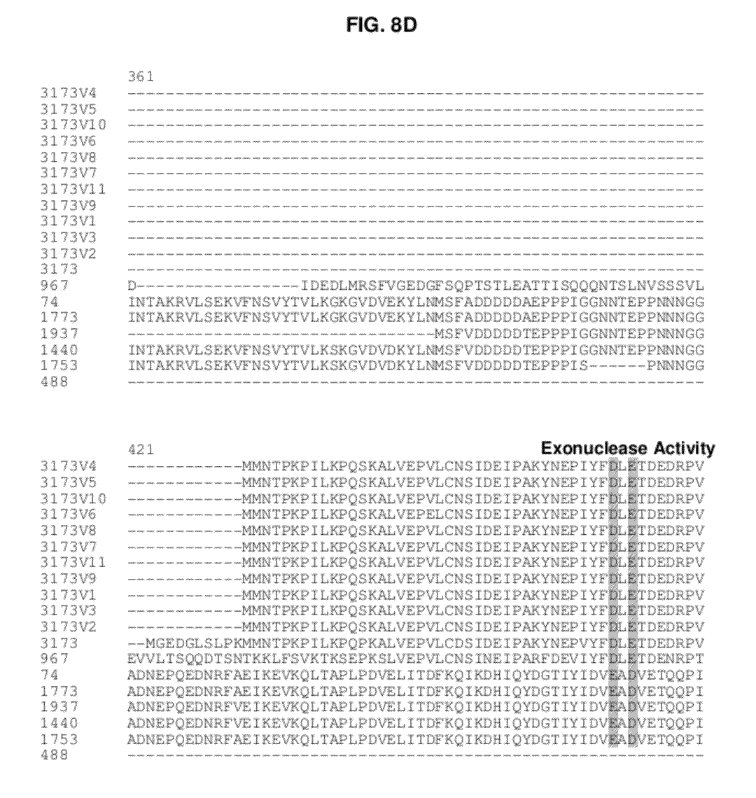

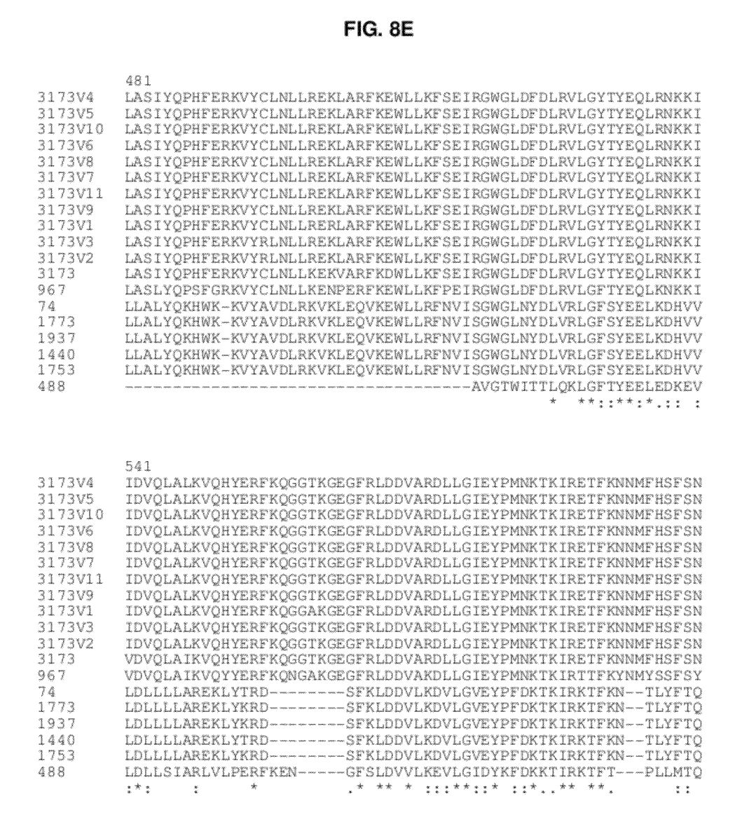

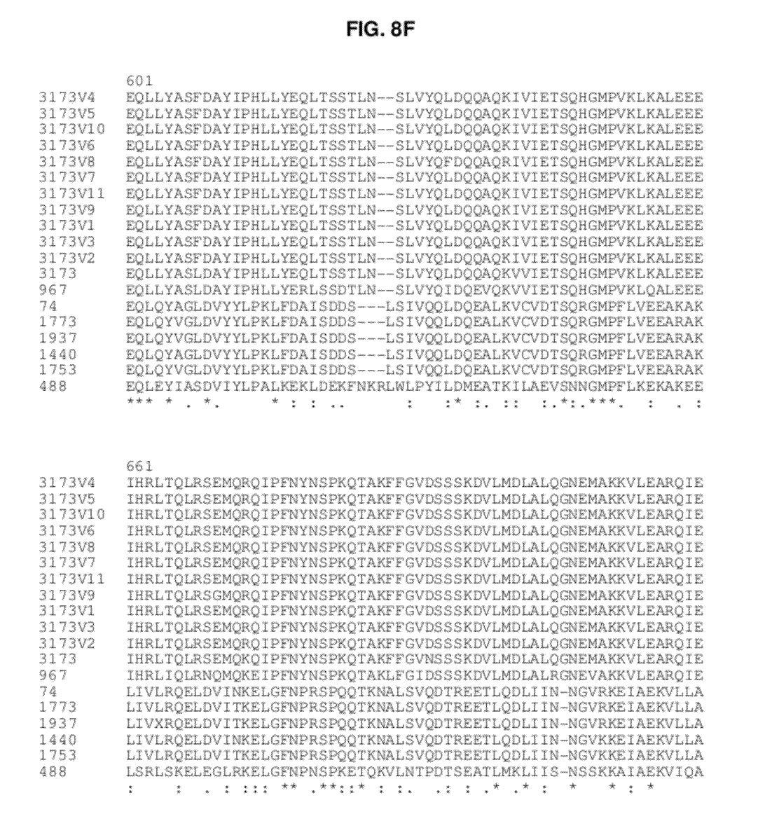

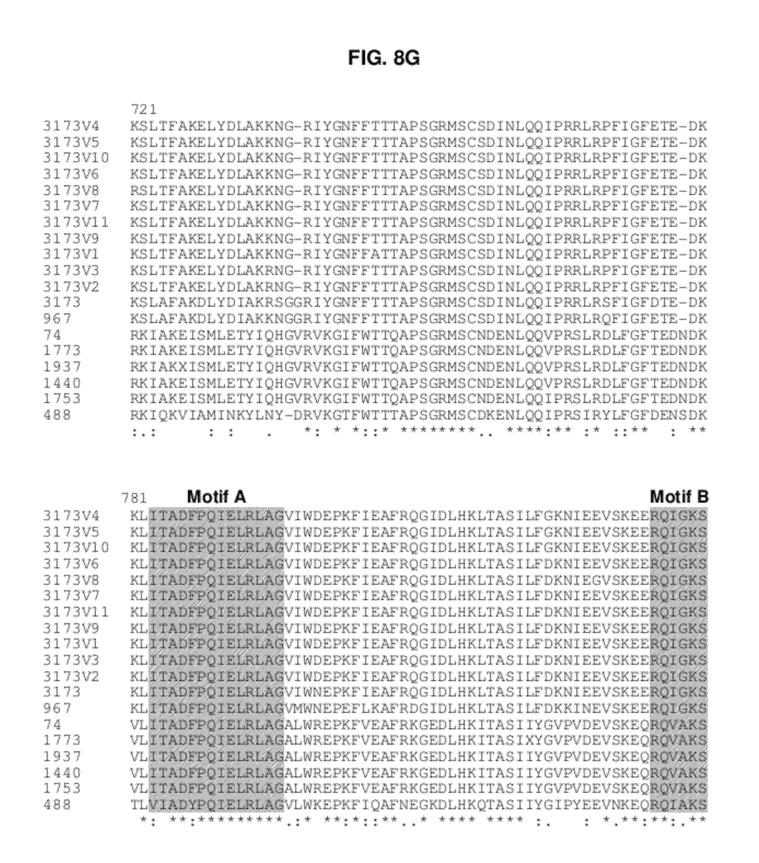

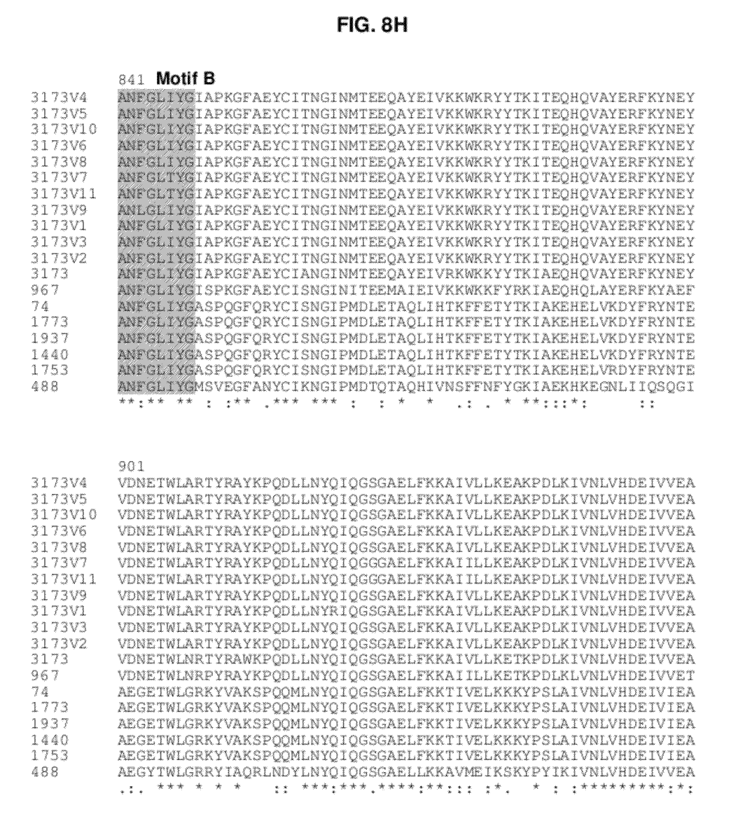

Viral polymerases of the present invention can be defined by the presence of one or both of two motifs. A first of the two motifs has sequence X₁X₂X₃DX₄PX₅IELRX₆X₇X₈, wherein X₁ is I or V; X₄ is F or Y; X₈ is G or A; and X₂, X₃, X₅, X₆, and X₇ are any amino acid (SEQ ID NO:81). Any specific sub-combinations of the motif as defined SEQ ID NO:81 are expressly included in the invention. Non-limiting examples of such a motif can be found as shown as “Motif A” in FIGS. 7D, 8G, and 8H, and include, for example, sequences ITADFPQIELRLAG (residues 358-371 of SEQ ID NO:6) and VIADYPQIELRLAG (residues 257-270 of SEQ ID NO:4).

A second of the two motifs has the sequence RX₉X₁₀X₁₁KSANX₁₂GX₁₃X₁₄YG, wherein X₁₁ is G or A; X₁₂ is F, L, or Y; X₁₃ is L or V; X₁₄ is I or L; and X₉ and X₁₀ are any amino acid (SEQ ID NO:85). Any specific sub-combinations of the motif as defined in SEQ ID NO:85 are expressly included in the invention. Non-limiting examples of such a motif can be found as shown as “Motif B” in FIGS. 7D, 8G, and 8H, and include, for example, sequences RQIGKSANFGLIYG (residues 410-423 of SEQ ID NO:6), RQIGKSANLGLIYG (residues 399-412 of SEQ ID NO:75), RQIGKSANYGLIYG (residues 410-423 of SEQ ID NO:26), and RQVAKSANFGLIYG (residues 773-786 of SEQ ID NO:33).

Exemplary polypeptides comprising the motifs as defined by SEQ ID NO:81 and SEQ ID NO: 85 include SEQ ID NO:4, SEQ ID NO:6, SEQ ID NO:14, SEQ ID NO:25, SEQ ID NO:26, SEQ ID NO:27, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:61, SEQ ID NO:63, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, and sequence variants thereof.

In one particularly suitable embodiment, a polymerase of the invention includes the sequence of amino acids shown in SEQ ID NO:6. This polymerase is also referred to herein as “polymerase 3173.” In other embodiments, polymerases of the invention include mutated forms of polymerase 3173, including those having sequences shown in SEQ ID NOS:25-27. The mutated forms of polymerase 3173 suitably exhibit strand displacement activity, substantially reduced exonuclease activity, reduced discrimination for nucleotide analogs, or combinations thereof, as further described below. Suitably, polymerase 3173 has a higher fidelity as compared to commercially available polymerases, e.g., VENT_{R} (New England Biolabs).

Polymerase activity may be determined by one of several methods known in the art. Determination of activity is based on the activity of extending a primer on a template. For example, a labeled synthetic primer may be annealed to a template which extends several nucleotides beyond the 3′ end of the labeled primer. After incubation in the presence of DNA polymerase, deoxynucleotide triphosphates, a divalent cation such as magnesium and a buffer to maintain pH at neutral or slightly alkaline, and necessary salts, the labeled primer may be resolved by, e.g., capillary electrophoresis, and detected. DNA polymerase activity may then be detected as a mobility shift of the labeled primer corresponding to an extension of the primer.

In some embodiments, polymerases of the invention may substantially lack 3′-5′ exonuclease activity. Suitable polymerases substantially lacking 3′-5′ exonuclease activity are shown in SEQ ID NOS: 4, 8, and 14. In some embodiments, the polymerases may be subjected to site-directed mutagenesis, i.e., substitutions, additions or deletions may be introduced, to reduce or eliminate the 3′-5′ exonuclease activity of the native polypeptide. Suitable mutations include those which replace charged amino acids with neutral amino acids in the exonuclease domain of the polymerase. For example, with respect to the polymerase of SEQ ID NO:6, mutations are suitably introduced in the region encompassing amino acid residue 30 to residue 190 of the native polypeptide. Suitably, one or more acidic amino acids (e.g., aspartate or glutamate) in this region are replaced with aliphatic amino acids (e.g., alanine, valine, leucine or isoleucine). Suitably, the aspartate at position 49 and/or the glutamate at position 51 of SEQ ID NO:6 is substituted (see FIG. 8D). Suitably, one or both of these residues are substituted with alanine. The same substitutions at corresponding residues in other polymerases described herein, such residues being depicted as “exonuclease activity” in FIG. 8D (see positions 471 and 473 of alignment depicted in FIG. 8D; see also positions 471 and 473 of alignment depicted in FIG. 7B), also comprise suitable substitutions. As used herein, “corresponding residues” refers to residues from different sequences that do or would align in the same position in a sequence alignment, e.g., Clustal W alignment. Exemplary polymerases subjected to mutagenesis and having substantially reduced 3′-5′ exonuclease activity are shown in SEQ ID NOS:25, 26, and 27.

Determination of whether a polypeptide exhibits exonuclease activity, or in some embodiments, substantially reduced exonuclease activity, may be readily determined by standard methods. For example, polynucleotides can be synthesized such that a detectable proportion of the nucleotides are radioactively labeled. These polynucleotides are incubated in an appropriate buffer in the presence of the polypeptide to be tested. After incubation, the polynucleotide is precipitated and exonuclease activity is detectable as radioactive counts due to free nucleotides in the supernatant.

Some polymerases of the invention may exhibit nick-initiated polymerase activity. As used herein, “nick-initiated polymerase activity” refers to polymerase activity in the absence of exogenous primers which is initiated by single-strand breaks in the template. In these embodiments, synthesis initiates at a single-strand break in the DNA, rather than at the terminus of an exogenous synthetic primer. As will be appreciated, with nick-initiated synthesis, removal of primers is unnecessary, reducing cost, handling time and potential for loss or degradation of the product. In addition, nick-initiated synthesis reduces false amplification signals caused by self-extension of primers. Nick-initiated polymerase activity is particularly suitable for “sequence-independent” synthesis of polynucleotides. As used herein, the term “sequence-independent amplification” is used interchangeably with “whole genome amplification,” and refers to a general amplification of all the polynucleotides in a sample. As is appreciated by those of skill in the art, the term “whole genome amplification” refers to any general amplification method whether or not the amplified DNA in fact represents a “genome,” for example, amplification of a plasmid or other episomal element within a sample. Suitably, nick-initiated polymerase activity can be detected, e.g., on an agarose gel, as an increase in the amount of DNA due to synthesis in the presence of a nicking enzyme as compared to minimal or no product synthesized when nicking enzyme is absent from the reaction.

In some embodiments, the polymerases of the invention may exhibit primer-initiated polymerase activity, and are suitable for sequence-dependent synthesis of polynucleotides. “Sequence-dependent synthesis” or “sequence-dependent amplification” refers to amplification of a target sequence relative to non-target sequences present in a sample. The most commonly used technique for sequence-dependent synthesis of polynucleotides is the polymerase chain reaction (PCR). The sequence that is amplified is defined by the inclusion in the reaction of two synthetic oligonucleotides, or “primers,” to direct synthesis to the polynucleotide sequence intervening between the cognate sequences of the synthetic primers. Thermocycling is utilized to allow exponential amplification of the sequence. As used herein, sequence-dependent amplification is referred to herein as “primer-initiated.” As is appreciated by those of skill in the art, primers may be designed to amplify a particular template sequence, or random primers are suitably used, e.g., to amplify a whole genome. Exemplary polymerases exhibiting primer-initiated polymerase activity have amino acid sequences including but not limited to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:25, SEQ ID NO:26, SEQ ID NO:27, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:61, SEQ ID NO:63, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, or sequence variants thereof.

In some embodiments, the polymerases of the invention may exhibit terminal transferase activity, also referred to in the art as terminal deoxynucleotidyl transferase. As used herein, “terminal transferase activity” refers to the addition of dNTPs to the 3′ terminus of DNA. Enzymes with this activity work on single-stranded DNA (ssDNA), 3′ overhangs of double-stranded DNA (dsDNA), and blunt ends of dsDNA. Such activity does not require a primer, avoiding the need for a separate primer hybridization procedure, and nucleotide additions are not complementary to any template. Because the enzymes with terminal transferase activity can be used with double-stranded DNA, they do not require separate isolation of single-stranded DNA. Exemplary polymerases exhibiting terminal transferase activity have amino acid sequences comprising SEQ ID NO:31 or sequence variants thereof.

In some embodiments, the polymerases of the invention may exhibit primase activity. As used herein, “primase activity” refers to the initiation of genome replication by catalyzing synthesis of an RNA polynucleotide primer on a DNA template in the absence of any other primer. Exemplary polymerases expected to exhibit primase activity have amino acid sequences comprising SEQ ID NO:57 or sequence variants thereof.

In some embodiments, the polypeptides of the invention suitably exhibit reverse transcriptase activity, as exemplified below. “Reverse transcriptase activity” refers to the ability of a polymerase to produce a complementary DNA (cDNA) product from an RNA template. Typically, cDNA is produced from RNA in a modification of PCR, referred to as reverse transcription PCR, or RT-PCR. In contrast to retroviral reverse transcriptases, e.g., those of Moloney Murine Leukemia Virus or Avian Myeloblastosis Virus, the present polymerases may be useful for both reverse transcription and amplification, simplifying the reaction scheme and facilitating quantitative RT-PCR. In contrast to bacterial DNA polymerases, e.g., that of Thermus thermophilus, inclusion of manganese in the RT-PCR reaction buffer is not required using some embodiments of the invention. As is appreciated, manganese may cause a substantial reduction in fidelity. Exemplary polymerases exhibiting reverse transcriptase activity include but are not limited to those having sequences corresponding to SEQ ID NO:6, SEQ ID NO:25, SEQ ID NO:26, SEQ ID NO:27, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77 or sequence variants thereof.

The polypeptides of the invention may exhibit strand displacement activity. As used herein, “strand displacement activity” refers to the ability of a polymerase to displace downstream DNA encountered during synthesis. Protocols such as, e.g., strand displacement amplification (SDA) may exploit this activity. Strand displacement activity may be determined using primer-initiated synthesis. A polymerase of the invention is incubated in the presence of a circular ssDNA template, e.g., M13 phage DNA and its derivatives, and a template-specific primer. A polymerase of the invention may extend the primer the complete circumference of the template at which point the 5′ end of the primer is encountered. If the polymerase is capable of strand displacement activity, the nascent strand of DNA is displaced and the polymerase continues DNA synthesis. The presence of strand displacement activity results in a product having a molecular weight greater than the original template. The higher molecular weight product can be easily detected by agarose gel electrophoresis. Suitable polymerases exhibiting strand displacement activity have amino acid sequences comprising SEQ ID NO:6, SEQ ID NO:25, SEQ ID NO:26, SEQ ID NO:27, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, and sequence variants thereof.

In some embodiments, the purified polymerases may exhibit the enhanced ability to incorporate nucleotide analogs, i.e., polymerases that do not discriminate, or exhibit reduced discrimination, against incorporation of nucleotide analogs. Nucleotide analogs may include chain terminating analogs including acyNTPs, ddNTPs, analogs that have moieties that allow facile detection, including fluorescently labeled nucleotides, e.g., fluorescein or rhodamine derivatives, and/or combinations of chain terminators with detectable moieties, e.g., dye terminators. Nucleotide analogs may also have alternative backbone chemistries, e.g., O-methyl or 2′ azido linkages, alternative ring chemistries, and/or ribonucleotide acids rather than deoxyribonucleotides.

Discrimination of a polymerase against nucleotide analogs can be measured by, e.g., determining kinetics of the incorporation reaction, i.e., the rate of phosphoryl transfer and/or binding affinity for nucleotide analog. Suitably, a polymerase of the invention may have a relative incorporation efficiency of nucleotide analogs that is at least 10% of the incorporation efficiency of deoxynucleotides, i.e., in a reaction including a polymerase of the invention and equimolar amounts of nucleotide analogs and corresponding standard deoxynucleotides, the polymerase is 90% more likely to incorporate the deoxynucleotide. It is appreciated that this embodiment will be particularly suitable for use in sequencing applications, as well as detecting single nucleotide polymorphisms. In other embodiments, the incorporation of nucleotide analogs may aid in the detection of specific sequences by hybridization, e.g., in microarrays, by altering nuclease susceptibility, hybridization strength, selectivity or chemical functionality of a synthetic polynucleotide. Suitably, polymerases of the invention have a relative incorporation efficiency of nucleotide analogs at least about 10% of the incorporation efficiency of standard deoxynucleotides, more suitably at least about 20% incorporation efficiency of standard deoxynucleotides, more suitably at least about 50% incorporation efficiency of standard deoxynucleotides, more suitably at least about 75% incorporation efficiency of standard deoxynucleotides, still more suitably at least about 90% incorporation efficiency of standard deoxynucleotides and most suitably at least about 98-99% incorporation efficiency of standard deoxynucleotides.

Suitable polymerases capable of incorporating nucleotide analogs include sequence variants of the polymerases described herein, wherein the polymerase is mutated in the dNTP binding domain to reduce discrimination against chain terminating analogs. The dNTP binding domain of most polymerases may be characterized as having the sequence KN₁N₂N₃N₄N₅N₆N₇YG/Q, wherein N₁-N₇ are independently any amino acid and N₇ may or may not be present, depending on the polymerase. Most suitably, a substitution is introduced at N₄ of the dNTP binding domain. Most suitably, the amino acid at position N₄ is substituted to tyrosine or a functionally equivalent amino acid that may be chosen by routine experimentation. As an example, a substitution may be made at an amino acid position corresponding to amino acid position 418 of polymerase 3173 or corresponding positions of the other polymerases described herein (see position 843 of alignment depicted in FIG. 8H and position 828 of alignment depicted in FIG. 7D). Suitably, the phenylalanine natively present at position 418 of polymerase 3173 is replaced with tyrosine (“F418Y”). Accordingly, the phenylalanine present at position 9 of Motif B defined by SEQ ID NO:85 is also suitably replaced with a tyrosine. Most suitably, the polymerases exhibit substantially reduced discrimination between chain terminating nucleotides (e.g., nucleotide analogs) and their native counterparts, as shown in the examples. In some cases, a polymerase of the invention discriminates 50 fold less, or 100 fold less, or 500 fold less, or 1000 fold less than its native counterpart.

In other embodiments, the polymerase is a double mutant. Suitably, the native polypeptide of SEQ ID NO:6 may have one mutation in the region encompassing amino acid residue 30 to residue 190 of the native polypeptide sequence and a second mutation at amino acid position 418. Mutations in corresponding residues of the other polymerases described herein, as shown in FIGS. 7A-E and FIGS. 8A-I and described above, are also suitable. Suitably, the double mutant exhibits both reduced exonuclease activity, as described above, and reduced discrimination for incorporation of nucleotide analogs. One example of a double mutant of polymerase 3173 has both a D49A and a F418Y mutation, as shown in SEQ ID NO:27. Another example of a double mutant of polymerase 3173 has both an E51A and a F418Y mutation, as shown in SEQ ID NO:26.

The invention further provides compositions including polymerases of the invention. In some embodiments, compositions of the invention include one or more polymerases selected from SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:25, SEQ ID NO:26, SEQ ID NO:27, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:61, SEQ ID NO:63, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, or sequence variants thereof. In a particular embodiment, the composition comprises SEQ ID NO:6 and one or more polymerases selected from SEQ ID NO:25, SEQ ID NO:26, SEQ ID NO:27 and sequence variants thereof. In other embodiments, polymerases of the invention can be included in a composition with other commercially available polymerases.

Some embodiments of the invention provide reagents for expressing the polymerases described herein. Such reagents can be used for the production of the polymerases.

Some versions of the reagents for expressing the polymerases include isolated polynucleotides encoding the polymerases. The term “isolated polynucleotide” is inclusive of, for example: (a) a polynucleotide which includes a coding sequence of a portion of a naturally occurring genomic DNA molecule that is not flanked by coding sequences that flank that portion of the DNA in the genome of the organism in which it naturally occurs; (b) a polynucleotide incorporated into a vector or into the genomic DNA of a prokaryote or eukaryote in a manner such that the resulting molecule is not identical to any naturally occurring vector or genomic DNA; and (c) a cDNA molecule, a genomic fragment, a fragment produced by polymerase chain reaction, or a restriction fragment. A “vector” is any polynucleotide entity capable of being replicated by standard cloning techniques.

Suitable polynucleotides encoding a polymerase of the invention have the nucleotide sequence shown in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:60, SEQ ID NO:62, SEQ ID NO:64, SEQ ID NO:65, SEQ ID NO:66, and sequence variants thereof.

Some reagents for expressing the polymerases include DNA constructs useful in preparing the polypeptides of the invention. The DNA constructs include at least one polynucleotide encoding a polypeptide described herein operably connected to a promoter. The promoter may be natively associated with the coding sequence or may be heterologous. “Heterologous” refers to sequence portions not natively associated with a sequence. Suitable promoters are constitutive and inducible promoters. A “constitutive” promoter is a promoter that is active under most environmental and developmental conditions. Examples of constitutive promoters include but are not limited to T7 promoters, cytomegalovirus promoters such as the CMV immediate early promoter, SV40 early promoter, mouse mammary tumor virus promoter, human immunodeficiency virus promoters such as the HIV long terminal repeat promoter, maloney virus promoter, Epstein Barr virus promoter, rous sarcoma virus promoter, ALV, B-cell specific promoters, and baculovirus promoter for expression in insect cells. An “inducible” promoter is a promoter that is under environmental or developmental regulation. Examples of inducible promoters include the lac promoter, such as the lacUV5 promoter or the T7-lac promoter, copper-inducible promoters (Gebhart et al. Eukaryotic Cell 2006 5(6):935-44), and “tet-on” and “tet-off” promoters.

The term “operably connected” refers to a functional linkage between a promoter and a second nucleic acid sequence, wherein the promoter directs transcription of the nucleic acid corresponding to the second sequence. The constructs may suitably be introduced into host cells, such as E. coli or other suitable hosts known in the art for producing polymerases of the invention.

Some reagents for expressing the polymerases include hosts capable of expressing the polymerases described herein. Suitable hosts include both eukaryotic and prokaryotic hosts, such as mammalian-, bacterial-, fungal-, and insect-derived hosts. Examples of bacterial hosts include Escherichia, Salmonella, Bacillus, Clostridium, Streptomyces, Staphyloccus, Neisseria, Lactobacillus, Shigella, and Mycoplasma. E. coli strains, such as BL21(DE3), C600, DH5αF′, HB101, JM83, JM101, JM103, JM105, JM107, JM109, JM110, MC1061, MC4100, MM294, NM522, NM554, TGI, χ¹⁷⁷⁶, XL1-Blue, and Y1089⁺, all of which are commercially available. Other expression hosts are well known in the art.

The present invention further provides a method of synthesizing a copy or complement of a polynucleotide template. The method includes a step of contacting the template with a polypeptide of the invention under conditions sufficient to promote synthesis of the copy or complement. In some embodiments, the template is RNA. In other embodiments, the template is DNA. In yet other embodiments, both RNA and DNA templates are used.

One example of a method in which both RNA and DNA templates are used includes “single-tube” RT-PCR. In such a method, both reverse transcription of RNA to DNA and amplification of the DNA occur within a single tube with a single enzyme carrying out the reverse transcription and PCR amplification steps. Single-tube RT-PCR preferably allows for the reverse transcription and PCR steps to occur sequentially without the addition of an additional enzyme or reagent(s) between the steps. In general, such a method includes synthesizing a copy or complement of a polynucleotide template comprising contacting the template with a polymerase under conditions sufficient to promote synthesis of the copy or complement, wherein: the polynucleotide template comprises an RNA template and a DNA template; the copy or complement comprises a first DNA copy or complement and a second DNA copy or complement, wherein the first DNA copy or complement is the DNA template; the polymerase synthesizes the first DNA copy or complement from the RNA template; and the polymerase synthesizes the second DNA copy from the DNA template. Examples of polymerases having both RNA-dependent (i.e., reverse transcriptase) and DNA-dependent polymerase activity for use in single-tube RT-PCR include those with sequences corresponding to SEQ ID NO:6, SEQ ID NO:25, SEQ ID NO:26, SEQ ID NO:27, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77 or sequence variants thereof.

A copy or complement of a polynucleotide template may be synthesized using a polymerase of the invention in a thermocycled reaction, e.g., PCR, RT-PCR, or alternatively, using substantially isothermal conditions. As used herein, “substantially isothermal” refers to conditions that do not include thermocycling. Due to their thermostability, the present polypeptides may prove particularly useful in, e.g., strand-displacement amplification (SDA), loop-mediated isothermal amplification (LAMP), rolling circle amplification (RCA) and/or multiple displacement amplification (MDA). Using these techniques, nucleic acids from clinical isolates containing human cells can be amplified for genotyping. Nucleic acids from clinical isolates containing viruses or bacterial cells can be amplified for pathogen detection. Nucleic acids from microbial cells, which may be very difficult to isolate in large quantities, may be amplified for gene mining or enzyme or therapeutic protein discovery.

In some methods of the invention, amplification is carried out in the presence of at least one primer pair, e.g., to amplify a defined target sequence. In other embodiments, random primers are added to promote sequence-independent amplification. In still further embodiments, primers are excluded, and a nick-inducing agent is optionally added to facilitate polymerase activity. A “nick-inducing agent” is defined herein as any enzymatic or chemical reagent or physical treatment that introduces breaks in the phosphodiester bond between two adjacent nucleotides in one strand of a double-stranded nucleic acid. The nicks may be introduced at defined locations, suitably by using enzymes that nick at a recognition sequence, or may be introduced randomly in a target polynucleotide. Examples of nick-inducing enzymes include Nb.Bpu10I (Fermentas Life Sciences), Nt.BstNB I, Nt.Alw I, Nb.BbvC I, Nt.BbvC I, Nb.Bsm I, Nb.BsrD (New England Biolabs) and E. coli endonuclease I.

Due to their unique biochemical properties, the polymerases of the present invention may be particularly suitable for amplifying sequences that are traditionally difficult to amplify. These sequences are referred to herein as “amplification-resistant sequences.” For example, some difficult sequences have inverted repeats in their sequences that promote the formation of DNA secondary structure. Others have direct repeats that cause the nascent strand to spuriously re-anneal and cause incorrect insertion or deletion of nucleotides. In other cases, amplification-resistant sequences have a high content of guanine and cytosine (G+C) or, conversely, a high content of adenine and thymidine (A+T) residues. A sequence has a high content of G+C or A+T when at least about 65% of the sequence comprises those residues. In some embodiments, a sequence is considered amplification-resistant when the desired product is at least about 2 kb. In some cases, polymerases of the invention can amplify sequences that are larger than the normal range of PCR, i.e., around 10 kb, as exemplified below. In other cases, polymerases of the invention can amplify sequences that are prone to mispriming, as exemplified below.

The polymerases of the invention may be characterized by their thermostability, temperature optimum, fidelity of incorporation of nucleotides, cofactor requirements, template requirements, reaction rate, affinity for template, affinity for natural nucleotides, affinity for synthetic nucleotide analogs and/or activity in various pHs, salt concentrations and other buffer components. As will be appreciated by the skilled artisan, an appropriate polymerase, or combination of polymerases, may be selected based on any of these characteristics or combinations thereof, depending on the application of interest.

The following examples are provided to assist in a further understanding of the invention. The particular materials and conditions employed are intended to be further illustrative of the invention and are not limiting upon the reasonable scope of the appended claims.

### EXAMPLES

### Example 1

### Isolation of Uncultured Viral Particles from a Thermal Spring

Viral particles were isolated from a thermal spring in the White Creek Group of the Lower Geyser Basin of Yellowstone National Park (N 44.53416, W 110.79812; temperature 80° C., pH 8), commonly known as Octopus Spring. Thermal water was filtered using a 100 kiloDalton molecular weight cut-off (mwco) tangential flow filter (A/G Technology, Amersham Biosciences) at the rate of 7 liters per minute for over 90 minutes (630 liters overall), and viruses and microbes were concentrated to 2 liters. The resulting concentrate was filtered through a 0.2 μm tangential flow filter to remove microbial cells. The viral fraction was further concentrated to 100 ml using a 100 kD tangential flow filter. Of the 100 ml viral concentrate, 40 ml was processed further. Viruses were further concentrated to 400 μl and transferred to SM buffer (0.1 M NaCl, 8 mM MgSO4, 50 mM Tris HCl 7.5) by filtration in a 30 kD mwco spin filter (Centricon, Millipore).

### Example 2

### Isolation of Viral DNA

Serratia marcescens endonuclease (Sigma, 10 U) was added to the viral preparation described in Example 1 to remove non-encapsidated (non-viral) DNA. The reaction was incubated for 30 min. at 23° C. Subsequently, EDTA (20 mM) and sodium dodecyl sulfate (SDS) (0.5%) was added. To isolate viral DNA, Proteinase K (100 U) was added and the reaction was incubated for 3 hours at 56° C. Sodium chloride (0.7M) and cetyltrimethylammonium bromide (CTAB) (1%) were added. The DNA was extracted once with chloroform, once with phenol, once with a phenol:chloroform (1:1) mixture and again with chloroform. The DNA was precipitated with 1 ml of ethanol and washed with 70% ethanol. The yield of DNA was 20 nanograms.

### Example 3

### Construction of a Viral DNA Library

Ten nanograms of viral DNA isolated as described in Example 2 was physically sheared to between 2 and 4 kilobases (kb) using a HydroShear Device (Gene Machines). These fragments were ligated to double-stranded linkers having the nucleotide sequences shown in SEQ ID NOS:21 and 22 using standard methods. The ligation mix was separated by agarose gel electrophoresis and fragments in the size range of 2-4 kb were isolated. These fragments were amplified by standard PCR methods. The amplification products were inserted into the cloning site of perSMART vector (Lucigen, Middleton, Wis.) and used to transform E. CLONI 10 G cells (Lucigen, Middleton, Wis.).

### Example 4

### Screening by Sequence Similarity

21,797 clones from the library described in Example 3 were sequenced using standard methods. These sequences were conceptually translated and compared to the database of non-redundant protein sequences in GenBank (NCBI) using the BLASTx program (NCBI). Of these, 9,092 had significant similarity to coding sequences of known proteins in the database. 2,036 had similarity to known viral coding sequences. 148 had at least partial similarity to known DNA polymerase coding sequences. 34 appear to be complete polymerase coding sequences.

### Example 5

### Expression of DNA Polymerase Genes

34 complete polymerase genes from the library described in Examples 3 and 4, as well as 24 additional viral genes from three other similarly prepared libraries, were constitutively expressed in the E. CLONI 10 G cells (Lucigen, Middleton, Wis.). The proteins were extracted, heated to 70° C. for 10 minutes and tested for DNA polymerase activity using a primer extension assay as follows.

A primer of 37 nucleotides having the sequence shown in SEQ ID NO:23, labeled on its 5′ end with ROX, was annealed to a template of 41 nucleotides having the sequence shown in SEQ ID NO:24. Proteins extracted as described above and template were added to 20 mM Tris-HCl, 10 mM (NH₄)₂SO₄, 10 mM KCl, 2 mM MgSO₄, 0.1% Triton X-100, pH 8.8 at 25° C., and 250 μM each of deoxycytidine triphosphate (dCTP), deoxyadenine triphosphate (dATP), deoxyguanidine triphosphate (dGTP), and thymidine triphosphate (TTP). The reaction was incubated at 70° C. for 10 minutes. The reactions were analyzed using an ABI 310 Genetic Analyzer. Extension of the primer resulted in a mobility shift corresponding to an extension of 4 nucleotides that was detectable by the ABI 310 Genetic Analyzer. Of the 58 clones tested, a total of ten clones expressed detectable DNA polymerase (DNAP) activity. The clone number and corresponding polynucleotide sequence, polypeptide sequence, sequence similarity and E (expect)-values for these polymerases are shown below in Table 1. The presence of 3′-5′ exonuclease activity resulted in a reaction product migrating at less than 37 nucleotides during capillary electrophoresis.

TABLE 1

| | | | | Expect | % | % | |
|---|---|---|---|---|---|---|---|
| Clone | Polynuclcotide | Polypeptide | Strongest similarity | value | identity | conserved | Exo |
| 3063 | SEQ ID NO. 1 | SEQ ID NO. 2 | Aquifex pyrophilus pol I | 0.0 | 63 | 79 | 3′ |
| 488 | SEQ ID NO. 3 | SEQ ID NO. 4 | Aquifex pyrophilus pol I | 1 × 10⁻⁴⁶ | 33 | 51 | No |
| 3173 | SEQ ID NO. 5 | SEQ ID NO. 6 | Desulfitobacterium | 2 × 10⁻³⁷ | 30 | 48 | 3′ |
| | | | hafniense pol I | | | | |
| 4110 | SEQ ID NO. 7 | SEQ ID NO. 8 | Pyrodictium occultum | 3 × 10⁻⁵⁵ | 28 | 46 | No |
| | | | pol II | | | | |
| 2323 | SEQ ID NO. 9 | SEQ ID NO. 10 | Pyrobaculum aerophilum | 1 × 10⁻⁴⁷ | 28 | 45 | 3′ |
| | | | pol II | | | | |
| 653 | SEQ ID NO. 11 | SEQ ID NO. 12 | Pyrococcus furiosus | 2 × 10⁻¹² | 37 | 59 | 3′ |
| | | | virus pol | | | | |
| 967 | SEQ ID NO. 13 | SEQ ID NO. 14 | Aquifex aeolicus pol I | 3 × 10⁻⁴⁴ | 36 | 53 | No |
| 2783 | SEQ ID NO. 15 | SEQ ID NO. 16 | Sulfolobus tokodaii pol II | 3 × 10⁻⁵⁶ | 27 | 46 | 3′ |
| 2072 | SEQ ID NO. 17 | SEQ ID NO. 18 | Sulfolobus tokodaii pol II | 2 × 10⁻¹⁰ | 39 | 60 | ND |
| 2123 | SEQ ID NO. 19 | SEQ ID NO. 20 | Pyrococcus abyssi pol II | 1 × 10⁻⁴ | 35 | 51 | ND |

### Example 6

### Purification and Characterization of Viral DNA Polymerase Identified in the Viral Libraries

As determined by sequence similarity screening described in Example 4, the polynucleotide having the sequence of nucleotides shown in SEQ ID NO:5 included regions having significant similarity to several dozen sequences encoding bacterial DNA polymerase I. The E value for the complete gene was as low as 2×10⁻³⁷, indicating a very high probability that the sequence is that of an authentic DNA polymerase gene. This coding sequence was transferred to a tac-promoter based expression vector (Lucigen) and used to produce high levels of thermostable DNA polymerase in E. CLONI 10 G cells according to the manufacturer's recommendations (Lucigen). The protein was purified by column chromatography.

To measure the activity of the polymerase, the purified protein was incubated with 50 μl of mix containing 0.25 mg/ml activated calf thymus DNA (Sigma), 200 μM each of deoxycytidine triphosphate (dCTP), deoxyadenine triphosphate (dATP), deoxyguanidine triphosphate (dGTP), and thymidine triphosphate (TTP), 100 μCi/ml of [α P-33] deoxycytidine triphosphate (Perkin-Elmer), 20 mM Tris-HCl, 10 mM (NH₄)₂SO₄, 10 mM KCl, 2 mM MgSO₄, 0.1% Triton X-100, pH 8.8 at 25° C. The reaction was incubated at 60° C. for 30 minutes. The reaction product (5 μl) was transferred to a DE81 filter (Whatman) and allowed to dry. The filter was washed with 3 changes of 5M sodium phosphate (pH 7.0), water and with ethanol. The filter was dried and incorporated label was measured in a scintillation counter. A blank reaction without added DNA polymerase was used to determine background activity. Activity of the polymerase was determined by the following equation, widely used in the art and reported in standard units:

Activity=(sample counts−blank)×(8 nmol dNTPs/reaction)×(1 unit/10 nmol dNTPs incorporated)

Counts of >1,000 cpm were detected compared to a typical background of <100 cpm, confirming the presence of DNA polymerase activity.

### Example 7

### Production of Exonuclease Deficient Polymerase 3173 Mutants

The presence of a 3′-5′ exonuclease domain in the 3173 DNA polymerase was detected by reduction in molecular weight of a 5′ fluorescently labeled oligonucleotide. Upon incubation of the primer/template complex described in Example 5, under the same conditions, a portion of the primer product was reduced in apparent molecular weight. This reduction in size was detected by capillary electrophoresis using an ABI 310 Genetic Analyzer operated in GeneScan mode. The presence of an exonuclease domain was confirmed by sequence alignment and by incubation of the polymerase with a radiolabeled polynucleotide, followed by digestion and precipitation with trichloroacetic acid. Radioactivity due to free nucleotides in the supernatant was measured.

Based on sequence alignments comparing polymerase 3173 with sequences identified in NCBI conserved domain database cdd.v2.07 (publicly available), an active site and apparent metal chelating amino acids (amino acids D49 and E51) were identified. Based on this information, two mutants of polymerase 3173 were produced. One mutant, D49A, was the result of a mutation of the aspartic acid at position 49 of the wild-type protein to alanine. The second mutant, E51A, was the result of a mutation of the glutamic acid at position 51 of the native protein to alanine. Mutants D49A and E51A were produced using standard methods.

An exonuclease assay was performed to confirm that exonuclease activity was eliminated in the mutants. Each of mutants D49A and E51A were tested for exonuclease activity using the radioactive nucleotide release assay described above, which is capable of detecting exonuclease activity levels below 0.1% of wild-type. Wild-type polymerase 3173 exhibited potent nuclease activity, whereas neither mutant exhibited detectable nuclease activity.

### Example 8

### Processivity of Polymerase 3173 Mutant D49A

Processivity was determined by annealing a fluorescently-labeled primer to a single-stranded M13 template (50 nM each). Polymerase 3173 mutant D49A was added (0.5 nM) and allowed to associate with the primed template. Nucleotides were added simultaneously with an “enzyme trap” comprised of an excess of activated calf thymus DNA (Sigma) (0.6 mg/ml final) and the reactions were incubated at 70° C. Samples were removed and the reactions were quenched by EDTA (10 mM) at 1, 3, 10, and 30 minutes. Extension of the primer before dissociation was measured by resolving the extension product on an ABI 310 Genetic Analyzer in GeneScan mode. Removal of product at the increasing time points resulted in increasingly high molecular weight product until a maximum was reached. The shortest time point giving maximal product size was used for the calculations. Peaks from the electropherograms were integrated by the GeneScan software and processivity was determined by the following equation:

Processivity=[[(1×I(1))]+[(2×I(2))]+ . . . [(n)×(I(n))]]/[I(1)+I(2) . . . +I(n)]]

where I=intensity of each peak, n=number of nt added. The processivity for polymerase 3173 D49A was determined to be 47 nt.

### Example 9

### Characterization of Polymerase 3173

Exonuclease activity for polymerase 3173 was determined as described in Example 7.

The binding constant (reported as Km, the concentration at which the reaction rate is 50% maximal) for nucleotides by polymerase 3173 was determined using activated calf thymus DNA as a template. Reactions were maintained under pseudo-first order conditions using a molar excess of all components, with the exceptions of the enzyme and the nucleotides. Reactions (50 μl) were incubated at 70° C. and samples (5 μl each) were removed at varying time points and spotted on DE81 paper. Activity was determined as described in Example 6. The binding constant for primed template was similarly determined except that nucleotides were supplied in excess and the concentration of primed template (primed single stranded M13 DNA) was varied. Results are shown in Table 2 below.

TABLE 2
Polymerase 3173 Activity Characteristics

| Activity | 3173 |
|---|---|
| 5′-3′ exonuclease activity | — |
| 3′-5′ exonuclease activity | Strong |
| Strand displacement | Strong |
| Extension from nicks | Strong |
| Thermostability (T_{1/2} at 95°) | 10 min. |
| Km dNTPs | 20-40 μM |
| Km DNA | 5.3 nM |
| Fidelity | 6.98 × 10⁴ |

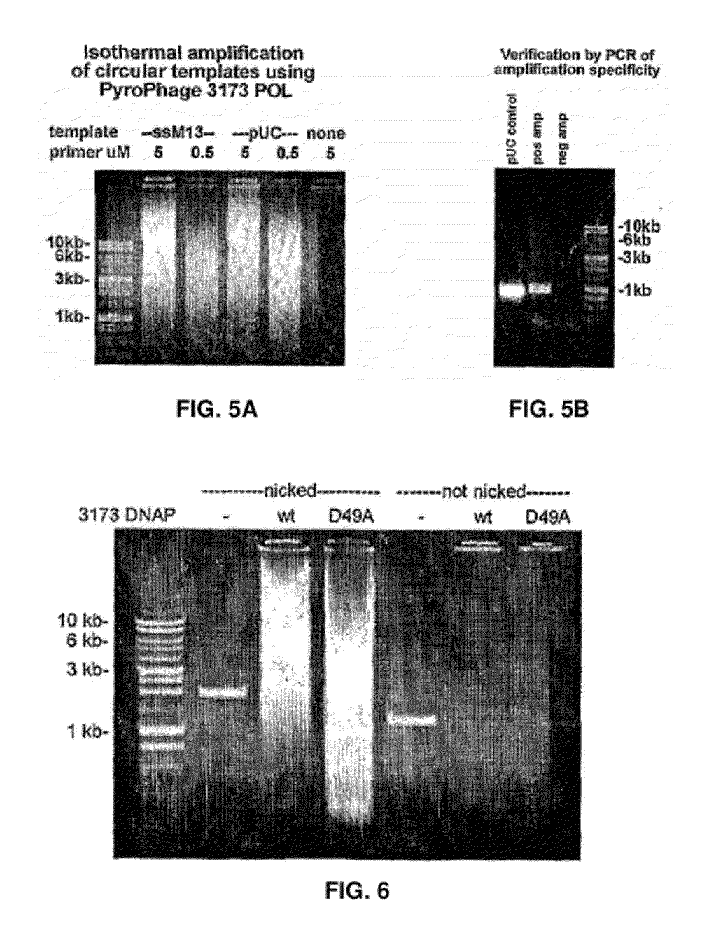

Strand displacement activity was determined using primer-initiated synthesis in a rolling circle amplification (RCA) protocol. Briefly, polymerase 3173 was incubated in the presence of a plasmid and random primers. Polymerase 3173 extended the primer the complete circumference of the plasmid at which point the 5′ end of the primer was encountered. Polymerase 3173 displaced the nascent strand of DNA and continued DNA synthesis. The presence of strand displacement activity resulted in a product having a molecular weight greater than the original template. As shown in FIGS. 5A, 5B, and 6, the higher molecular weight product was easily detected by agarose gel electrophoresis.

Fidelity was determined as described in example 10.

### Example 10

### High Fidelity PCR Using Polymerase 3173

Fidelity was determined by a modification of the standard assay in which the lacIq gene is amplified by the DNA polymerase of interest and inserted into a plasmid containing genes encoding a functional lacZ alpha peptide and a selectable marker. Primers of SEQ ID NOS:28 and 29 were used to amplify a sequence containing both the lacIq and the KanR gene. Insertion of this gene into the Eco109I site of pUC19 resulted in double resistance to kanamycin and ampicillin. Normally a white phenotype is seen for a clone containing this construct when plated on X-Gal. Mutation of the lacIq results in a blue phenotype for the colonies when plated on X-Gal. The wild-type (proofreading) DNA polymerase 3173 and its exonuclease deficient derivatives, E51A and D49A, and, for comparison, two standard DNA polymerases, Taq and VENT_{R} DNA polymerases, were tested.

For high fidelity PCR amplification, five units of the wild-type (proofreading) DNA polymerase 3173 (SEQ ID NO:6) was tested using the following mix (50 mM Tris HCl (pH 9.0 at 25° C.), 50 mM KCl, 10 mM (NH₄)₂SO₄, 1.5 mM MgSO₄, 1.5 mM MgCl₂, 0.1% triton-X100, 250 mM ectoine and 0.2 mM each of dGTP, dATP, dTTP and dCTP. Opposing primers of SEQ ID 28 and 29 (1 μM each) amplified the expected 2 k kb product from template SEQ ID 30 (10 ng). After thermal cycling (94° C. for 1 minute, 25 cycles of (94° C. for 15 seconds, 60° C. for 15 seconds, 72° C. for 2.5 minutes) and 72° C. 7 minutes), reaction products were quantified to determine “fold amplification,” (see below) using agarose gel electrophoresis. Both primers contain Eco109I sites. The PCR product was digested with Eco109I and inserted into the Eco1091 site of pUC19. 10 G cells transformed by the construct were plated on LB plates containing ampicillin (100 μg/ml), kanamycin (30 μg/ml) and X-Gal (50 μg/ml). Blue and white colony counts were used for the fidelity determinations. For comparison, polymerase 3173 exonuclease deficient mutants, E51A and D49A and, two standard DNA polymerases, Taq and VENT_{R} DNA polymerases, were tested in the same manner.

As is standard in the art, fidelity was determined based on the ratio of blue:white colonies using the following equation:

fidelity=ln F/d×t

where F=fraction of white colonies, d=number of duplications during PCR (log 2 of fold amplification) and t is the effective target size (349 for lacIq). The results of the fidelity assay are shown in Table 3 below.

TABLE 3
Fidelity of Polymerases

| DNA polymerase | fidelity |
|---|---|
| DNA polymerase 3173 | 6.98E+04 |
| DNA polymerase 3173 (E51A) | 1.28E+04 |
| DNA polymerase 3173 (D49A) | 1.88E+04 |
| Taq | 9.76E+03 |
| VENT_{R} | 2.42E+04 |

### Example 11

### Polymerase Chain Reaction Using Polymerase 3173 Mutant D49A

Primers specific for the bla gene of pUC19 were used to amplify a 1 kb product using polymerase 3173 mutant D49A and commercial enzymes for comparison. The polymerase chain reactions included 50 mM Tris HCl (pH 9.0 at 25° C.), 50 mM KCl, 10 mM (NH₄)₂SO₄, 1.5 mM MgSO₄, 1.5 mM MgCl₂, 0.1% triton-X100, 0.02 mg/ml bovine serum albumin, 250 mM ectoine and 0.2 mM each of dGTP, dATP, dTTP and dCTP. Opposing primers annealing 1 kb apart in the bla gene of the pUC19 plasmid and the D49A mutant polymerase were added. After thermal cycling (25 cycles of 94° C. for 15 seconds, 60° C. for 15 seconds, 72° C. for 60 seconds), reactions were resolved using agarose gel electrophoresis.

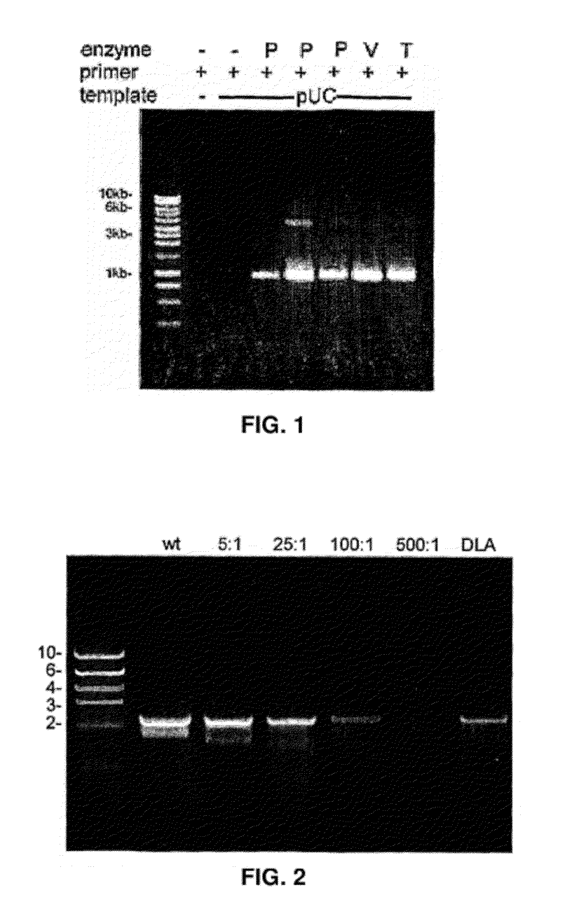

The results are shown in FIG. 1. Lanes are as follows: no template DNA (lane 2) or 40 nanograms of pUC19 DNA (lanes 3-8); no enzyme (lanes 2 and 3), 2, 4 or 8 Units of polymerase 3173 mutant D49A (P, lanes 4, 5 and 6, respectively), 5 U VENT_{R} (V, NEB, lane 7) or 5 U Taq DNA polymerase (T, Lucigen, lane 8). Also shown are molecular weight markers (lane 1).

As seen in FIG. 1, PCR amplification using the D49A mutant resulted in a product of the predicted size, similar to commercially available enzymes.

### Example 12

### Polymerase Chain Reaction Using Polymerase 3173 and Polymerase 3173 Mutant E51A

A range of mixes of polymerase 3173 and polymerase 3173 mutant E51A (1:5, 1:25, 1:100, 1:500 U/U), and primers of SEQ ID NO:58 and SEQ ID NO:59, were used to amplify a 2259 nucleotide region of a circular synthetic template. The amplification mix, comprised of 50 mM Tris HCl (pH 9.0 at 25° C.), 50 mM KCl, 10 mM (NH₄)₂SO₄, 1.5 mM MgSO₄, 1.5 mM MgCl₂, 0.1% triton-X100, 15% sucrose, 0.2 mM each of dGTP, dATP, dTTP and dCTP, 1 μM of each opposing primer and 20 ng of template, was incubated under the following conditions: 94° C. for 2 minutes, 25 cycles of (94° C. for 15 seconds, 69° C. for 15 seconds, 72° C. for 2 minutes) and 72° C. for 10 minutes. The amplification reaction resulted in product migrating at the expected molecular weight with no extraneous products as seen in FIG. 2.

### Example 13

### PCR Amplification of the cyc Gene from Bacillus stearothermophilus

The cyc gene from a Bacillus stearothermophilus isolate had proven to be an amplification-resistant sequence by all commercially available DNA polymerases that were tested. This sequence was amplified using polymerase 3173 mutant D49A using the conditions described in Example 10. For comparison, amplification of this gene by other commercially available DNA polymerases including Taq, Phusion (Finnzymes), VENT_{R}, Tfl (Promega), KOD (TaKaRa) was also conducted according to each manufacturers' recommendations.

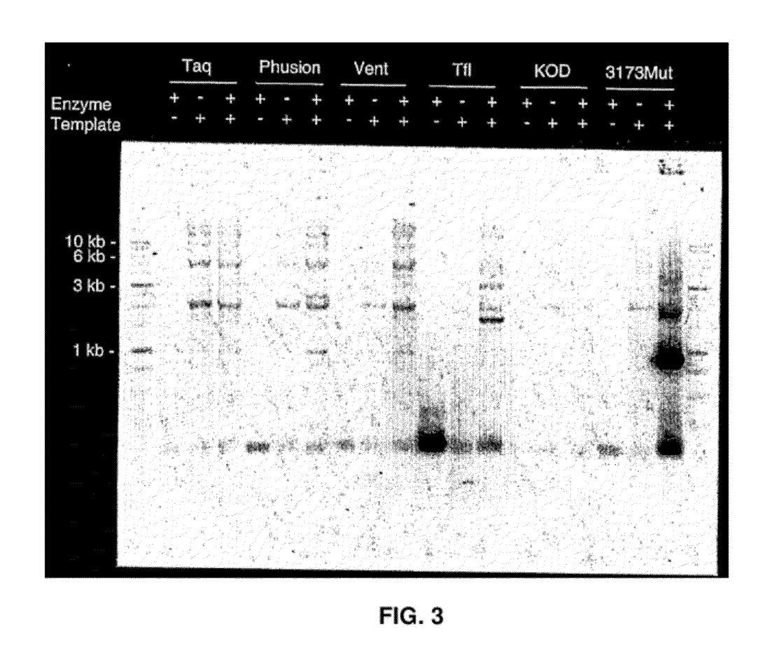

The results are shown in FIG. 3. Lanes are as follows: Taq (lanes 2-4), Phusion (lanes 5-7), VENT_{R} (lanes 8-10), Tfl (lanes 11-13), KOD (lanes 14-16) and polymerase 3173 mutant D49A (lanes 17-19). Amplification products were resolved by agarose gel electrophoresis and imaged using standard methods. The predicted amplification product comigrates with the 1 kb marker (lanes 1 and 20). Negative control reaction lacking template (lanes 2, 5, 8, 11, 14 and 17) or enzyme (lanes 3, 6, 9, 12, 15 and 18) are also shown in FIG. 3.

As shown in FIG. 3, amplification was observed using commercially available enzymes, as well as the D49A mutant, however, none of these commercially available enzymes resulted in the exceptionally high yields generated using mutant D49A.

### Example 14

### Reverse Transcriptase Activity and RT-PCR Using Polymerase 3173 and Polymerase 3173 Mutants

Reverse transcriptase activity was detected by incorporation of radiolabeled deoxyribonucleotide triphosphates into polydeoxyribonucleotides using a ribonucleic acid template. A reaction mix comprising 50 mM Tris-HCl pH 8.3 at 25° C., 75 mM KCl, 3 mM MgCl₂, 2 mM MnCl₂, 200 μM dTTP, 0.02 mg/ml Poly rA: Oligo dT (Amersham), and 10 μCi of [P-32] alpha dTTP was incubated with 1 U of polymerase 3173 or the polymerase 3173 mutant D49A at 60° C. for 20 minutes. Incorporation of dTTP was detected as radioactive counts adhering to DE81 filter paper. Similar reverse transcription reactions were measured by incorporation of labeled dTTP on a poly rA template using 1 unit of Tth (Promega) and 1 unit MMLV reverse transcriptase (Novagen) according to the respective manufacturers' recommended conditions. Incorporation rates of polymerase 3173 and mutant D49A in comparison to commercially available enzymes are shown in Table 4 below.

TABLE 4
Reverse Transcriptase Activity of Polymerases

| Enzyme | Incorporation of dTTP |
|---|---|
| 3173 wt | 1.037 nmoles |
| 3173 (D49A) | 1.507 nmoles |
| Tth DNA polymerase | 0.802 nmoles |
| MMLV reverse transcriptase | 1.110 nmoles |

In addition, in contrast to the manganese-dependent activity of Tth, reverse transcription by polymerase 3173 and mutant D49A is equivalent when reactions are run in the presence of either manganese or magnesium.

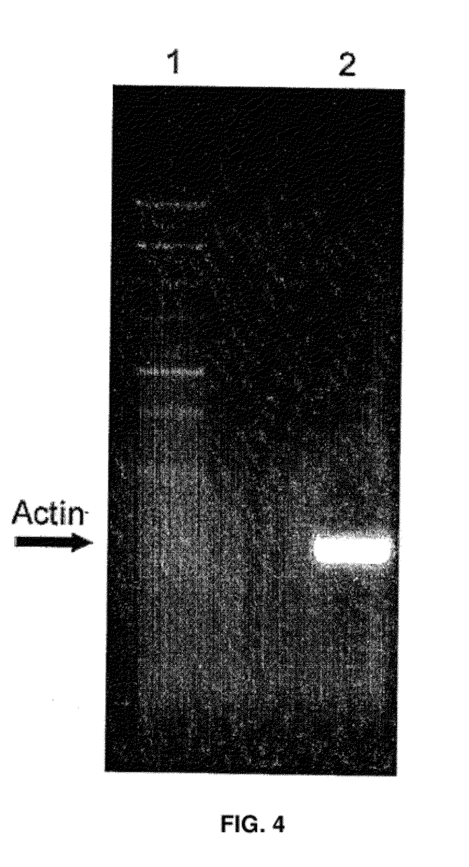

Next, a 50 μl reaction containing 20 mM Tris-HCl (pH 8.8 at 25° C.), 10 mM (NH4)2SO4, 10 mM KCl, 2 mM MgSO4, 0.1% Triton X-100, 0.25M ectoine, 200 μM each of dGTP, dATP, dTTP and dCTP, 1 μg of total mouse liver RNA (Ambion), 0.4 μM of primers from the QuantumRNA β-actin Internal Standards kit (Ambion) and 5 units of polymerase 3173 mutant E51A DNA polymerase was incubated under the following temperature cycle: 60° for 60 minutes, 94° C. for 2 minutes, 35 cycles of (94° C. for 15 seconds, 57° C. for 15 seconds, 72° C. for 1 minute), followed by 72° C. for 10 minutes. The primers are predicted to direct synthesis of a 294 base-pair product. Five μl of the reaction was analyzed by agarose gel electrophoresis. As shown in FIG. 4, a prominent band was observed migrating at the predicted molecular weight; no other bands were observed.

### Example 15

### High Temperature Isothermal RCA Amplification

Five units of polymerase 3173 was used to amplify one nanogram each of single-stranded M13mp18 and double stranded pUC19 plasmid DNA. Reactions contained 20 mM Tris-HCl, 10 mM (NH₄)₂SO₄, 10 mM KCl, 2 mM MgSO₄, 0.1% Triton X-100, pH 8.8 at 25° C., and 250 μM each of dGTP, dATP, dTTP and dCTP. Either 0.5 μM or 5 μM of random decamer primers were added to each template. Reactions were incubated at 95° C. prior to addition of enzyme, then 16 hours at 55° C. with enzyme. One fiftieth of each reaction was resolved on a 1% agarose gel.

Results are shown in FIG. 5A. Lanes are as follows: five units of 3173 wild type DNA polymerase used to amplify M13mp18 single-stranded DNA template (lanes 2 and 3) and pUC19 double-stranded DNA (lanes 4 and 5) or no template (lane 6). Random ten nucleotide oligomer primers are added in the concentrations of 5 μM (lanes 2, 4 and 6) or 0.5 μM (lanes 3 and 5).

As shown in FIG. 5A, polymerase 3173 amplified both single- and double-stranded DNA templates. The estimated overall yield was approximately 50 μg for both templates, indicating amplification of up to 50,000-fold. A negative control reaction lacking template resulted in no significant yield of amplification product.

To determine if the amplification was specific for the template DNA, one μl of the amplification product of the positive pUC19 reaction was tested in a PCR reaction using primers specific for a 1 kb sequence in the bla gene of the original plasmid template. As a negative control, a reaction lacking deoxynucleotides was analyzed using PCR. As a positive control, the 1 kb sequence was amplified directly from 1 ng of pUC19.

Results are shown in FIG. 5B. Lane 1 shows positive control amplification of the 1 kb bla gene sequence of pUC19. Lane 2 shows amplification of the bla gene from the product amplified as described above. Lane 3 shows the results for the negative control.

As expected, authentic amplification product was obtained using polymerase 3173. The 1 kb amplification product was detected by PCR in the test amplification reaction and in the positive control reaction, but not in the negative control amplification reaction.

### Example 16

### Isothermal RCA in the Absence of Added Primers

Reactions containing 10 ng of plasmid DNA, 20 mM Tris-HCl, 10 mM (NH₄)₂SO₄, 10 mM KCl, 2 mM MgSO₄, 0.1% Triton X-100, pH 8.8 at 25° C., and 200 μM each of dGTP, dATP, dTTP and dCTP were incubated for 2 hours at 56° C. with or without 10 units of nick-generating enzyme N.Bst NB1 (NEB) and either no DNA polymerase, 200 units of 3173 wt or 400 units of 3173 (D49A) mutant enzyme. Parallel reactions were performed in the absence of nicking enzyme, polymerase or both. Amplification products were analyzed by agarose gel electrophoresis.

Results are shown in FIG. 6. Lanes are as follows: Nicking enzyme present (lanes 2-4) or absent (lanes 5-7). Polymerase 3173 (lanes 3 and 6) or D49A mutant (lanes 4 and 7). As shown in FIG. 6, multi-microgram yields of DNA product were obtained in the presence of both polymerase 3173 and the polymerase 3173 mutant D49A when the nicking enzyme was present, but not the absence of DNA polymerase or nicking enzyme.

### Example 17

### Mutagenesis of the Polymerase Domain to Reduce Nucleotide Discrimination

A 5′ Rox-labeled primer complementary to M13mp18 nucleotides 6532 to 6571 (5 nM) was annealed to single-stranded M13mp18 DNA (10 nM) in a buffer containing 20 mM Tris-HCl, 10 mM (NH₄)₂SO₄, 10 mM KCl, 2 mM MgSO₄, 0.1% Triton X-100, pH 8.8 at 25° C., and 50 μM each of dGTP, dATP, dTTP and dCTP. In separate reactions, ddGTP, ddATP, ddTTP, and ddCTP were added to the above mix in concentrations of 50, 500 and 5000 μM each. Five units of polymerase 3173 mutant D49A were added and the reactions were incubated for 30 minutes at 70° C. Extension of the primer was detected by the ABI 310 Genetic Analyzer in Gene Scan mode. In this experiment, no inhibition of primer extension was detected, even at a 100-fold molar excess of chain terminator, suggesting a strong discrimination against the analogs by polymerase 3173 mutant D49A.

In a second experiment, incorporation was tested by detection of DNA synthesis using a double-strand specific fluorescent dye, Pico Green (Invitrogen). Unlabeled M13 primer (2 μM) was added to M13mp18 ssDNA (1.2 μM) in buffer containing 20 mM Tris-HCl, 10 mM (NH₄)₂SO₄, 10 mM KCl, 2 mM MgSO₄, 0.1% Triton X-100, pH 8.8 at 25° C., and 2 mM each of dGTP, dATP, dTTP and dCTP. In separate reactions, a mix of ddGTP, ddATP ddTTP, ddCTP (2 mM each) and a mix of the four acyNTPs (2 mM each) were added to extension reactions followed by DNA polymerase. As a control, identical reactions without added chain terminating analogs were also performed. Polymerase 3173 mutant D49A was tested and, for comparison, T7 DNA polymerase, which incorporates ddNTPs with very low discrimination, and Klenow fragment of E. coli polymerase I and VENT_{R} DNA polymerase (New England Biolabs), both of which have a higher discrimination, were also tested. Extension of the primer was detected by fluorescence of Pico Green dye. The results are shown in Table 5 below Inhibition of the polymerase 3173 mutant D49A enzyme by chain terminators was minimal.

TABLE 5
Incorporation Rates of Nucleotide Analogs Relative
to Incorporation Rates of Standard Nucleotides

| | 3173 D49A | T7 | Klenow | VENT_{R} |
|---|---|---|---|---|
| dNTPs | 100.0% | 100.0% | 100.0% | 100.0% |
| ddNTPs | 66.0% | 17.7% | 49.4% | 85.5% |
| acycloNTPs | 84.0% | 32.3% | 73.8% | 67.3% |

Based on alignment with family A DNA polymerases, amino acid 418 of the polymerase 3173 mutant D49A was mutated from phenylalanine to tyrosine. The mutant protein was expressed and the cells lysed and heat-treated at 70° C. for 10 minutes to inactivate host proteins. The polymerase 3173 mutant D49A/F418Y was tested for inhibition of radioactive nucleotide incorporation using chain terminating nucleotide analogs in the same mix as unlabeled deoxynucleotides. A reaction including 20 mM Tris-HCl, 10 mM (NH₄)₂SO₄, 10 mM KCl, 2 mM MgSO₄, 0.1% Triton X-100, pH 8.8 at 25° C., 0.25 mg/ml activated ct DNA, 40 μM each of dGTP, dATP, dTTP and dCTP and 0.1 μCi [α P-33] dCTP was used. In separate reactions both the D49A/F418Y mutant and purified polymerase 3173 mutant D49A were tested for inhibition by 4 mM each of ddNTPs and 4 mM each acycloNTPs. A control with no chain terminators was included. 50 μA reactions were incubated at 70° C. for 30 min. 15 μl of each reaction was spotted on DE81 paper, washed and counted, and units of activity were determined as described in Example 6. The degree of inhibition due to incorporation of dideoxy- and acyclo-nucleotides is shown in Table 6 below.

TABLE 6
Incorporation of Chain-Terminating Deoxynucleotides
Relative to Non-Chain-Terminating Deoxynucleotides

| | no terminators | ddNTPs | acyNTPs |
|---|---|---|---|
| 3173 D49A | 100.0% | 92.6% | 97.7% |
| 3173 D49A/F418Y | 100.0% | 0.8% | 1.1% |

The polymerase 3173 double mutant D49A/F418Y was also tested in the fluorescent primer extension assay described above. A 2× ratio of ddGTP:dGTP almost completely inhibited any extension. A 0.2× ratio of ddGTP:dGTP resulted in nearly complete inhibition of primer extension, with no extension continuing beyond the fourth G residue. Together, this data suggests that discrimination by the polymerase 3173 mutant D49A/F418Y against the chain terminating nucleotides that were tested is nearly zero.

### Example 18

### Isolation of Uncultured Viral Particles from a Second Thermal Spring

Viral particles were isolated from a water sample collected from a hot spring in Great Boiling Spring Park (N 40.652978 and W −119.351906; temperature 74° C.). Approximately two hundred liters of thermal water was filtered using a 100-kiloDalton (kD) molecular weight cut-off (mwco) tangential flow filter (A/G Technology, GE Healthcare, Piscataway, N.J.) and concentrated to 2 L. The resulting concentrate, containing viruses and microbes, was centrifuged to reduce numbers of microbial cells and filtered through a 0.2-μm tangential flow filter to further remove microbial cells. The viral fraction was further concentrated to 100 ml using a 100-kD tangential flow filter. Of the 100 ml of viral concentrate, 40 ml were further concentrated to 400 μl and transferred to SM buffer (0.1 M NaCl, 8 mM MgSO4, 50 mM Tris-HCl, pH 7.5) by filtration in a 30-kDa mwco spin filter (Centricon, Millipore, Billerica, Mass.).

### Example 19

### Isolation of Viral DNA

Serratia marcescens endonuclease (10 U) (Sigma-Aldrich, St. Louis, Mo.) was added to the viral preparation described in Example 1 to remove non-encapsidated (non-viral) DNA. The reaction was incubated for 30 min at 23° C. Ethylenediaminetetraacetic acid (EDTA) (20 mM) and sodium dodecyl sulfate (SDS) (0.5%) were then added. To isolate viral DNA, proteinase K (100 U) was added, and the reaction was incubated for 3 hours at 56° C. Sodium chloride (0.7 M) and cetyltrimethylammonium bromide (CTAB) (1%) were then added. The DNA was extracted once with chloroform, once with phenol, once with a phenol:chloroform (1:1) mixture, and again with chloroform. The DNA was precipitated with 1 ml of ethanol and washed with 70% ethanol. The yield of DNA was 20 ng.

### Example 20

### Construction of a Viral DNA Library

The viral DNA purified in Example 19 was amplified using “REPLI-G”-brand DNA amplification kit (Qiagen, Valencia, Calif.) according to the manufacturer's recommendations. The amplification products were treated with S1 nuclease and sheared using a “HYDROSHEAR”-brand DNA shearing device (Genomic Solutions, Inc. Ann Arbor, Mich.). To create a viral DNA library, the sheared nucleic acid was inserted into the cloning site of the “pETITE”-brand vector (Lucigen, Middleton, Wis.). The vectors with inserts were transformed into “E.CLONI”-brand 10G electrocompetent cells (Lucigen, Middleton, Wis.).

### Example 21

### Screening Viral Libraries by Functional Activity

Approximately twenty eight hundred clones from the library described in Example 20 were screened by testing for thermostable DNA polymerase activity. Each clone was tested by culturing the clones, lysing the cells enzymatically, exposing the cell lysates to 70° C. for 10 minutes to inactivate the host DNA polymerase activities, and assaying for DNA polymerase activity at 70° C. using the assay described in Example 6. Twelve clones tested positive. Preliminary results suggested that eleven of these clones were highly similar to one another in amino acid sequence. This high similarity group is referred to herein as the “74-like” polymerase family in reference to Clone 74, the first of this family that was discovered. Only eight of these eleven were analyzed further. Seven of the eight 74-like polymerase clones and a unique clone, Clone 347, were confirmed to have polymerase activity by the DNA polymerase assay described in Example 6. The results are shown in Table 7. In each case the counts adhering to the filter in the absence of added DNA polymerase were lower than 500.

TABLE 7
DNA Polymerase Activity Assays on Functionally-
Screened DNA Polymerase Clones

| Clone | Polynucleotide | Polypeptide | Counts on Filter |
|---|---|---|---|
| 347 | SEQ ID NO: 30 | SEQ ID NO: 31 | 18710 |
| 74 | SEQ ID NO: 32 | SEQ ID NO: 33 | 47398 |
| 2783GBS | SEQ ID NO: 34 | SEQ ID NO: 35 | 11513 |
| 1160 | SEQ ID NO: 36 | SEQ ID NO: 37 | 139291 |
| 1440 | SEQ ID NO: 38 | SEQ ID NO: 39 | not determined |
| 1128 | SEQ ID NO: 40 | SEQ ID NO: 41 | 16383 |
| 1753 | SEQ ID NO: 42 | SEQ ID NO: 43 | 141358 |
| 1773 | SEQ ID NO: 44 | SEQ ID NO: 45 | 124166 |
| 1937 | SEQ ID NO: 46 | SEQ ID NO: 47 | 70335 |

The sequences of the inserts of nine of the positive clones, including eight of the 74-like polymerase clones and the unique Clone 347, were determined by standard methods. These sequences were conceptually translated and compared to the database of non-redundant protein sequences in GenBank (National Center for Biotechnology Information [NCBI]) using the BLASTx program (NCBI). The sequence identification numbers of the respective inserts and their conceptual translations are shown in Table 7. The translated sequences were also compared to one another using the ClustalW program to determine similarity among the clones (FIGS. 7A-E). A region of overlap was detected among the eight 74-like clones, which shared greater than 97% sequence identity to one another over at least a portion of their sequences (see position 461 onward of the alignment depicted in FIGS. 7A-E). This family appeared to encode a polyprotein of at least 998 amino acids, of which only the carboxy-terminal half had sequence similarity to known pol genes. As shown in FIGS. 7A-E, the eight different 74 family clones varied in the amount of coding sequence in the amino terminus, but all included the complete carboxy-terminal half of the open reading frame (ORF). For example, Clone 1773 of the 74-like family encoded an uninterrupted ORF of 998 amino acids. Clone 2783 encoded an ORF of 538 amino acids that was nearly identical to the carboxy terminal half of 1773. Notwithstanding its apparent truncation, Clone 2783 encoded a fully functional DNA polymerase. Despite significant differences in sizes of the ORFs encoded by the inserts of Clones 1160, 1753, 1773, and 1937, SDS PAGE indicated that expression of all the clones resulted in thermostable proteins of about 55 kD. This is apparently due to self cleavage of the putative polyprotein in a biochemical reaction analogous to examples previously described in the art. Thus, the polypeptides described herein (and polynucleotides encoding the polypeptides) can be truncated N-terminally to a position corresponding to position 461 of the alignment depicted in FIGS. 7A-E and still comprise an active DNA polymerase.

Based on the alignment shown in FIGS. 7A-E, nucleotide and protein consensus sequences were determined using ClustalW. Nucleotide and protein full-length consensus sequences of the eight 74-like clone sequences are included herein as SEQ. ID. NOS: 60 and 61, respectively. Nucleotide and protein consensus sequences of the truncated sequence shown to have polymerase activity, as described above, are included herein as SEQ. ID. NOS: 62 and 63, respectively.

The twelfth clone, Clone 347, shared no similarity to this group or to any known DNA polymerase, although it shared weak similarity to presumptive crenarchaeal viral protein of unknown function described below. The 1776-nucleotide gene (SEQ ID NO:30) of Clone 347 encoded a 391-amino acid protein (SEQ ID NO:31) with DNA polymerase activity.

### Example 22

### Identification and Characterization of Motif A and Motif B in Viral DNA Polymerases of the Invention

DNA polymerases have several motifs that are critical to polymerase function. Certain Family A-type viral DNA polymerases of this invention can be defined by sequence variations in such critical motifs. These sequence variations are common among the viral DNA polymerases of this invention but are unique compared to all other known DNA polymerases.

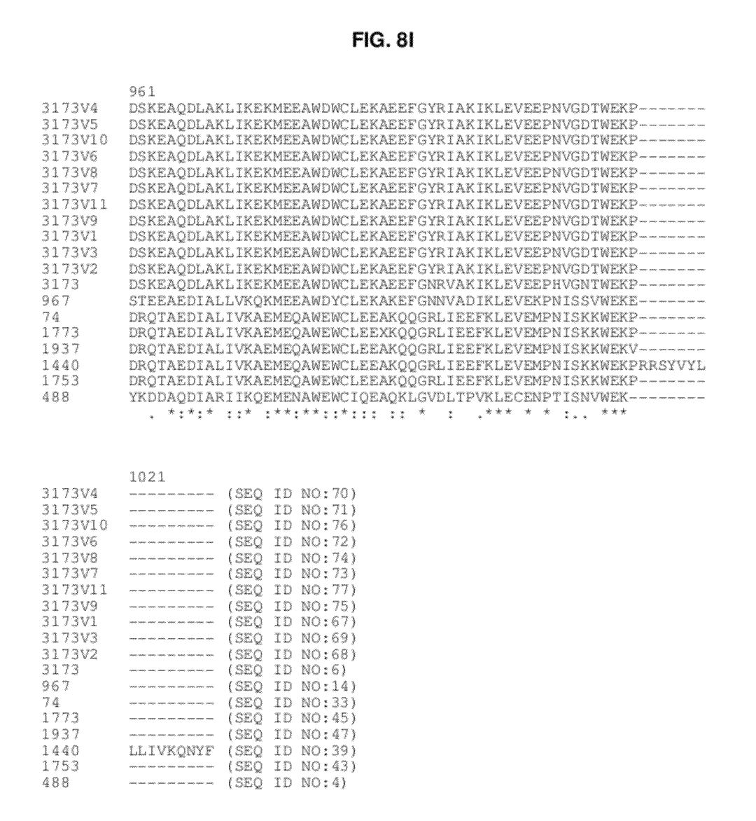

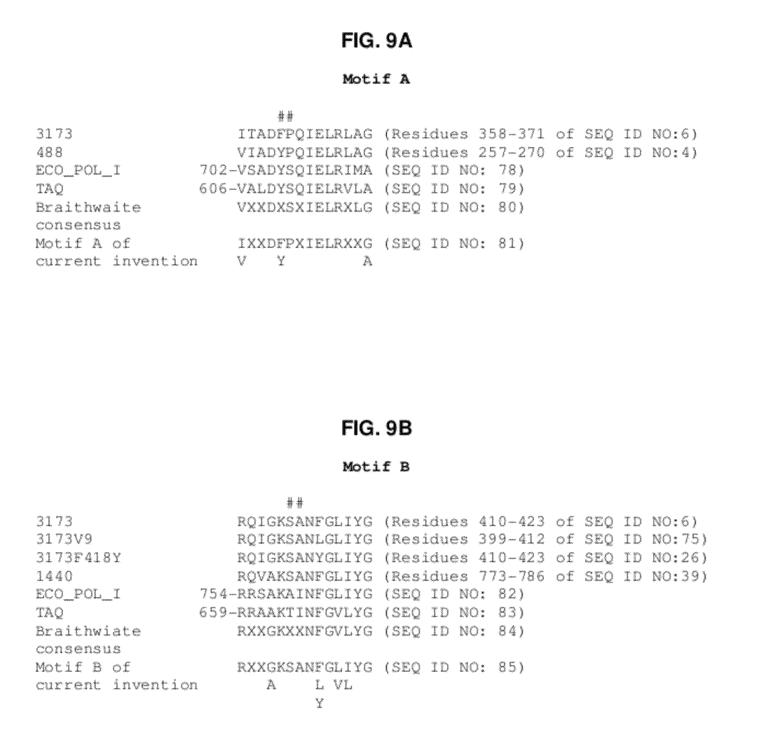

In 1991 and 1993, Braithwaite and Ito (Braithwaite D K et al. Nucleic Acids Res. 1993 21(4):787-802; and Ito J et al. Nucleic Acids Res. 1991 19(15):4045-57) published a series of alignments of DNA polymerase primary sequences that allowed four key observations relevant to the present invention. First, known DNA polymerase sequences could be grouped into one of four families (A, B, C and X). Second, viral DNA polymerases are highly divergent from cellular DNA polymerases. Third, DNA polymerases of all known viruses except Phages T7, T5, Spo1 and Spo2 are of the Family B-type. Fourth, certain specific domains are highly conserved. Relevant to this invention are the highly conserved consensus sequences, VXXDXSXIELRXLG (SEQ ID NO:80) and RXXGKXXNFGVLYG (SEQ ID NO:84), wherein X is unspecified. These consensus sequences were referred to in later publications as Motifs A and Motif B, respectively (FIGS. 9A and 9B).

These findings have been supported and extended by more recent data. The number of polymerase families has increased to include Families D and Y since the Braithwaite and Ito publications, but most of the newly discovered DNA polymerases fall into one of the earlier four families. Virtually all of the viral DNA polymerases discovered since the Braithwaite and Ito publications have aligned most strongly with Family B. Among family A DNA polymerases, three regions of highest sequence similarity are commonly recognized and referred to in the art as Motifs A, B and C. Based on subsequent work, the basis of conservation has been ascribed to the highly critical and fundamental roles of these motifs in the overall function of the DNA polymerases. The amino acids in these motifs have demonstrated roles in contacting the template or nucleotides or in catalytic activity of the enzymes. Alteration of amino acid residues in Motifs A and B has a measurable impact on the function and utility of the DNA polymerases.

Motif A spans the bend between Beta-strand 9 and the L-helix of Family A DNA polymerases. This region comprises the junction between the palm and the fingers of the DNA polymerase molecule and is involved with binding of the template DNA (Li et al. EMBO J. 1998 17(24):7514-25). The aspartate in position 4 of Motif A (numbering based on Motif A sequences shown in FIG. 9A) is believed to be responsible for chelating divalent cations, is a member of the DNA polymerase catalytic triad, and is, hence, invariant in Family A Pols. Mutagenesis of Motif A has delineated the function of other specific amino acid residues. Substitution of the alanine at the second position in Taq Motif A (SEQ ID NO: 79; see FIG. 9A) to threonine or serine has been shown to increase use of RNA as a template (i.e., in reverse transcription) (Vichier-Guerre et al. Angew Chem Int Ed Engl. 2006 45(37):6133-7). The isoleucine in the eighth position has been shown to be critical for insertion fidelity (Patel et al. J Biol Chem. 2001 276(7):5044-51).

Motif B is also critical to the utility of DNA polymerase. This motif spans the O-helix in the fingers of the polymerase structure that is associated with binding of the nucleotide prior to incorporation into the nascent strand. Amino acids arginine, lysine, and phenylalanine (residues 1, 5 and 9 of the Taq Motif B (SEQ ID NO:83) as shown in FIG. 9B) all bind the nucleotides in the closed structure during synthesis, while the tyrosine (position 13) of the Taq Pol binds nucleotide in the open configuration between rounds of incorporation (Li et al. Protein Sci. 2001 10(6):1225-33). The tyrosine of Motif B in E. coli and Taq polymerases (SEQ ID NOS: 82 and 83; see FIG. 9B) has been altered to increase incorporation of chain terminating nucleotides and, thereby, improve functionality as a DNA sequencing reagent (Tabor et al. Proc Natl Acad Sci USA 1995 92(14):6339-43). Alanine and threonine (positions 4 and 6 of Taq Motif B) have been shown to be important for fidelity. The threonine residue in the Taq polymerase appears important to correct insertion and extension, as substitution with proline negatively affects fidelity at both levels (Tosaka et al. J Biol Chem. 2001 276(29):27562-7). The alanine has been shown to be important for correct discrimination against incorrect nucleotides (Ogawa et al. Mutat Res. 2001 485(3):197-207). The phenylalanine, isoleucine, alanine in Motif B are all important to fidelity. Furthermore, the residues in the O-helix adjacent to Motif B have an important effect on strand displacement and initiation at nicks (Singh et al. J Biol Chem. 2007 282(14):10594-604) and in stabilization of the pre-polymerase ternary structure (Srivastava et al. Biochemistry 2003 42(13):3645-54). These activities impact the utility of DNA polymerases in amplification and sequencing.

The viral polymerases of the present invention were isolated from three different hot springs hundreds of miles apart over a span of about six years (Table 8). These viral polymerases were identified by different criteria in metagenomes isolated from four separate sampling expeditions. Polymerases 3173 and 967 were isolated from a hot spring in Yellowstone National Park by BLASTx analysis based on similarity to known polymerase sequences. Polymerases 74, 1440, 1753, 1773, 1937 were among eleven highly related polymerases isolated from a Nevada hot spring in a screen for DNA polymerase activity. Polymerase 488 was isolated from Little Hot Creek in Long Valley, Calif. using BLASTx analysis. Polymerases designated V6, V7, V8, V9, V12, V1, V2, V4, V5, V10, V11 were isolated by PCR amplification using primers specific for polymerase 3173 from the same hot spring as 3173, but in a sample isolated four years later.

TABLE 8
Sources of Viral Polymerases of the Invention

| Viral | | | Source of | Year |
|---|---|---|---|---|
| Pol | Polynucleotide | Polypeptide | Sample | Collected |
| 3173 | SEQ ID NO: 5 | SEQ ID NO: 6 | OHS | 2003 |
| 967 | SEQ ID NO: 13 | SEQ ID NO: 14 | OHS | 2003 |
| 74 | SEQ ID NO: 32 | SEQ ID NO: 33 | GBS | 2008 |
| 1440 | SEQ ID NO: 38 | SEQ ID NO: 39 | GBS | 2008 |
| 1753 | SEQ ID NO: 42 | SEQ ID NO: 43 | GBS | 2008 |
| 1773 | SEQ ID NO: 44 | SEQ ID NO: 45 | GBS | 2008 |
| 1937 | SEQ ID NO: 46 | SEQ ID NO: 47 | GBS | 2008 |
| 488 | SEQ ID NO: 3 | SEQ ID NO: 4 | LHC | 2001 |
| V1 | — | SEQ ID NO: 67 | OHS | 2007 |
| V2 | — | SEQ ID NO: 68 | OHS | 2007 |
| V3 | — | SEQ ID NO: 69 | OHS | 2007 |
| V4 | — | SEQ ID NO: 70 | OHS | 2007 |
| V5 | — | SEQ ID NO: 71 | OHS | 2007 |
| V6 | SEQ ID NO: 64 | SEQ ID NO: 72 | OHS | 2007 |
| V7 | SEQ ID NO: 65 | SEQ ID NO: 73 | OHS | 2007 |
| V8 | SEQ ID NO: 66 | SEQ ID NO: 74 | OHS | 2007 |
| V9 | — | SEQ ID NO: 75 | OHS | 2007 |
| V10 | — | SEQ ID NO: 76 | OHS | 2007 |
| V11 | — | SEQ ID NO: 77 | OHS | 2007 |

OHS = Octopus Hot Spring, Yellowstone National Park
GBS = Great Boiling Spring, Gerlach, Nevada
LHC = Little Hot Creek, Long Valley, CA

The viral polymerases of the present invention vary by as much as 60% at the amino acid level (Table 9). However, the isolated viral polymerases share two notable sequence signatures at sites that align to sequences corresponding to Motifs A and B as described by Braithwaite and Ito (see FIGS. 9A and 9B). Specifically, Motif A of the isolated viral polymerases can be defined by the sequence (I/V)XXD(F/Y)PXIELRXX(G/A) (X denoting any amino acid) (SEQ ID NO:81). Motif B of the viral polymerases can be defined by the sequence RXX(G/A)KSAN(F/L/Y)G(L/V)(I/L)YG (SEQ ID NO:85).

TABLE 9
Amino Acid Sequence Identities (in Percent Identity) of the Family A Thermophilic Viral DNA Polymerases

| | 3173 | 967 | 74 | 1440 | 1753 | 1773 | 1937 | 488 | V6 | V7 | V8 | V9 | V1 | V3 | V2 | V4 | V5 | V10 | V11 |
|---|---|---|---|---|---|---|---|---|---|---|---|---|---|---|---|---|---|---|---|
| 3173 | 100 | | | | | | | | | | | | | | | | | | |
| 967 | 82 | 100 | | | | | | | | | | | | | | | | | |
| 74 | 45 | 39 | 100 | | | | | | | | | | | | | | | | |
| 1440 | 45 | 39 | 99 | 100 | | | | | | | | | | | | | | | |
| 1753 | 45 | 39 | 97 | 98 | 100 | | | | | | | | | | | | | | |
| 1773 | 45 | 39 | 99 | 96 | 98 | 100 | | | | | | | | | | | | | |
| 1937 | 44 | 47 | 98 | 98 | 97 | 98 | 100 | | | | | | | | | | | | |
| 488 | 46 | 46 | 56 | 56 | 57 | 56 | 56 | 100 | | | | | | | | | | | |
| V6 | 94 | 80 | 45 | 45 | 45 | 45 | 45 | 45 | 100 | | | | | | | | | | |
| V7 | 94 | 80 | 45 | 45 | 45 | 44 | 44 | 45 | 99 | 100 | | | | | | | | | |
| V8 | 93 | 80 | 45 | 45 | 45 | 44 | 44 | 45 | 99 | 98 | 100 | | | | | | | | |
| V9 | 94 | 80 | 45 | 45 | 45 | 45 | 45 | 45 | 99 | 99 | 98 | 100 | | | | | | | |
| V1 | 93 | 80 | 45 | 45 | 45 | 44 | 44 | 45 | 99 | 98 | 98 | 98 | 100 | | | | | | |
| V3 | 94 | 80 | 45 | 45 | 45 | 45 | 45 | 45 | 99 | 99 | 98 | 99 | 98 | 100 | | | | | |
| V2 | 94 | 80 | 45 | 45 | 45 | 45 | 45 | 45 | 99 | 99 | 98 | 99 | 98 | 100 | 100 | | | | |
| V4 | 94 | 80 | 45 | 45 | 45 | 45 | 45 | 46 | 99 | 99 | 99 | 99 | 99 | 99 | 99 | 100 | | | |
| V5 | 94 | 80 | 45 | 45 | 45 | 45 | 45 | 46 | 99 | 99 | 99 | 99 | 99 | 99 | 99 | 100 | 100 | | |
| V10 | 94 | 80 | 45 | 45 | 45 | 45 | 45 | 46 | 99 | 99 | 99 | 99 | 99 | 99 | 99 | 100 | 100 | 100 | |
| V11 | 94 | 80 | 45 | 45 | 45 | 44 | 44 | 45 | 99 | 100 | 98 | 99 | 98 | 99 | 99 | 99 | 99 | 99 | 100 |

With reference to Motif A of the viral polymerases, the phenylalanine in position 5 and the proline in position 6 (denoted by ## in FIG. 9A) are unique to the viral DNA polymerases of the invention and are shared by all but one of the isolated viral polymerases.

Positions 5 and 6 of Motif A are important for the activity of the viral polymerases. First, DNA synthesis involves the opening and closing of the “palm” and “fingers” of DNA polymerase. The amino acids at positions 5 and 6 of Motif A form a “hinge” between the palm and fingers. The inclusion of proline at position 6 of the Family A viral polymerases is unexpected, as it is widely understood that proline restricts the flexibility of a protein's structure and, when placed near an active site, alters enzyme activity. This is particularly important as the proline at position 6 of Motif A is two residues away from the aspartate residue, which is a member of the DNA polymerase catalytic triad. Second, positions 5 and 6 of Motif A are identified in the DNA polymerase structure as providing important contacts with template DNA (Li et al. Protein Sci. 2001 10(6):1225-33). Polymerase 3173 and its variants are distinguished from virtually all other Family A polymerases in their ability to efficiently use an RNA template in addition to a DNA template. RNA and DNA differ from one another by a hydroxyl group. It is logical that the reverse transcriptase activity of the 3173 polymerase is due to the substitution of the aromatic phenylalanine for the hydroxyl tyrosine, thereby allowing use of an RNA template. This is analogous to the substitution of phenylalanine (position 9 of Motif B) for tyrosine which allows use of dideoxynucleotides (see Examples above), the latter of which differ from deoxynucleotides by absence of a hydroxyl group.

With reference to Motif B, the serine/alanine dipeptide at positions 6 and 7 is shared by all the Family A viral polymerases of the present invention, but is unique with respect to all other known DNA polymerases (see ## in FIG. 9B). The alanine in position 7 is particularly distinguishing. Alanine at position 7 appears to be otherwise absent in nature. In addition, this amino acid is not present in prior functional mutants. Suzuki et al. (Suzuki et al. Proc Natl Acad Sci USA 1996 93(18):9670-5) randomly mutagenized Motif B of Taq polymerase. Among the functional mutants, they recovered 61 different mutations affecting ten of the 13 positions (R, K, and G at positions 1, 5 and 10 were invariant). Twelve of these independent mutations affected position 7. However, substitution of alanine for the wild-type isoleucine was not found at position 7 in functional mutants. The polymerases described herein comprising the alanine of position 7, however, all show functional DNA polymerase activity. Furthermore, the residues at positions 6 and 7 of Motif B are likely to be important to the utility of DNA polymerase since this motif spans the O-helix in the fingers of the Taq structure, which, as noted above, is critical to binding of deoxynucleotide triphosphates prior to incorporation and strand displacement.

### Example 23

### Identification of Pol I Genes in Sequenced Microbial and Viral Genomes

The sequences of three cultivated microbes, Dictyoglomus turgidum, strain DSM 6724; Sulfurihydrogenibium sp., strain YO3AOP1; and Hydrogenobaculum sp., strain Y04AAS1, were determined in conjunction with the U.S. Department of Energy, Joint Genome Institute (Walnut Creek, Calif.). These genomes have since been deposited in GenBank (Accession Nos. CP001251, CP001080, and CP001130). The pol I genes of each of these microbes, as well as the pol I gene of Dictyoglomus thermophilum H-6-12, previously deposited in GenBank (Accession No. NC_{—}011297) were identified in the genomic sequences by sequence similarity to numerous pol I genes of known microbes. These genes were amplified by PCR, inserted in an expression vector, and sequenced. The nucleotide and protein sequences of the polymerase derived from Dictyoglomus turgidum (“Dtu DNA Pol I”) were SEQ. ID. NOS: 52 and 53, respectively. The nucleotide and protein sequences of the polymerase derived from Dictyoglomus thermophilum (“Dth DNA Pol I”) were SEQ. ID. NOS: 54 and 55, respectively. The nucleotide and protein sequences of the polymerase derived from Sulfurihydrogenibium sp. (“Sye DNA Pol I”) were SEQ. ID. NOS: 50 and 51, respectively. The nucleotide and protein sequences of the polymerase derived from Hydrogenobaculum sp. (“Hac DNA Pol I”) were SEQ. ID. NOS: 48 and 49, respectively.

Another gene; referred to herein as “SSV dnaA,” was identified in the Sulfolobus viral genome (GenBank Accession No. SSV-1p01 NP_{—}039777) based on weak similarity (E value=0.15) to the 347 protein. This gene was previously annotated as a “hypothetical protein.” To our knowledge, this gene has never previously been expressed, and no function has ever been demonstrated in relation to the expressed protein. The nucleotide sequence of the open reading frame and the protein sequences are SEQ ID. NOS. 56 and 57, respectively. The SSV dnaA gene was transferred to an expression vector, expressed as described below, and is being tested for primase activity. It is predicted that SSV dnaA polymerase has primase activity. As is known in the art, primase is a subclass of RNA polymerase enzymes that initiates genome replication by catalyzing synthesis of an RNA polynucleotide primer on a DNA template in the absence of any other primer.

### Example 24

### Expression of DNA Polymerase Genes

The polymerase genes described in Example 23 were expressed in E. coli BL21(DE3) competent cells (Lucigen, Middleton, Wis.) or a similar E. coli strain. The proteins were extracted, heated to 70° C. for 10 minutes, and tested for DNA polymerase activity using the DNA polymerase assay described in Example 6. Each protein was confirmed to have polymerase activity.

### Example 25

### Polymerase Chain Reaction Using Dtu Polymerase

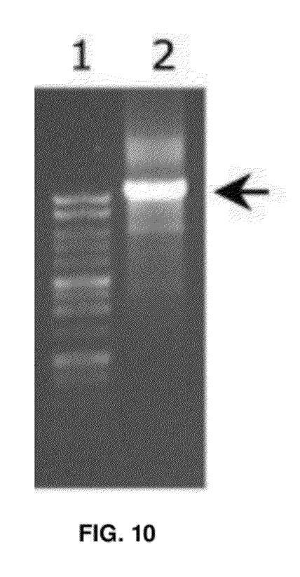

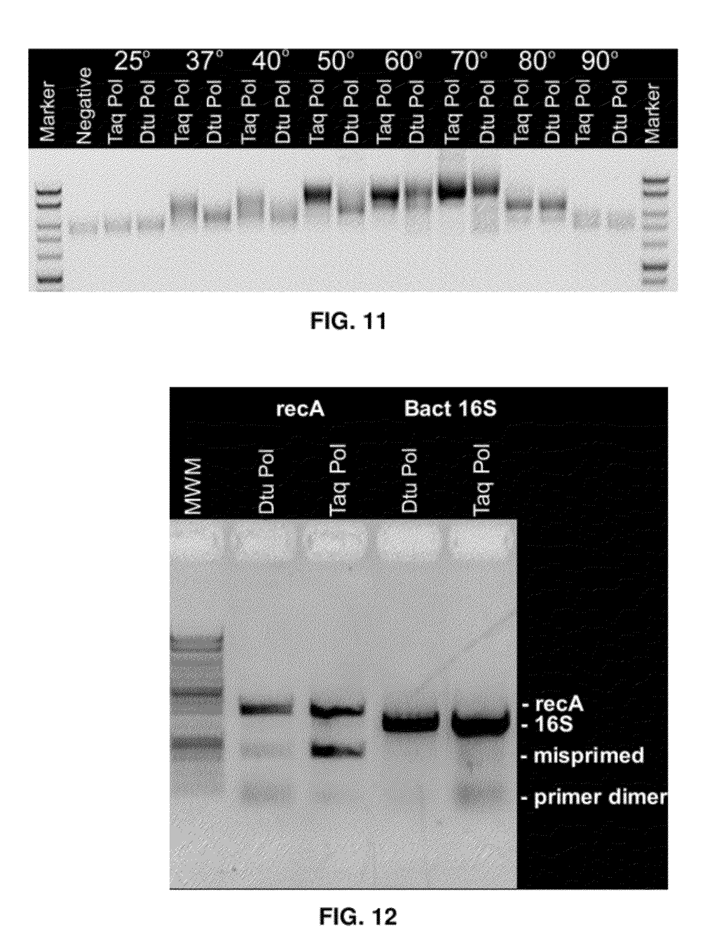

To verify its utility in PCR, Dtu Pol was used to amplify a 10-kb product from phage lambda genomic DNA. The polymerase chain reaction included 20 mM Tris-HCl (pH 8.8 at 25° C.), 10 mM (NH₄)₂SO₄, 10 mM KCl, 2 mM MgSO₄, 0.1% Triton X-100, 15% sucrose, 0.2 mM each of dGTP, dATP, dTTP and dCTP, 10 ng lambda DNA (GenBank Accession No. NC_{—}001416), 0.08 μM of each of two primers (SEQ. ID. NOS: 29 and 30), and 5 units of Dtu Pol. After thermal cycling (one cycle of 94° C. for 2 minutes, 25 cycles of 94° C. for 15 seconds, 60° C. for 15 seconds, and 72° C. for 10 minutes, followed by one cycle at 72° C. for 10 minutes), reactions were resolved using agarose gel electrophoresis. The results are shown in FIG. 10. Lane 1 shows a molecular weight marker ranging from 250 to 10,000 bp. Lane 2 shows the amplification product. The arrow indicates the location of the expected amplification product. As shown in FIG. 11, the Dtu Pol was incubated with a primed M13 template in conditions that promote extension of the primer. Reduced activity was observed below about 60° C. In FIG. 12, The Dtu was compared to Taq polymerase for mispriming using two primer/target sets with a known propensity for generating misprimed products. Each enzyme was used under the conditions described above. The Dtu polymerase was associated with notably reduced generation of secondary, nontarget product.

The invention has been described with reference to various specific embodiments and techniques. However, it should be understood that many variations and modifications may be made while remaining within the spirit and scope of the invention.

We claim:

1. A substantially purified polymerase having an amino acid sequence comprising SEQ ID NO:6, sequence variants at least about 85% identical to SEQ ID NO:6, or fragments of SEQ ID NO:6 having polymerase activity.

2. The polymerase of claim 1, wherein the polymerase
comprises aspartate at a position corresponding to position 49 of SEQ ID NO:6 and glutamate at a position corresponding to position 51 of SEQ ID NO:6; and
exhibits exonuclease activity.

3. The polymerase of claim 1, wherein the polymerase:
comprises a residue other than aspartate at a position corresponding to position 49 of SEQ ID NO:6, a residue other than a glutamate at a position corresponding to position 51 of SEQ ID NO:6, or a residue other than aspartate at a position corresponding to position 49 of SEQ ID NO:6 and a residue other than a glutamate at a position corresponding to position 51 of SEQ ID NO:6; and
substantially lacks exonuclease activity.

4. The polymerase of claim 1, wherein the polymerase:
comprises a residue other than phenylalanine at a position corresponding to position 418 of SEQ ID NO:6; and
has a relative incorporation efficiency of nucleotide analogs that is at least 10% of the incorporation efficiency of standard deoxynucleotides.

5. The polymerase of claim 1, wherein the polymerase:
comprises a residue other than aspartate at a position corresponding to position 49 of SEQ ID NO:6, a residue other than a glutamate at a position corresponding to position 51 of SEQ ID NO:6, or a residue other than aspartate at a position corresponding to position 49 of SEQ ID NO:6 and a residue other than a glutamate at a position corresponding to position 51 of SEQ ID NO:6;
comprises a residue other than phenylalanine at a position corresponding to position 418 of SEQ ID NO:6; and
substantially lacks exonuclease activity and has a relative incorporation efficiency of nucleotide analogs that is at least 10% of the incorporation efficiency of standard deoxynucleotides.

6. The polymerase of claim 1 wherein the polymerase exhibits an activity selected from the group consisting of reverse transcriptase activity and strand displacement activity.

7. The polymerase of claim 1 having an amino acid sequence comprising SEQ ID NO: 6 or sequence variants at least about 90% identical thereto.

8. The polymerase of claim 1 having an amino acid sequence comprising SEQ ID NO:6 or sequence variants at least about 95% identical thereto.

9. The polymerase of claim 1 having an amino acid sequence comprising SEQ ID NO:6, SEQ ID NO.25, SEQ ID NO:26, or SEQ ID NO:27.

10. The polymerase of claim 1 wherein the amino acid sequence includes a motif selected from the group consisting of:
a first motif having sequence X₁X₂X₃DX₄PX₅IELRX₆X₇X₈, wherein:
X₁ is I or V;
X₄ is F or Y;
X₈ is G or A; and
X₂, X₃, X₅, X₆, and X₇ are any amino acid (SEQ ID NO: 81); and
a second motif having sequence RX₉X₁₀X₁₁KSANX₁₂GX₁₃X₁₄YG, wherein:
X₁₁ is G or A;
X₁₂ is F, L, or Y;
X₁₃ is L or V;
X₁₄ is I or L; and
X₉ and X₁₀ are any amino acid (SEQ ID NO: 85).

11. The DNA polymerase of claim 10 wherein the sequence X₁X₂X₃DX₄PX₅IELRX₆X₇X₈ of the first motif is selected from the group consisting of ITADFPQIELRLAG (residues 358-371 of SEQ ID NO:6) and VIADYPQIELRLAG (residues 257-270 of SEQ ID NO:4).

12. The DNA polymerase of claim 10 wherein the sequence RX₉X₁₀X₁₁KSANX₁₂GVLYG of the second motif is selected from the group consisting of RQIGKSANFGLIYG (residues 410-423 of SEQ ID NO:6), RQIGKSANLGLIYG (residues 399-412 of SEQ ID NO:75), RQIGKSANYGLIYG (residues 410-423 of SEQ ID NO:26), and RQVAKSANFGLIYG (residues 773-786 of SEQ ID NO:33).

13. The polymerase of claim 1 comprising a motif consisting of KSANFGLIYG (residues 414-423 of SEQ ID NO:6) or KSANYGLIYG (residues 414-423 of SEQ ID NO:26).

14. A method of producing the polymerase of claim 1 comprising expressing an isolated polynucleotide encoding the polymerase of claim 1.

15. The method of claim 14, wherein the isolated polynucleotide comprises the sequence of SEQ ID NO: 5.

16. A method of polymerizing a polynucleotide comprising contacting a template of the polynucleotide with the polymerase of claim 1 in the presence of a compound selected from the group consisting of a nucleotide and a nucleotide analog under conditions sufficient to promote synthesis of a copy or complement of the template.

17. The method of claim 16, wherein the conditions comprise maintaining substantially isothermal conditions.

18. The method of claim 16, wherein the conditions comprise thermocycling and include at least one set of primers.

19. The method of claim 16, wherein the conditions exclude manganese.

20. The method of claim 16, wherein the conditions comprise the presence of a nick-inducing agent and exclude primers.

21. The method of claim 16, wherein the template is RNA.

22. The method of claim 16, wherein the template is DNA.

23. The method of claim 16, wherein the template comprises an amplification-resistant sequence.

24. The method of claim 16, wherein the nucleotide analog is a chain-terminating analog.


SEQUENCE LISTING (85 sequences)

SEQ ID NO: 1 - DNA, 1743 bp, Unknown
  Clone 3063 from uncultured newly isolated virus
atgaaggtgagctttgaatacatcacatctccaaaatcccttgccaagtgggaagggagctttaaggatataccctttttgtatattgatacggaaacggtgggagacagcaccataaggctcgtccaattgggaactgaaaaagacatactccttttggacctattcgagcttggtgatgtaggaattaactttttaaaggaactgctttcccagaagggtatagtgggtcataatctaaagtttgacctgaagtatctacttggctatggaatagagccctacgcagtctttgacaccatgatcgccagtcagctgttgggggactccgacaggcactcccttcagaaattagccatgcagtatttgggagaggtcatagacaagagccttcagctttccaactggggctcctcaaggctctcaaaggaacagttagaatatgccgccctggatgtggatgtagtcagaaggctctttccactgctccttgagaggttaaacagtcttacaccgatggtggaggaaaaccttcttaaaaccaggaccgcaaaggtctttgggctaaaaaaccccatcgccatagtggaaatggcttttgttcaggaggtggcaaagcttgaaagaaacgggctcccggtggatgtggaagaactggaaaggcttgtaaaggagctttcaaaggagcttcaaaaaagggtgatggactttttagtcaaatacagaacggaccccatgtctcccaaacaggtgggagagcttttggtcaaaaagtttggcttgaaccttccaaaaacagaaaagggcaacatatccaccgatgacaaatacttggcggaacacatagaaaaccctgcggtaagagaacttttgaagataagagagataaaaaagaacttggacaagcttgaggagattaaggatggtttgagggggaaaagggtatatccagagttcaagcagataggtgcaataaccgggcgaatgtcctccatgaaccccaacgtgcagaacattccaaggggcctaagaagaatctttaaggcggaggaaggaaatgtttttgtgatagcggacttttctcaaatagagctgagaatcgccgcagagtacgtaaacgatgagagtatgataaaggtatttagggaagggagggatatgcacaaatacactgccagcgtgctcttggggaaaaaggaggaagaaattacaaaggaagagaggcagttggcaaaggcggtaaattttgggctcatatacggcatatccgcaaagggtttggcagaatacgcttactcttcctacggcatagccctttcccttgcagaagcggagaaaataagggcaagattttttgaacacttcagaggctttaaggattggcacgaaagagttaagaaagaattaagggaaaaaggtaaatcagagggttataccttgcttggcagaagatacaccgcccacaccttcccagacgcggtcaattatcccatacagggaactggtgcggacctcttaaaactctctgtgctcatatttgacgcagaggtcagaagggaaaacatcaaagcccgtgtgataaacttggtgcatgacgagatagtggtggaatgtcccatggaggagggagaaaggactgcggagcttttggagagggctatgaaaagggctggtgggattatactaaagaaggtgcctgtggaagtagagtgtgtgataaaggagaggtgggaaaaggaataa

SEQ ID NO: 2 - protein, 580 aa, Unknown
  Clone 3063 from uncultured newly isolated virus
MKVSFEYITSPKSLAKWEGSFKDIPFLYIDTETVGDSTIRLVQLGTEKDILLLDLFELGDVGINFLKELLSQKGIVGHNLKFDLKYLLGYGIEPYAVFDTMIASQLLGDSDRHSLQKLAMQYLGEVIDKSLQLSNWGSSRLSKEQLEYAALDVDVVRRLFPLLLERLNSLTPMVEENLLKTRTAKVFGLKNPIAIVEMAFVQEVAKLERNGLPVDVEELERLVKELSKELQKRVMDFLVKYRTDPMSPKQVGELLVKKFGLNLPKTEKGNISTDDKYLAEHIENPAVRELLKIREIKKNLDKLEEIKDGLRGKRVYPEFKQIGAITGRMSSMNPNVQNIPRGLRRIFKAEEGNVFVIADFSQIELRIAAEYVNDESMIKVFREGRDMHKYTASVLLGKKEEEITKEERQLAKAVNFGLIYGISAKGLAEYAYSSYGIALSLAEAEKIRARFFEHFRGFKDWHERVKKELREKGKSEGYTLLGRRYTAHTFPDAVNYPIQGTGADLLKLSVLIFDAEVRRENIKARVINLVHDEIVVECPMEEGERTAELLERAMKRAGGIILKKVPVEVECVIKERWEKE

SEQ ID NO: 3 - DNA, 1461 bp, Unknown
  Clone 488 from uncultured newly isolated virus
gcggttgggacttggattacgaccttacaaaaacttggctttacatatgaagaacttgaagacaaggaagttttagatttgctttcaatagcaagattagtattaccagaaagatttaaagagaatggttttagtttggatgttgtgttgaaggaagtgttaggtattgattataaatttgataaaaagacaataagaaaaacatttacaccgcttttgatgacacaagaacaattagagtatatagcatctgatgtaatctacttgccagctttaaaagagaaacttgatgaaaagtttaataaaagactatggctaccttacatcttggacatggaagcaacaaaaattttagcagaagtgtctaacaatggtatgccatttcttaaagaaaaagcaaaagaagagcttagcagattaagcaaggaattagaaggacttagaaaagagcttggttttaatccaaactctccaaaagaaactcaaaaagttttaaacacaccagatacaagcgaagcaactctaatgaagttgataattagtaattcaagcaaaaaagctattgctgaaaaagttattcaagcaagaaaaatacaaaaagtaatagcaatgattaacaagtaccttaactatgatagagtaaaaggcacattctggactacaacagcgccatcaggtagaatgtcttgtgataaagaaaatttacaacaaataccaagaagtataagatatttgtttggctttgatgaaaactcagataaaacattagttatagcagattatccacaaatagaactaagacttgcaggtgtgttatggaaagagccaaaatttatccaagcattcaacgaaggcaaggacttacacaaacaaacagcaagcataatatatggcattccttatgaagaagtaaataaagaacaaagacaaatagcaaaatcagcaaattttggacttatttatggcatgtcagttgagggatttgctaactattgcataaaaaatggaataccaatggacactcaaacagctcaacacatcgtaaattcattctttaacttctatggtaagatagctgaaaaacataaagaaggaaatcttatcattcaatcacaaggcatagcagaaggttatacttggcttggtagaagatatatagctcaaagacttaacgactaccttaactatcaaatacaaggctctggtgcagaactgcttaaaaaagctgtaatggaaatcaaatccaaatatccttatatcaaaatagtaaatcttgtccatgacgaaattgtagtagaggcttacaaggatgatgcacaagatatagcaaggataatcaagcaagaaatggaaaatgcttgggaatggtgtattcaagaagctcaaaagcttggtgttgatttaacacctgttaagcttgaatgtgaaaaccctacgatatcaaatgtatgggagaagtaa

SEQ ID NO: 4 - protein, 486 aa, Unknown
  Clone 488 from uncultured newly isolated virus
AVGTWITTLQKLGFTYEELEDKEVLDLLSIARLVLPERFKENGFSLDVVLKEVLGIDYKFDKKTIRKTFTPLLMTQEQLEYIASDVIYLPALKEKLDEKFNKRLWLPYILDMEATKILAEVSNNGMPFLKEKAKEELSRLSKELEGLRKELGFNPNSPKETQKVLNTPDTSEATLMKLIISNSSKKAIAEKVIQARKIQKVIAMINKYLNYDRVKGTFWTTTAPSGRMSCDKENLQQIPRSIRYLFGFDENSDKTLVIADYPQIELRLAGVLWKEPKFIQAFNEGKDLHKQTASIIYGIPYEEVNKEQRQIAKSANFGLIYGMSVEGFANYCIKNGIPMDTQTAQHIVNSFFNFYGKIAEKHKEGNLIIQSQGIAEGYTWLGRRYIAQRLNDYLNYQIQGSGAELLKKAVMEIKSKYPYIKIVNLVHDEIVVEAYKDDAQDIARIIKQEMENAWEWCIQEAQKLGVDLTPVKLECENPTISNVWEK

SEQ ID NO: 5 - DNA, 1767 bp, Unknown
  Clone 3173 from uncultured newly isolated virus
atgggagaagatgggctatctttacctaagatgatgaatacaccaaaaccaattcttaaacctcaaccaaaagctttagtagaaccagtgctttgcgatagcattgatgaaataccagcgaaatataatgaaccagtatactttgacttggaaactgacgaagacagaccagttcttgcaagtatttatcaacctcactttgaacgcaaggtgtattgtttaaacctcttgaaagaaaaggtagcaaggtttaaagactggcttcttaaattctcagaaataagaggatggggtcttgactttgacttacgggttcttggctacacctacgaacaacttagaaacaagaagattgtagatgttcagcttgcgataaaagtccagcactacgagagatttaagcagggtgggaccaaaggtgaaggtttcagacttgatgatgtggcacgagatttgcttggtatagaatatccgatgaacaaaacaaaaattcgtgaaaccttcaaaaacaacatgtttcattcatttagcaacgaacaacttctttatgcctcgcttgatgcatacataccacacttgctttacgaacaactaacatcaagcacgcttaatagtcttgtttatcagcttgatcaacaggcacagaaagttgtgatagaaacatcgcaacacggcatgccagtaaaactaaaagcattagaagaagaaatacacagactaactcagctacgcagtgaaatgcaaaagcagataccatttaactataactctccaaaacaaacggcaaaattctttggagtaaatagttcttcaaaagatgtattgatggacttagctctacaaggaaatgaaatggctaaaaaggtgcttgaagcaagacaaatagaaaaatctcttgcttttgcaaaagacctctatgatatagctaaaagaagtggtggtagaatttacggcaacttctttactacaacagcaccatctggcagaatgtcttgctcggatataaatcttcaacagataccgcgtaggcttagatcattcataggctttgatacagaggacaaaaagcttatcaccgcagactttccgcaaattgagcttagacttgcaggtgtgatttggaatgaacctaaattcatagaagcatttaggcaaggtatagaccttcacaagcttacagcatcaatactgtttgataagaacatagaagaagtaagcaaggaagaaaggcaaattggaaaatctgcgaattttgggcttatctatggtattgcaccaaaaggtttcgcagaatattgtatagcgaacggtattaacatgacagaagagcaggcatacgaaatagtcagaaagtggaagaagtattacacaaagattgcagaacaacatcaagtagcatatgaaaggttcaaatacaatgagtatgtagataacgaaacatggcttaacagaacatatcgtgcatggaaaccacaagacctcttgaactatcaaatacaaggcagtggtgcggagctattcaagaaagctatagtattgttaaaagaaacaaagccagacttgaagatagtcaatctcgtgcatgatgagatagtagtagaagcagatagcaaagaagcacaagacttggctaagctaattaaagagaaaatggaggaagcgtgggattggtgtcttgaaaaagcagaagagtttggtaatagagttgctaaaataaaacttgaagtggaggagccacatgtgggtaatacatgggaaaagccttga

SEQ ID NO: 6 - protein, 588 aa, Unknown
  Clone 3173 from uncultured newly isolated virus
MGEDGLSLPKMMNTPKPILKPQPKALVEPVLCDSIDEIPAKYNEPVYFDLETDEDRPVLASIYQPHFERKVYCLNLLKEKVARFKDWLLKFSEIRGWGLDFDLRVLGYTYEQLRNKKIVDVQLAIKVQHYERFKQGGTKGEGFRLDDVARDLLGIEYPMNKTKIRETFKNNMFHSFSNEQLLYASLDAYIPHLLYEQLTSSTLNSLVYQLDQQAQKVVIETSQHGMPVKLKALEEEIHRLTQLRSEMQKQIPFNYNSPKQTAKFFGVNSSSKDVLMDLALQGNEMAKKVLEARQIEKSLAFAKDLYDIAKRSGGRIYGNFFTTTAPSGRMSCSDINLQQIPRRLRSFIGFDTEDKKLITADFPQIELRLAGVIWNEPKFIEAFRQGIDLHKLTASILFDKNIEEVSKEERQIGKSANFGLIYGIAPKGFAEYCIANGINMTEEQAYEIVRKWKKYYTKIAEQHQVAYERFKYNEYVDNETWLNRTYRAWKPQDLLNYQIQGSGAELFKKAIVLLKETKPDLKIVNLVHDEIVVEADSKEAQDLAKLIKEKMEEAWDWCLEKAEEFGNRVAKIKLEVEEPHVGNTWEKP

SEQ ID NO: 7 - DNA, 2250 bp, Unknown
  Clone 4110 from uncultured newly isolated virus
atggggcttgatcaaatacttgatatgagctacttcgttgactcgggggcaacaatgctcaagctcatactcagagggagcggagggaagaatgttgtaacagtgccagcacccttcaacccatacttcttcataaagaagagagacctggatagggctcaaagcatactccccgactacgcaagagtagaggatgctgacgccattactgctgaaggggagcgggttgtgaagataagtgttccaacgccacccctggttagagttgtgagagagaaactccacgaggaaggtatagagtcgtacgaggctgacatcccttacacccggagggtcatgatagacctggatttaaaggtggcgtaccccgagacagtggctgctttcgacatagaggttgacgcaacaaaggggttccccgatatcaacaacccgcagtctagggtcctgtctatctccgtgtacgacgggagcgaggagatattcctatgctcagacgatgagatcgagatgttcaaggagttcaacaagctcctgagaaagtatgatgtgctgataggctggaactcagctgcattcgactacccttacctagttgagagagctaaggtgctcggatactacgtggacgaggagatgttccagcacgtggacatattcgggatattccagacctacttcaagagagagatgagcgacttcaagctcaaaaccgttgccctcaaagtcctgggatccaaggtgccacttggcgccctgctggatttcgagaggcctggagacataaggaagctcacagagttcttcgagaagcgcagggatctcttgaagctatacaacatggatcagactaaggctatatggatgataaacagcgagtcaggtgtgctccaaacatacatcactcaggccaggctcgctaacataataccttggcaccgggtctctccgagaacagatagctcacaggagtacatatcctacaacaatgattgtcgagaccttgtgctgaagaaagctctagctcacaagcccaggatagttttcccatctaagaagaacggtgagaacgaagactgggatgaggatgcaaaggagagcacatacactggagcaatagtcttcaacccgattccagggctatgggagaatgttgtgctcctggacttcgcttcgatgtaccctagggttataatgacgttcaacatctcatacgacacctggacccctaaccctggtgaaaacgatattcttgcgccccacggtggattcatcacctctagagaggggttccttccaacggtgctaagggagcttgaggggtacaggagtctagctaagaagatggttgacgcatatgagccaggtgaccccatgagggtcatatggaacgcaaggcagttcgcattcaaactcatactggtttcagcgtacggtgtagctggattcaggcactctagactctacagggttgagatagctgagagcatcacagggtacacgagagacgcaataatgaaggccagagaggtgatagagaggcacggttggagggtcctctacggggacaccgacagcctgttcttgtacaaccccaagatcacaagcgtggagaaggcttcagaggttgcatcaagcgagctgctcccagccataaactcctttataagagactacgtggtggagagatggagggtcccgaggagcagggttgtgttggagttcaaggttgacagggtgtactcgaagctgaagctgctgagtgtgaagaagaggtactatggcttggttgcgtgggaggagaggatgctcgagcaaccctacattcagatcaagggcctggaagcaaggagaggtgattggcctgacctggtcaaggagatacagtcagaggtgatcaagctgtacctcctagagggacccatagctgtagacaggtatctgaaggagatgaagaggaagctcctgtccggggagatacccctggagaagctggttatcaagaagcatctgaacaagaggcttgacgagtataagcataacgcgccccactacagggctgcaaggaagctcctagagatgaggttccccgttagaaccggggatagaatagagttcatctaccttgacgacaaggtgatccccatggttccagggctgaagctatcagaggttgacctgaagaagtggtggaggaaatacgttgtcccggtagtcgagagactggagatagagagcagagggagctag

SEQ ID NO: 8 - protein, 749 aa, Unknown
  Clone 4110 from uncultured newly isolated virus
MGLDQILDMSYFVDSGATMLKLILRGSGGKNVVTVPAPFNPYFFIKKRDLDRAQSILPDYARVEDADAITAEGERVVKISVPTPPLVRVVREKLHEEGIESYEADIPYTRRVMIDLDLKVAYPETVAAFDIEVDATKGFPDINNPQSRVLSISVYDGSEEIFLCSDDEIEMFKEFNKLLRKYDVLIGWNSAAFDYPYLVERAKVLGYYVDEEMFQHVDIFGIFQTYFKREMSDFKLKTVALKVLGSKVPLGALLDFERPGDIRKLTEFFEKRRDLLKLYNMDQTKAIWMINSESGVLQTYITQARLANIIPWHRVSPRTDSSQEYISYNNDCRDLVLKKALAHKPRIVFPSKKNGENEDWDEDAKESTYTGAIVFNPIPGLWENVVLLDFASMYPRVIMTFNISYDTWTPNPGENDILAPHGGFITSREGFLPTVLRELEGYRSLAKKMVDAYEPGDPMRVIWNARQFAFKLILVSAYGVAGFRHSRLYRVEIAESITGYTRDAIMKAREVIERHGWRVLYGDTDSLFLYNPKITSVEKASEVASSELLPAINSFIRDYVVERWRVPRSRVVLEFKVDRVYSKLKLLSVKKRYYGLVAWEERMLEQPYIQIKGLEARRGDWPDLVKEIQSEVIKLYLLEGPIAVDRYLKEMKRKLLSGEIPLEKLVIKKHLNKRLDEYKHNAPHYRAARKLLEMRFPVRTGDRIEFIYLDDKVIPMVPGLKLSEVDLKKWWRKYVVPVVERLEIESRGS

SEQ ID NO: 9 - DNA, 1992 bp, Unknown
  Clone 2323 from uncultured newly isolated virus
atgatagacctggatttaaaagtagcgtacccagagactgtagctgctttcgacatagaggttgacgcaacaaaggggttccccgatatcaacaacccccagtctagagtcctgtctatctcagtgtacgatgggagcgaagagatattcctatgctcagacgatgaggtcgagatgttcaaggagttcaacaggctcctgaggaagtatgatgtgatgatagggtggaactcagctgcattcgactacccttacctcgtagagagagctaagatgctcggatactacgtagacgaggagatgttccagcacgtggacatattcgggatattccagacctacttcaagagggagatgagcgacttcaagctcaaaacagttgccctcaaggtcctcggatccaaggtgccacttggcggccctgttggatttcgagaggccaggggacatagctaagctcacggagttctttgagaggcgcagggatctcttgagactctacaacatggatcagaccaggcgatatggatgataaacagcgagtcaggcgtgctccagacctacatcacacaggctaggctcaccaacataataacctggcacagggacctctctgagaagcagatagctcacaggaagtatatatcctacaacaggatggtcgagaaccttgtcttgaagaaagctctagctcacaagccgaggatagtgttcccatccaagaagaacggcgagaacaacgagtgggatgaagacaataaagagagctcatacacaggagctatagtcttcaaccccgtgccagggctatgggagaacgttgtcctcctggacttcgcaaccatgtaccctagggtcataatgacattcaacatctcatacgacacctggaccccgaaccccggtgagagcgatattcttgcgccccacggtggattcatcacctctagagaggggttccttccaacagtgctaagggagcttgaggggtacaggagtctagctaagaagatggttgacgcatatgagccaggtgaccccatgagagttatatggaatgcaagacagttcgcgttcaaactcatactggtttcagcgtacggtgtagctggattcaggcactctaggctctacagggttgagatagccgagagcatcactgggtacaccagagacgcaataatgaaggcgagagaggtgatagagagtcacggttggagggtcctctacggtgacactgacagcctgttcttgtacaaccccggggtctcgagcgctgagaaggctgcagaggttgcatcaagcgagctacttccagccataaactcctttataagagactacgctgtggagagatggagggttccgaggagcagggttgtgttggagttcaaggatgacagggtgtactcaaagctgaagctcctgagtgtgaagaagaggtactatggcttggtatcgtgggaggagaggatgctcgagaaaccctacattcagatcaagggccttgaggctaggaggggtgattggcctgacctggtcaaggagatacagtcagaggtgatcaagctgtacctcctagagggcccaagagctgttgactcgtatctcaaggagatgaagaggaagctcctatcgggggagatacccttggagaagctggttatcaagaagcacctgaacaagaggctgggcgagataagcataatgcgccccactaccagggctgccaggaagctcctagagatgaggttccccgttagaacaggggatagaatagagttcatctaccttgacgacaaggtgatccccatggttccagggctgaagctttcagaggttgacctgaggaagtggtggaggaaatacgttgtcccagtagtggagagactggagatagagagcagagggagcttgctagacaggatgcggccgcttgtatctgatacgacattcaggatccgaattcgtcgacgatatcttcccctatag

SEQ ID NO: 10 - protein, 661 aa, Unknown
  Clone 2323 from uncultured newly isolated virus
MIDLDLKVAYPETVAAFDIEVDATKGFPDINNPQSRVLSISVYDGSEEIFLCSDDEVEMFKEFNRLLRKYDVMIGWNSAAFDYPYLVERAKMLGYYVDEEMFQHVDIFGIFQTYFKREMSDFKLKTVALKVLGSKVPLGGPVGFREARGHSAHGVLEAQGSLETLQHGSDQAIWMINSESGVLQTYITQARLTNIITWHRDLSEKQIAHRKYISYNRMVENLVLKKALAHKPRIVFPSKKNGENNEWDEDNKESSYTGAIVFNPVPGLWENVVLLDFATMYPRVIMTFNISYDTWTPNPGESDILAPHGGFITSREGFLPTVLRELEGYRSLAKKMVDAYEPGDPMRVIWNARQFAFKLILVSAYGVAGFRHSRLYRVEIAESITGYTRDAIMKAREVIESHGWRVLYGDTDSLFLYNPGVSSAEKAAEVASSELLPAINSFIRDYAVERWRVPRSRVVLEFKDDRVYSKLKLLSVKKRYYGLVSWEERMLEKPYIQIKGLEARRGDWPDLVKEIQSEVIKLYLLEGPRAVDSYLKEMKRKLLSGEIPLEKLVIKKHLNKRLGEISIMRPTTRAARKLLEMRFPVRTGDRIEFIYLDDKVIPMVPGLKLSEVDLRKWWRKYVVPVVERLEIESRGSLLDRMRPLVSDTTFRIRIRRRYLPL

SEQ ID NO: 11 - DNA, 591 bp, Unknown
  Clone 653 from uncultured newly isolated virus
atgcactggtctctcttagatgagtaccttaactctggagcgataaggatgagcgaggggtccatggagtcagtcgcatacatagaggttgcaaagaagatactctactgcagaaagtgcggtttcaatgtgaagcacccataccccggatccggctcgttggatgcaaagataatgatagttggggagagcccctcaccccacaggaagtcatttgagaacttctcggagaggagcagggaggttgttgatgctgttctatctgcactgggtctatccagggagacagtgtacatgactaacgctgtgaagtgtcctctctaccatctggagatggaggacaggatgaagtacattgacttatgcttcgagcacctgctaagcgagatacagattgtgaaacctaagatcgttatcagcttcggtgtcatagctgagagagctgtttccaaggcattgagggttagcacacataagttcttccatgtagctctaccccatccgatgaaagtggtgtatggccagatgacgctggaagactaccttagggaggtgaagaggagatggggcttgatcaaatacttgatataa

SEQ ID NO: 12 - protein, 196 aa, Unknown
  Clone 653 from uncultured newly isolated virus
MHWSLLDEYLNSGAIRMSEGSMESVAYIEVAKKILYCRKCGFNVKHPYPGSGSLDAKIMIVGESPSPHRKSFENFSERSREVVDAVLSALGLSRETVYMTNAVKCPLYHLEMEDRMKYIDLCFEHLLSEIQIVKPKIVISFGVIAERAVSKALRVSTHKFFHVALPHPMKVVYGQMTLEDYLREVKRRWGLIKYLI

SEQ ID NO: 13 - DNA, 1029 bp, Unknown
  Clone 967 from uncultured newly isolated virus
atgcaaaaagaaataccatttaactacaattcacctaaacaaacagcaaagctttttggtatagatagttcttcaaaagatgtgcttatggatttagcattaaggggtaatgaggtagctaagaaagttcttgaagcaagacaaatagaaaagtctttagcttttgcaaaagacctttatgatatagctaaaaagaatggtggtagaattcacggaaacttctttactactaccgcaccatcgggtagaatgtcttgttcagatataaacttacaacaaatacctcgcaggttaagacaattcataggttttgaaacagaagataaaaaacttataactgctgactttcctcaaatagaacttaggcttgcgggtgtaatgtggaatgaaccagaatttttaaaagcgtttagggatggtatagacttacataaactaacagcttcaatcctgtttgataaaaaaattaatgaggtaagtaaagaagaaagacaaataggcaaatcagcaaactttggtttaatttacggtatctctccaaagggttttgctgaatattgtataagcaacggaataaatataacagaagaaatggctattgagattgtaaagaaatggaagaagttttacagaaaaatagcagaacaacaccaactggcttacgaaaggttcaagtatgctgaatttgtagataatgaaacatggttgaatagaccttacagggcttataaacctcaggaccttctcaattatcaaattcaaggaagcggtgctgagttgtttaaaaaagctataattctacttaaagaaacaaaaccagaccttaagcttgtaaatcttgtgcatgatgagattgtagtggaaacctcaacagaagaagctgaagatatagctttgttggtaaaacaaaagatggaagaggcttgggattattgtttagaaaaggctaaggaatttggtaataatgtggcggatataaaacttgaagtagaaaaacctaacataagcagtgtatgggaaaaggagtaa

SEQ ID NO: 14 - protein, 342 aa, Unknown
  Clone 967 from uncultured newly isolated virus
MQKEIPFNYNSPKQTAKLFGIDSSSKDVLMDLALRGNEVAKKVLEARQIEKSLAFAKDLYDIAKKNGGRIHGNFFTTTAPSGRMSCSDINLQQIPRRLRQFIGFETEDKKLITADFPQIELRLAGVMWNEPEFLKAFRDGIDLHKLTASILFDKKINEVSKEERQIGKSANFGLIYGISPKGFAEYCISNGINITEEMAIEIVKKWKKFYRKIAEQHQLAYERFKYAEFVDNETWLNRPYRAYKPQDLLNYQIQGSGAELFKKAIILLKETKPDLKLVNLVHDEIVVETSTEEAEDIALLVKQKMEEAWDYCLEKAKEFGNNVADIKLEVEKPNISSVWEKE

SEQ ID NO: 15 - DNA, 2253 bp, Unknown
  Clone 2783 from uncultured newly isolated virus
atgagctacttcgttgactcaggggcaacaatgctcaagctcatactcagggggagcggaggtaagaaggttgtaacagtgccagcccccttcaacccatactttttcataaagaagagagacctggatagggctcaaagcatactcccagtacttacgcttagcgtggaggatgctgacgccattacagctgaaggggagagggttgtgaagataagtgttccaacgccacccctggtcagggttgtgagggagaaactccacgaggaggggatagagtcgtacgaggctgatatcccttacaccaggagggtcatgatagacctggatttaaaggttgcgtaccctgagaccgttgcagctttcgacatagaggttgacgcaacaagggggttccccgatatcaacaacccgcagtcaagggttctctctatctccgtgtacgacgggagcgaggagatattcctatgctcagacgatgagatcgagatgttcaaggagttcaacaagctcctgaggaggtacgatgtgctgataggctggaactcagctgcattcgactacccttacctagtagagagagcaaaggtgctcggatactacgttgacgaggagatgttccagcacgtggacatattcgggatattccagacctacttcaagagggagatgagcgacttcaagctcaagactgtagccctcaaggtcctgggatccaaggtgccacttggcgccctgctggatttcgagaggcctggtgacataaggaagctcacggagttcttcgagaggcgcagggatctccttagactctacaacatggatcagacacaggcgatatggatgataaacagcgagtcaggtgtgctccagacctacatcacccaggctaggctcgctaacataataccttggcaccgggatctctccgagaagcagattgctcacaggaagtatatatcctacaacaagatcgtcgagaaccttgtcttgaagaaagctctatctcacagtccaaggatagttttcccatctaagaagaacggtgagaacgaagactgggatgaggatgcaaaggagagcacatacactggagcaatagtgttcaacccgattccagggctatgggagaatgttgtgctcctggacttcgcttcgatgtaccctagggttataatgacgttcaacatctcatacgacacatggacccctagccccggtgaaaacgacattcttgcgccccacggtggattcatcacctccagggaggggttccttccaacggtgctaagggagcttgaggggtacaggagtctagctaagaagatggttgacgcatatgagccaggcgaccccatgagggtcatatggaacgccaggcagttcgcgttcaaactcatactggtttcggcttacggtgtagcaggattcaggcactctagactctacagggttgagatagccgagagcatcacggggtacaccagggacgccataatgaaggcgagagaggtgatagagaggcacggttggagggtcctctacggggacaccgacagcctgttcttgtacaaccccaagatctcaagcgtggagaaggctgctgaggttgcatcaagcgagcttctcccagccataaactcctttataagagactacgtggtggagagatggagggttccgaggagcagggttgtgttggagttcaaggttgacagggtgtactcgaagctgaagctgctgagtgtgaagaagaggtactacggcttggttgcgtgggaggagaggatgcttgagcaaccctacattcagatcaagggccttgaggctaggaggggtgattggcctgacctggtgaaggagatacagtcagaggtgatcaagctgtacctcctggagggacccatggctgtagacaggtatctcagggagatgaagaggaagctcctgtccggggagatacccttggagaagcttgttatcaagaagcatctgaacaagaggcttgacgagtataagcataacgcgccccactacagggctgcaaagaagctcctggagatgaggttcccggttagaactggggatagaatagagttcatataccttgacgacaaggtgatccccatggttccaggactgaagctatcagaggttgacctgaagaagtggtggaggaaatacgttgtcccggtggtcgagagactggagatagagagcagagggagcttgctggacaggtacctagggtga

SEQ ID NO: 16 - protein, 750 aa, Unknown
  Clone 2783 from uncultured newly isolated virus
MSYFVDSGATMLKLILRGSGGKKVVTVPAPFNPYFFIKKRDLDRAQSILPVLTLSVEDADAITAEGERVVKISVPTPPLVRVVREKLHEEGIESYEADIPYTRRVMIDLDLKVAYPETVAAFDIEVDATRGFPDINNPQSRVLSISVYDGSEEIFLCSDDEIEMFKEFNKLLRRYDVLIGWNSAAFDYPYLVERAKVLGYYVDEEMFQHVDIFGIFQTYFKREMSDFKLKTVALKVLGSKVPLGALLDFERPGDIRKLTEFFERRRDLLRLYNMDQTQAIWMINSESGVLQTYITQARLANIIPWHRDLSEKQIAHRKYISYNKIVENLVLKKALSHSPRIVFPSKKNGENEDWDEDAKESTYTGAIVFNPIPGLWENVVLLDFASMYPRVIMTFNISYDTWTPSPGENDILAPHGGFITSREGFLPTVLRELEGYRSLAKKMVDAYEPGDPMRVIWNARQFAFKLILVSAYGVAGFRHSRLYRVEIAESITGYTRDAIMKAREVIERHGWRVLYGDTDSLFLYNPKISSVEKAAEVASSELLPAINSFIRDYVVERWRVPRSRVVLEFKVDRVYSKLKLLSVKKRYYGLVAWEERMLEQPYIQIKGLEARRGDWPDLVKEIQSEVIKLYLLEGPMAVDRYLREMKRKLLSGEIPLEKLVIKKHLNKRLDEYKHNAPHYRAAKKLLEMRFPVRTGDRIEFIYLDDKVIPMVPGLKLSEVDLKKWWRKYVVPVVERLEIESRGSLLDRYLG

SEQ ID NO: 17 - DNA, 333 bp, Unknown
  Clone 2072 from uncultured newly isolated virus
atgctcgtgctaagcactacggagaagctagtcctgttagctgtcgtggttgagacagagtatggcaagaagccaaccaccaaggggaaggtgtacagtaggtatacagagctatcaaggttagctggagtggagcccgtgacaccaaggagaaccctcgatgtattgaagaacctggctgagaaggggatcctgtgggtcaaggttgacagcttcggaaggtatggtaggacgacggttgtcaaactactagcacccccaaccaccctatgccaggagctagccgaagatttgttgataggcgaggtggcggaggaggtctgcagggggtga

SEQ ID NO: 18 - protein, 110 aa, Unknown
  Clone 2072 from uncultured newly isolated virus
MLVLSTTEKLVLLAVVVETEYGKKPTTKGKVYSRYTELSRLAGVEPVTPRRTLDVLKNLAEKGILWVKVDSFGRYGRTTVVKLLAPPTTLCQELAEDLLIGEVAEEVCRG

SEQ ID NO: 19 - DNA, 294 bp, Unknown
  Clone 2123 from uncultured newly isolated virus
atgggagcgtgccctccacttactggtaaggtctacgcgagatacgctgagctcgcgaggctccacaaggtgaaacccatcaccatgaggaggttgcaggacgtcctgaagggcctagcgaaggccggaatactgagggttgtggttcgcagcttcggcaggtacggtaagacgtcgatcatagtgttgaggcaaccaccgcaaaccctgtgcccaatactcacagaggatctagtggtaggggagatggcggaggagatctgcagagatacccagcccataccccccgggtga

SEQ ID NO: 20 - protein, 97 aa, Unknown
  Clone 2123 from uncultured newly isolated virus
MGACPPLTGKVYARYAELARLHKVKPITMRRLQDVLKGLAKAGILRVVVRSFGRYGKTSIIVLRQPPQTLCPILTEDLVVGEMAEEICRDTQPIPPG

SEQ ID NO: 21 - DNA, 29 bp, Artificial Sequence
  Synthetic oligonucleotide
gagcagtatcagatacaagcggccgcatc

SEQ ID NO: 22 - DNA, 28 bp, Artificial Sequence
  Synthetic oligonucleotide
tcgtcatagtctatgttcgccggcgtag

SEQ ID NO: 23 - DNA, 37 bp, Artificial Sequence
  Synthetic oligonucleotide
tgtctcagacagtcagactgctgacagatgacttgca

SEQ ID NO: 24 - DNA, 41 bp, Artificial Sequence
  Synthetic oligonucleotide
aacgtgcaagtcatctgtcagcagtctgactgtctgagaca

SEQ ID NO: 25 - protein, 588 aa, Artificial Sequence
  D49A mutant of Clone 3173
MGEDGLSLPKMMNTPKPILKPQPKALVEPVLCDSIDEIPAKYNEPVYFALETDEDRPVLASIYQPHFERKVYCLNLLKEKVARFKDWLLKFSEIRGWGLDFDLRVLGYTYEQLRNKKIVDVQLAIKVQHYERFKQGGTKGEGFRLDDVARDLLGIEYPMNKTKIRETFKNNMFHSFSNEQLLYASLDAYIPHLLYEQLTSSTLNSLVYQLDQQAQKVVIETSQHGMPVKLKALEEEIHRLTQLRSEMQKQIPFNYNSPKQTAKFFGVNSSSKDVLMDLALQGNEMAKKVLEARQIEKSLAFAKDLYDIAKRSGGRIYGNFFTTTAPSGRMSCSDINLQQIPRRLRSFIGFDTEDKKLITADFPQIELRLAGVIWNEPKFIEAFRQGIDLHKLTASILFDKNIEEVSKEERQIGKSANFGLIYGIAPKGFAEYCIANGINMTEEQAYEIVRKWKKYYTKIAEQHQVAYERFKYNEYVDNETWLNRTYRAWKPQDLLNYQIQGSGAELFKKAIVLLKETKPDLKIVNLVHDEIVVEADSKEAQDLAKLIKEKMEEAWDWCLEKAEEFGNRVAKIKLEVEEPHVGNTWEKP

SEQ ID NO: 26 - protein, 588 aa, Artificial Sequence
  E51A/F418Y mutant of Clone 3173
MGEDGLSLPKMMNTPKPILKPQPKALVEPVLCDSIDEIPAKYNEPVYFDLATDEDRPVLASIYQPHFERKVYCLNLLKEKVARFKDWLLKFSEIRGWGLDFDLRVLGYTYEQLRNKKIVDVQLAIKVQHYERFKQGGTKGEGFRLDDVARDLLGIEYPMNKTKIRETFKNNMFHSFSNEQLLYASLDAYIPHLLYEQLTSSTLNSLVYQLDQQAQKVVIETSQHGMPVKLKALEEEIHRLTQLRSEMQKQIPFNYNSPKQTAKFFGVNSSSKDVLMDLALQGNEMAKKVLEARQIEKSLAFAKDLYDIAKRSGGRIYGNFFTTTAPSGRMSCSDINLQQIPRRLRSFIGFDTEDKKLITADFPQIELRLAGVIWNEPKFIEAFRQGIDLHKLTASILFDKNIEEVSKEERQIGKSANYGLIYGIAPKGFAEYCIANGINMTEEQAYEIVRKWKKYYTKIAEQHQVAYERFKYNEYVDNETWLNRTYRAWKPQDLLNYQIQGSGAELFKKAIVLLKETKPDLKIVNLVHDEIVVEADSKEAQDLAKLIKEKMEEAWDWCLEKAEEFGNRVAKIKLEVEEPHVGNTWEKP

SEQ ID NO: 27 - protein, 588 aa, Artificial Sequence
  D49A/F418Y mutant of Clone 3173
MGEDGLSLPKMMNTPKPILKPQPKALVEPVLCDSIDEIPAKYNEPVYFALETDEDRPVLASIYQPHFERKVYCLNLLKEKVARFKDWLLKFSEIRGWGLDFDLRVLGYTYEQLRNKKIVDVQLAIKVQHYERFKQGGTKGEGFRLDDVARDLLGIEYPMNKTKIRETFKNNMFHSFSNEQLLYASLDAYIPHLLYEQLTSSTLNSLVYQLDQQAQKVVIETSQHGMPVKLKALEEEIHRLTQLRSEMQKQIPFNYNSPKQTAKFFGVNSSSKDVLMDLALQGNEMAKKVLEARQIEKSLAFAKDLYDIAKRSGGRIYGNFFTTTAPSGRMSCSDINLQQIPRRLRSFIGFDTEDKKLITADFPQIELRLAGVIWNEPKFIEAFRQGIDLHKLTASILFDKNIEEVSKEERQIGKSANYGLIYGIAPKGFAEYCIANGINMTEEQAYEIVRKWKKYYTKIAEQHQVAYERFKYNEYVDNETWLNRTYRAWKPQDLLNYQIQGSGAELFKKAIVLLKETKPDLKIVNLVHDEIVVEADSKEAQDLAKLIKEKMEEAWDWCLEKAEEFGNRVAKIKLEVEEPHVGNTWEKP

SEQ ID NO: 28 - DNA, 43 bp, Artificial Sequence
  Synthetic oligonucleotide
gtctgaggccctcagtccagttacgctggagtctgaggctcgt

SEQ ID NO: 29 - DNA, 44 bp, Artificial Sequence
  Synthetic oligonucleotide
ctgtgagggccttcattagaaaaactcatcgagcatcaagtgaa

SEQ ID NO: 30 - DNA, 1176 bp, Unknown
  Clone 347 of uncultured newly isolated virus
atgaggcgggctgagaaagaggcgggcaggctagcgggtgctgtgaggcgtattgagtctctaacgagggagcttgtagcccagttcaggaaggctatatttgctggtgggcgtatagcggagggcagacctgttctgggcacagctgtcaagagggctacgtttttgggggttcacgcattcatgttccatgacgtcaggctactcgaccgggcactcgaatctagcccctctgaacacgagaaagagctctcggatttgcttgggttcaagcgtgtggaggacatgagagcgtgtttgagatacgtatttgaaacgctgggggtaaagcaggtattgagcttcgagtcaagcaacctagactctactagggagtgtgaggattaccgcaggcttgggattgggttgagccacctcgggctcatattaagggagaacccggatatcccaggctcaccgagggcggtatccataaaccccctcgtgccctggattytaagggctatagttaacttcgcccggggagacataggagctggctacattgcctcggtggaagcctccataggcaggggcaagacaacaacaatatattacacaatacgctctgtcctaaacttgcttggtcacccagaccctgatggtgccacacaaaggctaattatacttgacccagtagagttcattgaagtgtctgaatctcttgcggagaggcgtgagaaggtagcattatctgtcgtggataatgctagtataattctaccgaagcaatgggcttcatatggaggggagctcagaaagttcttcttgagggctaacactatggtatcggtcattagggggctgtccgccgtgaacatattcgtggctaacgcccctggggagcttgcctccttcgtgaggaacgccacaatagtgaggtttgagggagaggcactcgatcaagactcactccttgttacagcctatgtattgagaagggcgggcatcagggttacagccagcacgggggaggagaagctcataaagaaggagaggctagccaccgtgtatgtgtacccgttcctcaagctcccggagccgttatactacaaagacatgacagcaaagctagaaacagtccggaaagagctcaaagaggcggagaagatagttaaacagtactggagaaggtga

SEQ ID NO: 31 - protein, 391 aa, Unknown
  Clone 347 of uncultured newly isolated virus
MRRAEKEAGRLAGAVRRIESLTRELVAQFRKAIFAGGRIAEGRPVLGTAVKRATFLGVHAFMFHDVRLLDRALESSPSEHEKELSDLLGFKRVEDMRACLRYVFETLGVKQVLSFESSNLDSTRECEDYRRLGIGLSHLGLILRENPDIPGSPRAVSINPLVPWIXRAIVNFARGDIGAGYIASVEASIGRGKTTTIYYTIRSVLNLLGHPDPDGATQRLIILDPVEFIEVSESLAERREKVALSVVDNASIILPKQWASYGGELRKFFLRANTMVSVIRGLSAVNIFVANAPGELASFVRNATIVRFEGEALDQDSLLVTAYVLRRAGIRVTASTGEEKLIKKERLATVYVYPFLKLPEPLYYKDMTAKLETVRKELKEAEKIVKQYWRR

SEQ ID NO: 32 - DNA, 2110 bp, Unknown
  Clone 74 of uncultured newly isolated virus
gctctgtgcttgttagcagaattgatgatgcagaattgataaacaacgacgtggagagattgttagaaaatgtaaataacatgcctgacaaagatagagacctttgtctctatgcttatatcaataccgcaaaacgtgttttgagtgagaaagtgtttaactctgtttataccgtattaaaaggcaaaggtgtagatgtagaaaagtatttgaatatgagctttgcagatgatgacgatgacgcagaaccgccaccaattggcggtaataataccgaaccaccgaataacaatggtggtgctgataacgaaccgcaagaagataatcgttttgctgaaattaaagaagtcaaacaactaaccgcgcctctgcctgatgtagagcttataacagactttaagcaaattaaagaccacatacaatacgatggaactatatacatagacgtagaagcagatgttgaaacgcaacaacctattttgctcgctctctatcaaaaacactggaaaaaggtttatgcggtagatttacgtaaagtcaagctggagcaagtcaaagaatggcttctcagatttaacgtaataagtggctgggggcttaactatgaccttgtcaggcttggtttttcttatgaagagttaaaagaccatgtcgtgttagacttgcttttacttgcacgggaaaagctttacacaagagacagctttaagttagatgatgtgttaaaagatgtgcttggagtggagtatccatttgacaaaacgaaaatcagaaaaacattcaaaaacacactttactttacacaagaacagctacaatatgcaggtctggatgtttattatttgcctaaactgtttgatgccatttctgatgatagtttaagcatagttcaacagttagaccaagaagctttaaaagtttgtgtggatacaagccaaagaggaatgccgttcttggttgaagaagcgaaagcaaagctaattgttctacgacaagaacttgatgttataaataaagaacttggctttaatccacgctccccgcaacaaactaaaaacgctttatcggttcaagatacgagggaagagacgcttcaagacctaataatcaacaatggagtaaggaaggaaatagcagagaaggttttacttgcaagaaagatagctaaagaaatttctatgcttgagacgtatatacagcatggagttagagtgaaaggcatcttttggacaacacaggctccaagcggacgtatgagttgcaatgatgaaaatcttcagcaggttccaagaagtttgagagacctgtttggctttactgaagataacgataaagtccttataacagctgactttccgcagatagaattgagactggcaggtgctttatggcgtgagccgaaatttgtagaagcgtttagaaaaggtgaagaccttcataaaataacggcttcaattatctatggcgtgcctgtagatgaagtatctaaggagcaaaggcaggtggcaaagtctgccaattttggactaatttacggagcttctccacaaggtttccaaagatattgcataagcaatggaattccgatggatttagaaacggcacaactaattcacacgaaattctttgagacttatacaaagatagctaaagaacatgagcttgtcaaagattactttagatacaacacagaagctgaaggtgaaacttggcttggtagaaaatacgtagctaaatctccacagcaaatgcttaactatcaaatacaaggttcaggtgcagagttgtttaagaagacgatagtagagcttaagaagaagtatccaagccttgctattgttaatttggtgcatgacgagatagtgattgaagcagacagacaaacagcagaggatatagctttaattgtaaaagcggagatggaacaagcttgggagtggtgtttagaagaagctaaacagcaaggtaggttgattgaagaatttaaacttgaagttgaaatgcctaacattagcaaaaaatgggaaaaaccataaaggaggtggcaccatgataaaagtctggttggaccattttcttttgtatgttgctgacccagacaaactccaga

SEQ ID NO: 33 - protein, 677 aa, Unknown
  Clone 74 of uncultured newly isolated virus
SVLVSRIDDAELINNDVERLLENVNNMPDKDRDLCLYAYINTAKRVLSEKVFNSVYTVLKGKGVDVEKYLNMSFADDDDDAEPPPIGGNNTEPPNNNGGADNEPQEDNRFAEIKEVKQLTAPLPDVELITDFKQIKDHIQYDGTIYIDVEADVETQQPILLALYQKHWKKVYAVDLRKVKLEQVKEWLLRFNVISGWGLNYDLVRLGFSYEELKDHVVLDLLLLAREKLYTRDSFKLDDVLKDVLGVEYPFDKTKIRKTFKNTLYFTQEQLQYAGLDVYYLPKLFDAISDDSLSIVQQLDQEALKVCVDTSQRGMPFLVEEAKAKLIVLRQELDVINKELGFNPRSPQQTKNALSVQDTREETLQDLIINNGVRKEIAEKVLLARKIAKEISMLETYIQHGVRVKGIFWTTQAPSGRMSCNDENLQQVPRSLRDLFGFTEDNDKVLITADFPQIELRLAGALWREPKFVEAFRKGEDLHKITASIIYGVPVDEVSKEQRQVAKSANFGLIYGASPQGFQRYCISNGIPMDLETAQLIHTKFFETYTKIAKEHELVKDYFRYNTEAEGETWLGRKYVAKSPQQMLNYQIQGSGAELFKKTIVELKKKYPSLAIVNLVHDEIVIEADRQTAEDIALIVKAEMEQAWEWCLEEAKQQGRLIEEFKLEVEMPNISKKWEKP

SEQ ID NO: 34 - DNA, 1931 bp, Unknown
  Clone 2783-GBS from uncultured newly isolated virus
acaatacgatggaactatatacatagacgtagaagcagatgttgaaacgcaacaacctattttgctcgctctctatcaaaaacactggaaaaaggtttatgcggtagatttacgtaaagtcaagctggagcaagtcaaagaatggcttctcagatttaacgtaataagtggctgggggcttaactatgaccttgtcaggcttggtttctcttatgaagagttaaaagaccatgtcgtgttagacttgcttttacttgcacgggaaaagctttacaaaagagacagctttaagttagatgatgtgttaaaagatgtgcttggagtggagtatccatttgacaaaacgaaaatcagaaaaacattcaaaaacacactttactttacacaagaacagctacaatatgttggtcttgatgtttattatttgcctaaactgtttgatgccatttctgatgacagtttaagcatagttcaacagttagaccaagaagctttaaaggtttgtgtggatacaagccaaagaggaatgccgttcttggttgaagaagcgagagcaaagctaattgttctacgacaagaacttgatgttataaccaaagagcttggctttaatccacgctccccgcaacaaactaaaaacgctttatcggttcaagatacgagggaagagacgcttcaagacctaataatcaacaatggagtaagaaaggaaatagcggagaaggttttacttgcaagaaagatagctaaagaaatttctatgcttgagacgtatatacagcacggagttagagtgaaaggcatcttttggacaacacaggctccaagcggacgtatgagttgcaatgatgaaaatcttcagcaggttccaagaagtttgagagacttgtttggctttactgaagataacgataaagtccttataacagctgactttccgcagatagaattgagactggcaggtgctttatggcgtgagccgaaatttgtagaagcgtttagaaaaggtgaagaccttcataaaataacggcttcaattatttatggcgtgcctgtagatgaagtatctaaggagcaacggccaggtggcaaagtctgccaattttggactaatttacggagcttctccacaaggtttccaaagatattgcataagcaatggaattccgatggatttagagacggcacaactaattcacacgaaattctttgagacttatacaaagatagctaaagaacatgagcttgtcaaagattactttagatacaacacagaagctgaaggtgaaacttggcttggtagaaaatacgtagctaaatctccacagcaaatgcttaactatcaaatacaaggttcaggtgcagagttgtttaagaagacgatagtagagcttaagaagaagtatccaagccttgctattgttaatttggtgcatgacgagatagtgattgaagcagacagacaaacagcagaggatatagctttaattgtaaaagcggagatggaacaagcttgggagtggtgtttagaagaagctaaacagcaaggtaggttgattgaagaatttaaacttgaagttgaaatgcctaacattagcaaaaaatgggaaaaaccataaaggaggtggcaccatgataaaagtctggttggaccattttcttttgtatgttgctgacccagacaaacttccagccattttggacgaagccgatttcagtaaagctttaataccacgaaagatttggaagactgtttatcccattatgcagaaaaagggctttgctatagcaagagtagtgttaatagaggaggagccttacgatgaatggacttgtgagacatgaatcgtgtaagatgtaataagcttatcagatgcaaatggttagacgagcaaaaagaactcattattacgctttacgaaattggcttt

SEQ ID NO: 35 - protein, 538 aa, Unknown
  Clone 2783-GBS from uncultured newly isolated virus
QYDGTIYIDVEADVETQQPILLALYQKHWKKVYAVDLRKVKLEQVKEWLLRFNVISGWGLNYDLVRLGFSYEELKDHVVLDLLLLAREKLYKRDSFKLDDVLKDVLGVEYPFDKTKIRKTFKNTLYFTQEQLQYVGLDVYYLPKLFDAISDDSLSIVQQLDQEALKVCVDTSQRGMPFLVEEARAKLIVLRQELDVITKELGFNPRSPQQTKNALSVQDTREETLQDLIINNGVRKEIAEKVLLARKIAKEISMLETYIQHGVRVKGIFWTTQAPSGRMSCNDENLQQVPRSLRDLFGFTEDNDKVLITADFPQIELRLAGALWREPKFVEAFRKGEDLHKITASIIYGVPVDEVSKEQRQVAKSANFGLIYGASPQGFQRYCISNGIPMDLETAQLIHTKFFETYTKIAKEHELVKDYFRYNTEAEGETWLGRKYVAKSPQQMLNYQIQGSGAELFKKTIVELKKKYPSLAIVNLVHDEIVIEADRQTAEDIALIVKAEMEQAWEWCLEEAKQQGRLIEEFKLEVEMPNISKKWEKP

SEQ ID NO: 36 - DNA, 3023 bp, Unknown
  Clone 1160 from uncultured newly isolated virus
tggaaaggatagacaaacgcatcattgaggaccaagcacgtaagtttgcaaatattaagttgcttcaggacacactgataagagaaggttataaagaccaaaaggtaaagcttctttctgctattgacaacttactttcaagaatacttttcgctcttagaattttcactgatgttcttcaaattccgcaagaagagtgggaagagcttttgacacaaatagctttatacgtggatacaagcttatcaacattctataaactattcctgccacgtgagaaaaggcttgaagaagagcttgtagattttctgacacaattgactgatattctttacaagactatcaacgatagaaacagaccagacttgcctcggactttgggaggagcttctttggataagcttatcaaaatagcaaaggtgcaaataccaagctcgcaagtcttgaagtattctaaagatgttcttctcaagaaatacaaaacagcaagagcttatttattcgcaagctctgtgcttgttagcagaattgatgatgcagaattgataaacaacgacgtggagagattgttagaaaatgtaaataacatgcctgacaaagatagagacctttgcctttatgcttatatcaatactgcaaaacgtgttttgagtgagaaagtgtttaactctgtttataccgtattgaaaagcaaaggtgtagatgtggataagtatttgaatatgagctttgtagatgacgacgatgacacagaaccaccaccaatatcaccgaataacaatggtggtgctgataacgaaccgcaagaagataatcgttttgctgaaattaaagaagtcaagcaactaaccgcacctctgcctgatgtagagcttataacagactttaagcaaattaaagaccacatacaatacgatggaactatatacatagacgtagaagcagatgttgaaacgcaacaacctattttgctcgctctctatcaaaaacactggaaaaaggtttatgcggtagatttacgtaaagtcaagctggagcaagtcaaagaatggcttctcagatttaacgtaataagtggctgggggcttaactatgaccttgtcaggcttggtttttcttatgaagagttaaaagaccatgtcgtgttagacttgcttttacttgcacgggaaaagctttacaaaagagacagctttaagttagatgatgtgttaaaagatgtgcttggagtggagtatccatttgacaaaacgaaaatcagaaaaacattcaaaaacacactttactttacacaagaacagctacaatatgttggtcttgatgtttattatttgcctaaactgtttgatgccatttctgatgatagtttaagcatagttcaacagttagaccaagaagctttaaaagtttgtgtggatacaagccaaagaggaatgccgttcttggttgaagaagcgagagcaaagctaattgttctacgacaagaacttgatgttataactaaagagcttggctttaatccacgctccccgcaacaaactaaaaacgctttatcggttcaagatacaagggaagagacgcttcaagacctaataattaacaatggagtaaagaaggaaatagcggagaaggttttacttgcaagaaagatagctaaagaaatttctatgcttgagacgtatatacagcacggagttagagtgaaaggcatcttttggacaacacaggctccaagcggacgtatgagttgcaatgatgaaaaccttcagcaaattccaagaagtttgagagacttatttggctttactgaagataacgataaagtccttataacagctgactttccgcagatagaattgagattggcaggtgctttatggcgtgagccgaaatttgtagaagcgtttagaaaaggtgaagaccttcataaaataacggcttcaattatctatggcgtgcctgtagatgaagtatctaaggagcaaaggcaggtggcaaagtctgccaattttggactaatttacggagcttctccacaaggtttccaaagatattgcataagcaatggaattccgatggatttagagacggcacaactaattcacacgaaattctttgagacttatacaaagatagctaaagaacatgagcttgtcagagattactttagatacaacacagaagctgaaggtgaaacttggcttggtagaaaatacgtagccaaatctccacagcaaatgcttaactatcaaatacaaggctcaggtgcagagttgtttaagaaaacgatagtagagcttaagaagaagtatccaagccttgctattgttaatttggtgcatgacgagatagtgattgaagcagacagacaaacagcagaggatatagctttaattgtaaaagcggagatggaacaagcttgggagtggtgtttagaagaagctaaacaacaaggtaggttgattgaagaatttaaacttgaagttgaaatgcctaacattagcaaaaaatgggaaaaaccataaaggaggtagcaccatgataaaagtctggttggaccattttcttttgtatgttgctgacccagacaaacttccagccattttggacgaagccgatttcagtaaagctttaataccacgaaagatttggaagactgtttatcccattatgcagaaaaagggctttgctatagcaagagtagtgttaatagaggaggagccttacgatgaatggacttgtgagctatgaacgtgtaagatgtaataagcttatcagatgcaaatggttagacgagcaaaaagaactcattattacgctttacgaaatcggctttactatcagagaaatagcgacatatttcaaagttagcgatactccaatcattgatagactgctggagtggggcgtcaagctccgctctggcaacaaactaaaagatattcaagtggatgagtattacggcgaatgctttgataaaatactaagaaagctcatggaaataaggaggaagcgactgctaa

SEQ ID NO: 37 - protein, 840 aa, Unknown
  Clone 1160 from uncultured newly isolated virus
ERIDKRIIEDQARKFANIKLLQDTLIREGYKDQKVKLLSAIDNLLSRILFALRIFTDVLQIPQEEWEELLTQIALYVDTSLSTFYKLFLPREKRLEEELVDFLTQLTDILYKTINDRNRPDLPRTLGGASLDKLIKIAKVQIPSSQVLKYSKDVLLKKYKTARAYLFASSVLVSRIDDAELINNDVERLLENVNNMPDKDRDLCLYAYINTAKRVLSEKVFNSVYTVLKSKGVDVDKYLNMSFVDDDDDTEPPPISPNNNGGADNEPQEDNRFAEIKEVKQLTAPLPDVELITDFKQIKDHIQYDGTIYIDVEADVETQQPILLALYQKHWKKVYAVDLRKVKLEQVKEWLLRFNVISGWGLNYDLVRLGFSYEELKDHVVLDLLLLAREKLYKRDSFKLDDVLKDVLGVEYPFDKTKIRKTFKNTLYFTQEQLQYVGLDVYYLPKLFDAISDDSLSIVQQLDQEALKVCVDTSQRGMPFLVEEARAKLIVLRQELDVITKELGFNPRSPQQTKNALSVQDTREETLQDLIINNGVKKEIAEKVLLARKIAKEISMLETYIQHGVRVKGIFWTTQAPSGRMSCNDENLQQIPRSLRDLFGFTEDNDKVLITADFPQIELRLAGALWREPKFVEAFRKGEDLHKITASIIYGVPVDEVSKEQRQVAKSANFGLIYGASPQGFQRYCISNGIPMDLETAQLIHTKFFETYTKIAKEHELVRDYFRYNTEAEGETWLGRKYVAKSPQQMLNYQIQGSGAELFKKTIVELKKKYPSLAIVNLVHDEIVIEADRQTAEDIALIVKAEMEQAWEWCLEEAKQQGRLIEEFKLEVEMPNISKKWEKP

SEQ ID NO: 38 - DNA, 2906 bp, Unknown
  Clone 1440 from uncultured newly isolated virus
gaggtagagcaaacacagaagagaaaagaaagcaagttgaaaatctcatctatctgatagcaaatcaaggcgtaaaagctcattcttatggtgtatttgaaccaattgatgtgcctgttgttatcgctggagagcctcagaacttacctgttgaggcaatgcttcaggaaaacgaaggtctcttcagaaggtctttagttatatctcttgacgacaggaacttgaaaaagtatgaaaagctaatgagcttttacaacgagctgatagaacaattttacaaccatcatggtttcgcttataagataattgaagacttagaaaggatagacaaacgcattattgaggagcaagcacgtaagttcgcaagtattaagttgcttcaagacacactgataagagaaggttataaagaccaaaaggtaaagcttctttctgctattgacaacttactttcaagaatacttttcgctcttagaattttcactgatgttcttcaaattccgcaagaagagtgggaagagcttttgacacaaatagctttatacgtggatacaagcttatcaacattctataaactattcctgccacgtgagaaaaggcttgaagaagagcttgtagattttctgacacaattgactgatattctttacaagactatcaacgatagaaacagaccagacttgcctcggactttgggaggagcttctttggataagcttatcaaaatagcaaaggtgcaaataccaagctcgcaagtcttgaagtattctaaagatgttcttctcaagaaatacaaaacagcaagagcttatttattcgcaagctctgtgcttgttagcagaattgatgatgcagaattgataaacaacgacgtggagagattgttagaaaatgtaaataacatgcctgacaaagatagagacctttgcctttatgcttatatcaatactgcaaaacgtgttttgagtgagaaagtgtttaactctgtttataccgtattgaaaagcaaaggtgtagatgtggataagtatttgaatatgagctttgtagatgacgacgatgacacagaaccaccaccaattggcggtaataataccgaaccaccgaataacaatggcggtgctgataacgaaccgcaagaagataatcgttttgttgaaattaaagaagtcaaacaactaaccgcacctctgcctgatgtagagcttataacagactttaagcaaattaaagaccacatacaatacgatggaactatatacatagacgtagaagcagatgttgaaacgcaacaacctattttgctcgctctctatcaaaaacactggaaaaaggtttatgcggtagatttacgtaaagtcaagctggagcaagtcaaagaatggcttctcagatttaacgtaataagtggctgggggcttaactatgaccttgtcaggcttggtttctcttatgaagagttaaaagaccatgtcgtgttagacttgcttttacttgcacgggaaaagctttacacaagagacagctttaagttagatgatgtgttaaaagatgtgcttggagtggagtatccatttgacaaaacgaaaatcagaaaaacattcaaaaacacactttactttacacaagaacagctacaatatgcaggtttggatgtttattatttacctaaactgtttgatgccatttctgatgacagtttaagcatagttcaacagttagaccaagaagctttaaaggtttgtgtggatacaagccaaagaggaatgccgttcttggttgaagaagcgagagcaaagctaattgttctacgacaagaacttgatgttataaataaagaacttggctttaatccacgctccccgcaacaaactaaaaacgctttatcggttcaagatacgagggaagagacgcttcaagacctaataatcaacaatggagtaaagaaggagatagcggagaaggttttacttgcaagaaagatagctaaagaaatttctatgcttgagacgtatatacagcacggagttagagtgaaaggcatcttttggacaacacaggctccaagcggacgtatgagttgcaatgatgaaaatcttcagcaggttccaagaagtttgagagacttgtttggctttactgaagataacgataaagtccttataacagctgactttccgcagatagaattgagactggcaggtgctttatggcgtgagccgaaatttgtagaagcgtttagaaaaggtgaagaccttcataaaataacggcttcaattatctatggcgtgcctgtagatgaagtatctaaggagcaaaggcaggtggcaaagtctgctaattttggactaatttacggagcttctccacaaggtttccaaagatattgcataagcaatggaattccgatggatttagagacggcacaactaattcacacgaaattctttgagacttatacaaagatagctaaagaacatgagcttgtcaaagattactttagatacaacacagaagctgaaggtgaaacttggcttggtagaaaatacgtagctaaatctccacagcaaatgcttaactatcaaatacaaggttcaggtgcagagttgtttaagaagacgatagtagagcttaagaagaagtatccaagccttgctattgttaatttggtgcatgacgagatagtgattgaagcagacagacaaacagcagaggatatagctttaattgtaaaagcggagatggaacaagcttgggagtggtgtttagaagaagctaaacagcaaggtaggttgattgaagaatttaaacttgaagttgaaatgcctaacattagcaaaaaatgggaaaaaccataaaggaggtcatatgtatatctccttcttatagttaaacaaaattatttc

SEQ ID NO: 39 - protein, 951 aa, Unknown
  Clone 1440 from uncultured newly isolated virus
GRANTEEKRKQVENLIYLIANQGVKAHSYGVFEPIDVPVVIAGEPQNLPVEAMLQENEGLFRRSLVISLDDRNLKKYEKLMSFYNELIEQFYNHHGFAYKIIEDLERIDKRIIEEQARKFASIKLLQDTLIREGYKDQKVKLLSAIDNLLSRILFALRIFTDVLQIPQEEWEELLTQIALYVDTSLSTFYKLFLPREKRLEEELVDFLTQLTDILYKTINDRNRPDLPRTLGGASLDKLIKIAKVQIPSSQVLKYSKDVLLKKYKTARAYLFASSVLVSRIDDAELINNDVERLLENVNNMPDKDRDLCLYAYINTAKRVLSEKVFNSVYTVLKSKGVDVDKYLNMSFVDDDDDTEPPPIGGNNTEPPNNNGGADNEPQEDNRFVEIKEVKQLTAPLPDVELITDFKQIKDHIQYDGTIYIDVEADVETQQPILLALYQKHWKKVYAVDLRKVKLEQVKEWLLRFNVISGWGLNYDLVRLGFSYEELKDHVVLDLLLLAREKLYTRDSFKLDDVLKDVLGVEYPFDKTKIRKTFKNTLYFTQEQLQYAGLDVYYLPKLFDAISDDSLSIVQQLDQEALKVCVDTSQRGMPFLVEEARAKLIVLRQELDVINKELGFNPRSPQQTKNALSVQDTREETLQDLIINNGVKKEIAEKVLLARKIAKEISMLETYIQHGVRVKGIFWTTQAPSGRMSCNDENLQQVPRSLRDLFGFTEDNDKVLITADFPQIELRLAGALWREPKFVEAFRKGEDLHKITASIIYGVPVDEVSKEQRQVAKSANFGLIYGASPQGFQRYCISNGIPMDLETAQLIHTKFFETYTKIAKEHELVKDYFRYNTEAEGETWLGRKYVAKSPQQMLNYQIQGSGAELFKKTIVELKKKYPSLAIVNLVHDEIVIEADRQTAEDIALIVKAEMEQAWEWCLEEAKQQGRLIEEFKLEVEMPNISKKWEKP

SEQ ID NO: 40 - DNA, 2876 bp, Unknown
  Clone 1128 from uncultured newly isolated virus
gctgcgagcgtcttgagctgatagaacaattttacaaccatcatggtttcgcttataagataattgaagacttagaaaggatagacaaacgcatcattgaggagcaagcacgtaagttcgcaagtattaagttgcttcaggacacactgataagagaaggctataaagaccaaaaggtaaagcttctttctgctattgacaacttactttcaagaatacttttcgctcttagaattttcactgatgttcttcaaattccgcaagaagagtgggaagagcttttcacacaaatagctttatacgtggatacaagcttatcaacattctacaaactatttttaccacgtgagaaaaggcttgaagaagagcttgtagattttctgacacaattgactgatattctttacaagactatcaacgatagaaacagaccagacttgcctcggactttgggaggagcttctttggataagcttatcaaaatagcaaaggtgcaaataccaagctcgcaagtcttgaagtattctaaagacgttcttctcaagaaatacaaaacagcaagagcttatttattcgcaagctctgtgcttgttagcagaattgatgatgcagaattgataaacaacgacgtggagagattgttagaaaatgtaaataacatgcctgacaaagatagagacctttgcctttatgcttatatcaatactgcaaaacgtgttttgagtgagaaagtgtttaactctgtttataccgtattgaaaagcaaaggtgtagatgtggataagtatttgaatatgagctttgtagatgacgacgatgacacagaaccaccaccaattggcggtaataataccgaaccaccgaataacaatggcggtgctgataacgaaccgcaagaagataatcgttttgttgaaattaaagaagtcaaacaactaaccgcacctctgcctgatgtagagcttataacagactttaagcaaattaaagaccacatacaatacgatggaactatatacatagacgtagaagcagatgttgaaacgcaacaacctattttgctcgctctctatcaaaaacactggaaaaaggtttatgcggtagatttacgtaaagtcaagctggagcaagtcaaagaatggcttctcagatttaacgtaataagtggctgggggcttaactatgaccttgtcaggcttggtttctcttatgaagagttaaaagaccatgtcgtgttagacttgcttttacttgcacgggaaaagctttacaaaagagacagctttaagttagatgatgtgttaaaagatgtgcttggagtggagtatccatttgacaaaacgaaaatcagaaaaacattcaaaaacacactttactttacacaagaacagctacaatatgttggtcttgatgtttattatttgcctaaactgtttgatgccatttctgatgacagtttaagcatagttcaacagttagaccaagaagctttaaaggtttgtgtggatacaagccaaagaggaatgccgttcttggttgaagaagcgagagcaaagctaattgttctacgacaagaacttgatgttataaccaaagagcttggctttaatccacgctccccgcaacaaactaaaaacgctttatcggttcaagatacgagggaagagacgcttcaagacctaataatcaacaatggagtaagaaaggaaatagcggagaaggttttacttgcaagaaagatagctaaagaaatttctatgcttgagacgtatatacagcacggagttagagtgaaaggcatcttttggacaacacaggctccaagcggacgtatgagttgcaatgatgaaaatcttcagcaggttccaagaagtttgagagacttgtttggctttactgaagataacgataaagtccttataacagctgactttccgcagatagaattgagactggcaggtgctttatggcgtgagccgaaatttgtagaagcgtttagaaaaggtgaagaccttcataaaataacggcttcaattatctatggcgtgcctgtagatgaagtatctaaggagcaaaggcaggtggcaaagtctgccaattttggactaatttacggagcttctccacaaggtttccaaagatattgcataagcaatggaattccgatggatttagagacggcacaactaattcacacgaaattctttgagacttatacaaagatagctaaagaacatgagcttgtcaaagattactttagatacaacacagaagctgaaggtgaaacttggcttggtagaaaatacgtagctaaatctccacagcaaatgcttaactatcaaatacaaggttcaggtgcagagttgtttaagaagacgatagtagagcttaagaagaagtatccaagccttgctattgttaatttggtgcatgacgagatagtgattgaagcagacagacaaacagcagaggatatagctttaattgtaaaagcggagatggaacaagcttgggagtggtgtttagaagaagctaaacagcaaggtaggttgattgaagaatttaaacttgaagttgaaatgcctaacattagcaaaaaatgggaaaaaccataaaggaggtggcaccatgataaaagtctggttggaccattttcttttgtatgttgctgacccagacaaacttccagccattttggacgaagccgatttcagtaaagctttaataccacgaaagatttggaagactgtttatcccattatgcagaaaaagggctttgctatagcaagagtagtgttaatagaggaggagccttacgatgaatggacttgtgagacatatgtatattccttcttatagttaaacaaaattatttc

SEQ ID NO: 41 - protein, 870 aa, Unknown
  Clone 1128 from uncultured newly isolated virus
CERLELIEQFYNHHGFAYKIIEDLERIDKRIIEEQARKFASIKLLQDTLIREGYKDQKVKLLSAIDNLLSRILFALRIFTDVLQIPQEEWEELFTQIALYVDTSLSTFYKLFLPREKRLEEELVDFLTQLTDILYKTINDRNRPDLPRTLGGASLDKLIKIAKVQIPSSQVLKYSKDVLLKKYKTARAYLFASSVLVSRIDDAELINNDVERLLENVNNMPDKDRDLCLYAYINTAKRVLSEKVFNSVYTVLKSKGVDVDKYLNMSFVDDDDDTEPPPIGGNNTEPPNNNGGADNEPQEDNRFVEIKEVKQLTAPLPDVELITDFKQIKDHIQYDGTIYIDVEADVETQQPILLALYQKHWKKVYAVDLRKVKLEQVKEWLLRFNVISGWGLNYDLVRLGFSYEELKDHVVLDLLLLAREKLYKRDSFKLDDVLKDVLGVEYPFDKTKIRKTFKNTLYFTQEQLQYVGLDVYYLPKLFDAISDDSLSIVQQLDQEALKVCVDTSQRGMPFLVEEARAKLIVLRQELDVITKELGFNPRSPQQTKNALSVQDTREETLQDLIINNGVRKEIAEKVLLARKIAKEISMLETYIQHGVRVKGIFWTTQAPSGRMSCNDENLQQVPRSLRDLFGFTEDNDKVLITADFPQIELRLAGALWREPKFVEAFRKGEDLHKITASIIYGVPVDEVSKEQRQVAKSANFGLIYGASPQGFQRYCISNGIPMDLETAQLIHTKFFETYTKIAKEHELVKDYFRYNTEAEGETWLGRKYVAKSPQQMLNYQIQGSGAELFKKTIVELKKKYPSLAIVNLVHDEIVIEADRQTAEDIALIVKAEMEQAWEWCLEEAKQQGRLIEEFKLEVEMPNISKKWEKP

SEQ ID NO: 42 - DNA, 2982 bp, Unknown
  Clone 1753 from uncultured newly isolated virus
actataagaaggagatatacatatggtgcctgttgttatcgctggagagcctcagaacttacctgttgaggcaatgcttcaggaaaacgaaggtctcttcagaaggtctttagttatatctcttgacgacaggaatttgaaaaagtatgaaaagctaatgagcttttacaacgagctgatagaacaattttacaaccatcatggtttcgcttataagataattgaagacttagaaaggatagacaaacgcatcattgaggaccaagcacgtaagtttgcaaatattaagttgcttcaggacacactgataagagaaggttataaagaccaaaaggtaaagcttctttctgctattgacaacttactttcaagaatacttttcgctcttagaattttcactgatgttcttcaaattccgcaagaagagtgggaagagcttttgacacaaatagctttatacgtggatacaagcttatcaacattctataaactattcctgccacgtgagaaaaggcttgaagaagagcttgtagattttctgacacaattgactgatattctttacaagactatcaacgatagaaacagaccagacttgcctcggactttgggaggagcttctttggataagcttatcaaaatagcaaaggtgcaaataccaagctcgcaagtcttgaagtattctaaagatgttcttctcaagaaatacaaaacagcaagagcttatttattcgcaagctctgtgcttgttagcagaattgatgatgcagaattgataaacaacgacgtggagagattgttagaaaatgtaaataacatgcctgacaaagatagagacctttgcctttatgcttatatcaatactgcaaaacgtgttttgagtgagaaagtgtttaactctgtttataccgtattgaaaagcaaaggtgtagatgtggataagtatttgaatatgagctttgtagatgacgacgatgacacagaaccaccaccaatatcaccgaataacaatggtggtgctgataacgaaccgcaagaagataatcgttttgctgaaattaaagaagtcaagcaactaaccgcacctctgcctgatgtagagcttataacagactttaagcaaattaaagaccacatacaatacgatggaactatatacatagacgtagaagcagatgttgaaacgcaacaacctattttgctcgctctctatcaaaaacactggaaaaaggtttatgcggtagatttacgtaaagtcaagctggagcaagtcaaagaatggcttctcagatttaacgtaataagtggctgggggcttaactatgaccttgtcaggcttggtttttcttatgaagagttaaaagaccatgtcgtgttagacttgcttttacttgcacgggaaaagctttacaaaagagacagctttaagttagatgatgtgttaaaagatgtgcttggagtggagtatccatttgacaaaacgaaaatcagaaaaacattcaaaaacacactttactttacacaagaacagctacaatatgttggtcttgatgtttattatttgcctaaactgtttgatgccatttctgatgatagtttaagcatagttcaacagttagaccaagaagctttaaaagtttgtgtggatacaagccaaagaggaatgccgttcttggttgaagaagcgagagcaaagctaattgttctacgacaagaacttgatgttataactaaagagcttggctttaatccacgctccccgcaacaaactaaaaacgctttatcggttcaagatacaagggaagagacgcttcaagacctaataattaacaatggagtaaagaaggaaatagcggagaaggttttacttgcaagaaagatagctaaagaaatttctatgcttgagacgtatatacagcacggagttagagtgaaaggcatcttttggacaacacaggctccaagcggacgtatgagttgcaatgatgaaaaccttcagcaaattccaagaagtttgagagacttatttggctttactgaagataacgataaagtccttataacagctgactttccgcagatagaattgagattggcaggtgctttatggcgtgagccgaaatttgtagaagcgtttagaaaaggtgaagaccttcataaaataacggcttcaattatctatggcgtgcctgtagatgaagtatctaaggagcaaaggcaggtggcaaagtctgccaattttggactaatttacggagcttctccacaaggtttccaaagatattgcataagcaatggaattccgatggatttagagacggcacaactaattcacacgaaattctttgagacttatacaaagatagctaaagaacatgagcttgtcagagattactttagatacaacacagaagctgaaggtgaaacttggcttggtagaaaatacgtagccaaatctccacagcaaatgcttaactatcaaatacaaggctcaggtgcagagttgtttaagaaaacgatagtagagcttaagaagaagtatccaagccttgctattgttaatttggtgcatgacgagatagtgattgaagcagacagacaaacagcagaggatatagctttaattgtaaaagcggagatggaacaagcttgggagtggtgtttagaagaagctaaacaacaaggtaggttgattgaagaatttaaacttgaagttgaaatgcctaacattagcaaaaaatgggaaaaaccataaaggaggtagcaccatgataaaagtctggttggaccattttcttttgtatgttgctgacccagacaaacttccagccattttggacgaagccgatttcagtaaagctttaataccacgaaagatttggaagactgtttatcccattatgcagaaaaagggctttgctatagcaagagtagtgttaatagaggaggagccttacgatgaatggacttgtgagctatgaa

SEQ ID NO: 43 - protein, 910 aa, Unknown
  Clone 1753 from uncultured newly isolated virus
MVPVVIAGEPQNLPVEAMLQENEGLFRRSLVISLDDRNLKKYEKLMSFYNELIEQFYNHHGFAYKIIEDLERIDKRIIEDQARKFANIKLLQDTLIREGYKDQKVKLLSAIDNLLSRILFALRIFTDVLQIPQEEWEELLTQIALYVDTSLSTFYKLFLPREKRLEEELVDFLTQLTDILYKTINDRNRPDLPRTLGGASLDKLIKIAKVQIPSSQVLKYSKDVLLKKYKTARAYLFASSVLVSRIDDAELINNDVERLLENVNNMPDKDRDLCLYAYINTAKRVLSEKVFNSVYTVLKSKGVDVDKYLNMSFVDDDDDTEPPPISPNNNGGADNEPQEDNRFAEIKEVKQLTAPLPDVELITDFKQIKDHIQYDGTIYIDVEADVETQQPILLALYQKHWKKVYAVDLRKVKLEQVKEWLLRFNVISGWGLNYDLVRLGFSYEELKDHVVLDLLLLAREKLYKRDSFKLDDVLKDVLGVEYPFDKTKIRKTFKNTLYFTQEQLQYVGLDVYYLPKLFDAISDDSLSIVQQLDQEALKVCVDTSQRGMPFLVEEARAKLIVLRQELDVITKELGFNPRSPQQTKNALSVQDTREETLQDLIINNGVKKEIAEKVLLARKIAKEISMLETYIQHGVRVKGIFWTTQAPSGRMSCNDENLQQIPRSLRDLFGFTEDNDKVLITADFPQIELRLAGALWREPKFVEAFRKGEDLHKITASIIYGVPVDEVSKEQRQVAKSANFGLIYGASPQGFQRYCISNGIPMDLETAQLIHTKFFETYTKIAKEHELVRDYFRYNTEAEGETWLGRKYVAKSPQQMLNYQIQGSGAELFKKTIVELKKKYPSLAIVNLVHDEIVIEADRQTAEDIALIVKAEMEQAWEWCLEEAKQQGRLIEEFKLEVEMPNISKKWEKP

SEQ ID NO: 44 - DNA, 3743 bp, Unknown
  Clone 1773 from uncultured newly isolated virus
actataagaaggagatatacatatgggaacaggtaagactacaagactaaaggtagcttcggctttgtatggcttaccgcttactatagatatctcggaaacgacgataacaagaatagagcgtgagtttggaaactacaaagttccgcttccgcttgacgaagtaagagcaaacacagaagagaaaagaaagcaagttgaaaatctcatttatctgatagcaaatcaaggcgtaaaagctcattctcatggtgtgtttgaaccaatagatgtgcctgttgttatcgctggagagcctcagaacttacctgttgaggcaatgcttcaggaaaacgaaggtctcttcagaaggtctttagttatatctcttgacgacaggaatttgaaaaagtatgaaaagctaatgagcttttacaacgagctgatagaacaattttacaaccatcacggcttcgcttataagataattgaagacttagaaaggatagacaaacgcatcattgaggagcaagcacgtaagttcgcaagtattaagttgcttcaggacacactgataagagaaggctataaagaccaaaaggtaaagcttctttctgctattgacaacttactttcaagaatacttttcgctcttagaattttcactgatgttcttcaaattccgcaagaagagtgggaagagcttttgacacaaatagctttatacgtggatacaagcttatcaacattctacaaactatttttaccacgtgagaaaaggcttgaagaagagcttgtagattttctgacacaattgactgatattctttacaagactatcaacgatagaaacagaccagacttgcctcggactttgggaggagcttctttggataagcttatcaaaatagcaaaggtgcaaataccaagctcgcaagtcttgaagtattctaaagacgttcttctcaagaaatacaaaacaacaagagcttatttattcgcaagctctgtgcttgttagcagaattgatgatgcagaattgataaacaacgacgtggagagattgttagaaaatgtaaataacatgcctgacaaagatagagacctttgtctctatgcttatatcaataccgcaaaacgtgttttgagtgagaaagtgtttaactctgtttataccgtattaaaaggcaaaggtgtagatgtagaaaagtatttgaatatgagctttgcagatgatgacgatgacgcagaaccgccaccaattggcggtaataataccgaaccaccgaataacaatggtggtgctgataacgaaccgcaagaagataatcgttttgctgaaattaaagaagtcaaacaactaaccgcacctctacctgatgtagagcttataacagactttaagcaaattaaagaccacatacaatacgatggaactatatacatagacgtagaagcagatgttgaaacgcaacaacctattttgctcgctctctatcaaaaacactggaaaaaggtttatgcggtagatttacgtaaagtcaagctggagcaagtcaaagaatggcttctcagatttaacgtaataagtggctgggggcttaactatgaccttgtcaggcttggtttctcttatgaagagttaaaagaccatgtcgtgttagacttgcttttacttgcacgggaaaagctttacaaaagagacagctttaagttagatgatgtgttaaaagatgtgcttggagtggagtatccatttgacaaaacgaaaatcagaaaaacattcaaaaacacactttactttacacaagaacagctacaatatgttggtcttgatgtttattatttgcctaaactgtttgatgccatttctgatgacagtttaagcatagttcaacagttagaccaagaagctttaaaggtttgtgtggatacaagccaaagaggaatgccgttcttggttgaagaagcgagagcaaagctaattgttctacgacaagaacttgatgttataaccaaagagcttggctttaatccacgctccccgcaacaaactaaaaacgctttatcggttcaagatacgagggaagagacgcttcaagacctaataatcaacaatggagtaagaaaggaaatagcggagaaggttttacttgcaagaaagatagctaaagaaatttctatgcttgagacgtatatacagcacggagttagagtgaaaggcatcttttggacaacacaggctccaagcggacgtatgagttgcaatgatgaaaatcttcagcaggttccaagaagtttgagagacttgtttggctttactgaagataacgataaagtccttataacagctgactttccgcagatagaattgagactggcaggtgctttatggcgtgagccgaaatttgtagaagcgtttagaaaaggtgaagaccttcataaaataacggcttcaattatmtatggcgtgcctgtagatgaagtatctaaggagcaaaggcaggtggcaaagtctgccaattttggactaatttacggagcttctccacaaggtttccaaagatattgcataagcaatggaattccgatggatttagagacggcacaactaattcacacgaaattctttgagacttatacaaagatagctaaagaacatgagcttgtcaaagattactttagatacaacacagaagctgaaggtgaaacttggcttggtagaaaatacgtagctaaatctccacagcaaatgcttaactatcaaatacaaggttcaggtgcagagttgtttaagaagacgatagtagagcttaagaagaagtatccaagccttgctattgttaatttggtgcatgacgagatagtgattgaagcagacagacaaacagcagaggatatagctttaattgtaaaagcggagatggaacaagcttgggagtggtgtttagaagaarctaaacagcaaggtaggttgattgaagaatttaaacttgaagttgaaatgcctaacattagcaaaaaatgggaaaaaccataaaggaggtggcaccatgataaaagtctggttggaccattttcttttgtatgttgctgacccagacaaacttccagccattttggacgaagccgatttcagtaaagctttaataccacgaaagatttggaagactgtttatcccattatgcagaaaaagggctttgctatagcaagagtagtgttaatagaggaggagccttacgatgaatggacttgtgagatatgaacgtgtaagatgtaataagcttatcagatgcaaatggttagacgagcaaaaagaactcattattacgctttacgaaattggctttactatcagagaaatagcaacatatttcaaagttagcgatactccaatcattgatagactgctggagtggggcgtcaagctccgctctggcaacaaactaaaagatattcaagtggatgagtattacggcgaatgctttgataaaatactaagaaagctcatggaaataaggaggaagcgacttgctaaagctcgttctaatcaaacaaaacagcgatataaaaaaggaggagatagatatgagtattgatgattttatcaaaaaacacaacttagaacatgttatagagaaagcggtagagatagttaagaactactttccagatgccgaaatacaattttatctttaccaagaccacgagatagaagatttacaaacacttattattggtataaatatgactaatacagcga

SEQ ID NO: 45 - protein, 998 aa, Unknown
  Clone 1773 from uncultured newly isolated virus
MGTGKTTRLKVASALYGLPLTIDISETTITRIEREFGNYKVPLPLDEVRANTEEKRKQVENLIYLIANQGVKAHSHGVFEPIDVPVVIAGEPQNLPVEAMLQENEGLFRRSLVISLDDRNLKKYEKLMSFYNELIEQFYNHHGFAYKIIEDLERIDKRIIEEQARKFASIKLLQDTLIREGYKDQKVKLLSAIDNLLSRILFALRIFTDVLQIPQEEWEELLTQIALYVDTSLSTFYKLFLPREKRLEEELVDFLTQLTDILYKTINDRNRPDLPRTLGGASLDKLIKIAKVQIPSSQVLKYSKDVLLKKYKTTRAYLFASSVLVSRIDDAELINNDVERLLENVNNMPDKDRDLCLYAYINTAKRVLSEKVFNSVYTVLKGKGVDVEKYLNMSFADDDDDAEPPPIGGNNTEPPNNNGGADNEPQEDNRFAEIKEVKQLTAPLPDVELITDFKQIKDHIQYDGTIYIDVEADVETQQPILLALYQKHWKKVYAVDLRKVKLEQVKEWLLRFNVISGWGLNYDLVRLGFSYEELKDHVVLDLLLLAREKLYKRDSFKLDDVLKDVLGVEYPFDKTKIRKTFKNTLYFTQEQLQYVGLDVYYLPKLFDAISDDSLSIVQQLDQEALKVCVDTSQRGMPFLVEEARAKLIVLRQELDVITKELGFNPRSPQQTKNALSVQDTREETLQDLIINNGVRKEIAEKVLLARKIAKEISMLETYIQHGVRVKGIFWTTQAPSGRMSCNDENLQQVPRSLRDLFGFTEDNDKVLITADFPQIELRLAGALWREPKFVEAFRKGEDLHKITASIXYGVPVDEVSKEQRQVAKSANFGLIYGASPQGFQRYCISNGIPMDLETAQLIHTKFFETYTKIAKEHELVKDYFRYNTEAEGETWLGRKYVAKSPQQMLNYQIQGSGAELFKKTIVELKKKYPSLAIVNLVHDEIVIEADRQTAEDIALIVKAEMEQAWEWCLEEXKQQGRLIEEFKLEVEMPNISKKWEKP

SEQ ID NO: 46 - DNA, 1930 bp, Unknown
  Clone 1937 from uncultured newly isolated virus
gtgagaaagtgtttaactctgtttataccgtattgaaaagcaaaggtgtagatgtggataagtatttgaatatgagctttgtagatgacgacgatgacacagaaccaccaccaattggcggtaataataccgaaccaccgaataacaatggcggtgctgataacgaaccgcaagaagataatcgttttgttgaaattaaagaagtcaaacaactaaccgcacctctgcctgatgtagagcttataacagactttaagcaaattaaagaccacatacaatacgatggaactatatacatagacgtagaagcagatgttgaaacgcaacaacctattttgctcgctctctatcaaaaacactggaaaaaggtttatgcggtagatttacgtaaagtcaagctggagcaagtcaaagaatggcttctcagatttaacgtaataagtggctgggggcttaactatgaccttgtcaggcttggtttctcttatgaagagttaaaagaccatgtcgtgttagacttgcttttacttgcacgggaaaagctttacaaaagagacagctttaagttagatgatgtgttaaaagatgtgcttggagtggagtatccatttgacaaaacgaaaatcagaaaaacattcaaaaacacactttactttacacaagaacagctacaatatgttggtcttgatgtttattatttgcctaaactgtttgatgccatttctgatgacagtttaagcatagttcaacagttagaccaagaagctttaaaggtttgtgtggatacaagccaaagaggaatgccgttcttggttgaagaagcgagagcaaagctaattgttytacgacaagaacttgatgttataaccaaagagcttggctttaatccacgctccccgcaacaaactaaaaacgctttatcggttcaagatacgagggaagagacgcttcaagacctaataatcaacaatggagtaagaaaggaaatagcggagaaggttttacttgcaagaaagatagctaaagraatttctatgcttgagacgtatatacagcacggagttagagtgaaaggcatcttttggacaacacaggctccaagcggacgtatgagttgcaatgatgaaaatcttcagcaggttccaagaagtttgagagacttgtttggctttactgaagataacgataaagtccttataacagctgactttccgcagatagaattgagactggcaggtgctttatggcgtgagccgaaatttgtagaagcgtttagaaaaggtgaagaccttcataaaataacggcttcaattatctatggcgtgcctgtagatgaagtatctaaggagcaaaggcaggtggcaaagtctgccaattttggactaatttacggagcttctccacaaggtttccaaagatattgcataagcaatggaattccgatggatttagagacggcacaactaattcacacgaaattctttgagacttatacaaagatagctaaagaacatgagcttgtcaaagattactttagatacaacacagaagctgaaggtgaaacttggcttggtagaaaatacgtagctaaatctccacagcaaatgcttaactatcaaatacaaggttcaggtgcagagttgtttaagaagacgatagtagagcttaagaagaagtatccaagccttgctattgttaatttggtgcatgacgagatagtgattgaagcagacagacaaacagcagaggatatagctttaattgtaaaagcggagatggaacaagcttgggagtggtgtttagaagaagctaaacagcaaggtaggttgattgaagaatttaaacttgaagttgaaatgcctaacattagcaaaaaatgggaaaaagtatgaaaagctgatgagcttttacaacgagcgagacgctcgcg

SEQ ID NO: 47 - protein, 629 aa, Unknown
  Clone 1937 from uncultured newly isolated virus
EKVFNSVYTVLKSKGVDVDKYLNMSFVDDDDDTEPPPIGGNNTEPPNNNGGADNEPQEDNRFVEIKEVKQLTAPLPDVELITDFKQIKDHIQYDGTIYIDVEADVETQQPILLALYQKHWKKVYAVDLRKVKLEQVKEWLLRFNVISGWGLNYDLVRLGFSYEELKDHVVLDLLLLAREKLYKRDSFKLDDVLKDVLGVEYPFDKTKIRKTFKNTLYFTQEQLQYVGLDVYYLPKLFDAISDDSLSIVQQLDQEALKVCVDTSQRGMPFLVEEARAKLIVXRQELDVITKELGFNPRSPQQTKNALSVQDTREETLQDLIINNGVRKEIAEKVLLARKIAKXISMLETYIQHGVRVKGIFWTTQAPSGRMSCNDENLQQVPRSLRDLFGFTEDNDKVLITADFPQIELRLAGALWREPKFVEAFRKGEDLHKITASIIYGVPVDEVSKEQRQVAKSANFGLIYGASPQGFQRYCISNGIPMDLETAQLIHTKFFETYTKIAKEHELVKDYFRYNTEAEGETWLGRKYVAKSPQQMLNYQIQGSGAELFKKTIVELKKKYPSLAIVNLVHDEIVIEADRQTAEDIALIVKAEMEQAWEWCLEEAKQQGRLIEEFKLEVEMPNISKKWEKV

SEQ ID NO: 48 - DNA, 1758 bp, Hydrogenobaculum sp.
atgaattttgtgtatgtagataaagagcctgttttaataaaagctttagactatctttcctctggtgatatttggtttatagacacagaaaccacgccaaaagatataagactttttcaagtaggattagagagcggtcctatttatgtgatagactttttgtttgtaaaaagagctccagaacttataaaagatataatagctaaaaagggtgtagcaggacacaacttaaaatatgatttaaagtatcttatgaaatacgatatacatccttatactacgtttgatactatggtaggagcacagttgataggtttaaatagagtttctttggcgagtgtttacaatcattttacaggtgaaagtatcgacaaaaaagagcagttttcaaattggtcttcaaaagagcttacagaaagtcaaattttttatgctgcaaaggatgtagaggttttgaggcttttatacgaaaagctaaaaaatgaattaaacaaagaacccaccatcattgagatattacaaaaatcaagggtggcgaaggtttttggattggaaagcacatacgctataatagaaatggggtttgtacaggagcttgctaaaattgaacacaccggaataggaatagatacaaaagaaatagagactatgaaaaaacaattacaaaagaaaacccaagagcttgctatgaacttttatataaagtatcgtatagatataagtagtcctaaaaaagtaggtgagtttttagaaaatcatttaaatatttcacttcctcgtaccgataaagacaatataataacagatgatagtgtgttgatagagcatcttgactatgaaaacgagaaagcaaaagatgtgataagtagcgtattggagtttagaaagcttcataagttacaagaaaagctatcagagattttagagtacaacgaaaacaaccgtatacatccagagttttggcaaataggagccgttactggaaggatgtcctcttcaagacccaatgttcaaaacataccaagagaattaagaagtattctaaaagctaaagacggatacgtgtttgtaatagctgatttctcacaaatagaactaagaatagcagcagagtatgttaaagatgaggtaatgatagatataataaataaaggagaagaccttcacaagtttacagcctcattaattacaggtaaatcgttggaagatattacaaaagaagaaagacaaagggcaaaagctgccaatttcggtcttatatacggtatatcagaaaaatctctttctttgtatgcaagaaactcttatgggattgatatgtctatagaagaagccaaaagatttagagaggtgtttttttctacattccaagggataaaagcttggcacgaaaggataaaaaaagagctaaaggcaaaaggtgaaataaggttaaaaactatcggcggaaaacctatgatagcctacacttttaccgatgctgccaattatccaatacaaggtactggagcagaattgttgaagctttcagttttaattttttctcaagagcttaaaagagcttttccaagcatatttcacgaagtagcaaacgttgtaaacttggtacacgatgagatagtggtggaagcaaaagaagattataaagaagaagtatctaagcttttagaaaaatctatgaaaaaagctggctctatacttcttaacaatgtaaaaatagaaacagaaatagttatcaatcaccgctggacaaagtaa

SEQ ID NO: 49 - protein, 585 aa, Hydrogenobaculum sp.
MNFVYVDKEPVLIKALDYLSSGDIWFIDTETTPKDIRLFQVGLESGPIYVIDFLFVKRAPELIKDIIAKKGVAGHNLKYDLKYLMKYDIHPYTTFDTMVGAQLIGLNRVSLASVYNHFTGESIDKKEQFSNWSSKELTESQIFYAAKDVEVLRLLYEKLKNELNKEPTIIEILQKSRVAKVFGLESTYAIIEMGFVQELAKIEHTGIGIDTKEIETMKKQLQKKTQELAMNFYIKYRIDISSPKKVGEFLENHLNISLPRTDKDNIITDDSVLIEHLDYENEKAKDVISSVLEFRKLHKLQEKLSEILEYNENNRIHPEFWQIGAVTGRMSSSRPNVQNIPRELRSILKAKDGYVFVIADFSQIELRIAAEYVKDEVMIDIINKGEDLHKFTASLITGKSLEDITKEERQRAKAANFGLIYGISEKSLSLYARNSYGIDMSIEEAKRFREVFFSTFQGIKAWHERIKKELKAKGEIRLKTIGGKPMIAYTFTDAANYPIQGTGAELLKLSVLIFSQELKRAFPSIFHEVANVVNLVHDEIVVEAKEDYKEEVSKLLEKSMKKAGSILLNNVKIETEIVINHRWTK

SEQ ID NO: 50 - DNA, 1764 bp, Sulfurihydrogenibium sp.
atgatggatataaactacatcacacaagaaaatcagcttgaaagtctaaaagtattacaagatacgccttatctgtatttagatacagaagtaatgataaaagattttgaaaacatagattttttcaatgataaaatcaggcttattcaaattggtgatgaagagaatacttttgtcattgacttactaaagataaatcctgaagtggttaaaaatcatattcaaaacctgatagaaaataaaggaattatcggacataacttaaagtttgacttaaaatttttaaaaacaaatttgaacatactcccaaaaatcgtttttgatactatgatagcatctcagatattggcaaaaggagacagcaatcaaaggcattctctatcagcatctgcaaaaagatttgtcagcttagatgtagataaaacatatcaaaaatcaccttggtgggcaaaagacctttcatctgaacagatagaatacgcagcaaaagatatagatacattaagacatctttttaaagaagagaaaaatcaattaaatcaagataatttgcataaaaaggcatcaggagaaacttttaaagtttttggtgtgattaatccggtagcagcacttgaaatggcttttcttccggcactcgtagagattgaactctcaggcattccaatagatgaagaagaggcaaaaaaacttctaaaacaaaaagaatctgaatttcaatctgactatatgaaatttaaaataaaaacaggagcagacccattttctccacagcaggtggtaaactatctaacaaacaaacttaaaatcaaattaccaaaaacagaaaaaggttcgttttcatctcaagatgtattcttaaaagactatgaagatatagaagaagtcagactgcttttaaaactaagagcagacaaaaaaataatcgataaaatcaaagagatattacaatttacaagaaacgaaagggtttacggagagtttaagcagattggagcagcaacaggaagaatgtcatcacttcgtccaaaccttcaaaacataccaaaaaatcttaaatatctgtttaaacctaaggaaggatataagtttatagtggctgactactctcagattgagctaagaattgctgcccagtatacaaaagatgaaaacatgattaccgcctttaatgaaggtaaagacctacacaagcttacagcttcaatcattacaggaaaaagttatgatgaaatcacaaaggaagaaagacaacttgcaaaggctataaactttggtttaatctatggaatgtctccaaagtcgctagtagagtatgcaaaggctaattatggcgtcagtatatctcttcaggaagctaaaaaatttcatgaaaattattttaaattttacaaatcttttaaagattggcatgacaaagttaaagaacatcttgataaacacaggtctattgagcttgaaacgttgcttggtagaaagctaatagcatacaaatttacagacgcagttaactatccaattcaaggctcaggcagtgatttattaaaaatggctgttgtgtttttctttaaagaaagaaacgatttagatgcgaaggttgttaaccttgttcatgatgaaatcctggtagaagttgctgcaaaagatatagaaaaggcaaaagaggttttatcttcatctatggaaaaagcaggaaaattaattttaaaagatgttccagttgcttttgaaatggttgtttctgatagttggaataaggggtaa

SEQ ID NO: 51 - protein, 587 aa, Sulfurihydrogenibium sp.
MMDINYITQENQLESLKVLQDTPYLYLDTEVMIKDFENIDFFNDKIRLIQIGDEENTFVIDLLKINPEVVKNHIQNLIENKGIIGHNLKFDLKFLKTNLNILPKIVFDTMIASQILAKGDSNQRHSLSASAKRFVSLDVDKTYQKSPWWAKDLSSEQIEYAAKDIDTLRHLFKEEKNQLNQDNLHKKASGETFKVFGVINPVAALEMAFLPALVEIELSGIPIDEEEAKKLLKQKESEFQSDYMKFKIKTGADPFSPQQVVNYLTNKLKIKLPKTEKGSFSSQDVFLKDYEDIEEVRLLLKLRADKKIIDKIKEILQFTRNERVYGEFKQIGAATGRMSSLRPNLQNIPKNLKYLFKPKEGYKFIVADYSQIELRIAAQYTKDENMITAFNEGKDLHKLTASIITGKSYDEITKEERQLAKAINFGLIYGMSPKSLVEYAKANYGVSISLQEAKKFHENYFKFYKSFKDWHDKVKEHLDKHRSIELETLLGRKLIAYKFTDAVNYPIQGSGSDLLKMAVVFFFKERNDLDAKVVNLVHDEILVEVAAKDIEKAKEVLSSSMEKAGKLILKDVPVAFEMVVSDSWNKG

SEQ ID NO: 52 - DNA, 2568 bp, Dictyoglomus turgidum
atggaacaaaaaaccctttgggatcttttccaagaaaatacagaaagagcccttaagaagattttagttattgatggttccagcatcatatatagggtttattatgcccttcccccattaaagacaaaaaatggggagctgaccaatgccctttatggttttataaggatacttttaaaggctgtagaggattttaaacctgatcttttaggtattgccttcgacagacctgagccaacctttaggcatgttatctataaagaatataaagcaaaaagaccaccaatgaaagatgatttgaaagctcaaattccttggataagggaatttttgaggttaaatgatatacctatattggaggagcctgggtatgaagctgatgatatcattgctactataataaaaaggtataaggacgatttaaaatatattctttccggagatttagatcttttacaattagtctctgataaaacttttttaatacatcctcaaagaggaatcacagagtttacgatttatgatccaaaggctgttaaagaaagatttggtgtagagcctaaaaaaattcctctttataaggtccttgtgggagatgagtcggataatattcctggaataaacggaataggacctaagaaagcatcaaaaattcttgaaaagatctcaactttagaagagtttaaagataaagtgaggttcttagagagcgatttaagagagattattgagaaaaactgggatattattgagagaaatttagagcttgttacattaaaaaatatagataaagattttgttcttaaaccttttgaaataaaaaaggatgaaaaactcatagaatttttgaaaagatatgaattaaaaagcattcttcaaaaactttttcctgatcttgaagaaagggaaaatatagaaattaaagatgtaaaggaaatcaattttgaagaggcaaaaaaggaaggttgttttgcttttaaatgccttggagaaaaaggctttgaaggaatatccatctcctttaaggaaggagaaggatattttatagcttcctttgactttaatgatgaagttaaagggaaagttaaagatattatttctttcgaaaatattaaaaagattggagcttatatacagagggatctacattttctggactgtaaaataaaaggggaggtgtttgatgttagtctcgcatcctatcttttaaatccagaaagacaaaatcattcccttgacatacttataagagagtatttaaataggacctcttttattcctcaaaagtatgctgcttatctctttcctttaaaaactattctagaagaaaggataaaaaaggaagaattggaatttgtgctttttaatatagaaacaccgcttattcctgtactttactccatggaaaaatggggaataaaggtagataaggagtatttaaaaagtctctctgatgaattttgtgagagaattaagaaattggaagaggaaatatatgaacttgcaggtatgaagtttaatcttaattctccaaaacaactttctgaggttttatttgagagattgaagcttccttctggcaagaaaggaaaaacaggatattctacatcatctttggtgcttcaaaatttactgaatgctcatcctattgtgataaaaatcctccaatatagggagttatataaacttaaaagcacctatatagatgctattcctaatcttataaattcacaaacaggcagggttcatactaaatttaaccccacaggtacagccacaggaaggataagtagtagtgaacccaatctacaaaatattcccataaaaagcgaggaaggaagaaagataaggagagcctttatagcagatgatggatattattttgtatctcttgattattcccaaatagagcttagaattatggctcacctctctcaagaacctaaattaatatcagccttccaaaagggtgaagatattcatagaagaacagcagcagaaattttcggagtgcctgaagatgaagtagatgatcttttgaggtcgagggcaaaggcggttaactttggaattatttatggcatctcttcctttgggctttctgaaactgcaagtatcactccggaagaggctgaaaaatttatagattcatattttaaacattatccaagggtaaagctctttatagataaaactatttatgaggcaagagaaaagttatatgtaaagactttatttggaagaaaaagatatatacctgaaattagaagtataaataagcaggtgaggaatgcttatgaaaggatagctataaatgcgcctattcaaggaacagcggcggatataataaaacttgccatgatagagatttataaagaaatagaggaaaaaaatcttaagtcaagaatacttttacagattcacgatgaacttattcttgaagtgcctgaagaagaaatggagtttacccctttgatggcaaaggaaaagatggaaaaggttgtagaactttctgttcctcttgtggttgagatttcagtgggtaaaaatctggctgagctgaaatga

SEQ ID NO: 53 - protein, 855 aa, Dictyoglomus turgidum
MEQKTLWDLFQENTERALKKILVIDGSSIIYRVYYALPPLKTKNGELTNALYGFIRILLKAVEDFKPDLLGIAFDRPEPTFRHVIYKEYKAKRPPMKDDLKAQIPWIREFLRLNDIPILEEPGYEADDIIATIIKRYKDDLKYILSGDLDLLQLVSDKTFLIHPQRGITEFTIYDPKAVKERFGVEPKKIPLYKVLVGDESDNIPGINGIGPKKASKILEKISTLEEFKDKVRFLESDLREIIEKNWDIIERNLELVTLKNIDKDFVLKPFEIKKDEKLIEFLKRYELKSILQKLFPDLEERENIEIKDVKEINFEEAKKEGCFAFKCLGEKGFEGISISFKEGEGYFIASFDFNDEVKGKVKDIISFENIKKIGAYIQRDLHFLDCKIKGEVFDVSLASYLLNPERQNHSLDILIREYLNRTSFIPQKYAAYLFPLKTILEERIKKEELEFVLFNIETPLIPVLYSMEKWGIKVDKEYLKSLSDEFCERIKKLEEEIYELAGMKFNLNSPKQLSEVLFERLKLPSGKKGKTGYSTSSLVLQNLLNAHPIVIKILQYRELYKLKSTYIDAIPNLINSQTGRVHTKFNPTGTATGRISSSEPNLQNIPIKSEEGRKIRRAFIADDGYYFVSLDYSQIELRIMAHLSQEPKLISAFQKGEDIHRRTAAEIFGVPEDEVDDLLRSRAKAVNFGIIYGISSFGLSETASITPEEAEKFIDSYFKHYPRVKLFIDKTIYEAREKLYVKTLFGRKRYIPEIRSINKQVRNAYERIAINAPIQGTAADIIKLAMIEIYKEIEEKNLKSRILLQIHDELILEVPEEEMEFTPLMAKEKMEKVVELSVPLVVEISVGKNLAELK

SEQ ID NO: 54 - DNA, 2571 bp, Dictyoglomus thermophilum
atggagcagaaatctctgtgggatctttttcaagaaaataccgagaaagagtccaaaaggaagattctgattattgatggctcaagcctcatatacagggtttattacgcccttccccctttaaagacaaaaaatggtgaattaactaatgctctttatggcttcataagaatacttttaaaggccgtagaagattttaatcctgatcttgtaggcgttgcctttgatagacctgaacctacttttaggcatgtgatttataaagagtataaggctaagagaccacctatgaaggatgatttgaaagcgcagataccatggataagagaatttctaaggttaaatgatatacctctattggaagagcctggctatgaagcggatgatataatagctactatagtgaataaatataaggatgatttaaaatatattctctctggagatttagatcttttgcaattagtctcggacaaaacctttctaatacatcctcaaaagggaattactgagtttactatttatgatccaaaagctgtaaaggataggtttggagtagagccctataagattcccttatacaaagtattagtaggggacgaatctgataatattccaggagtaaatggaataggtcctaaaaaggcctcaaagattcttgagaaaatttcaagtgtagatgaatttaaaagtaaaataaaagttttggatagtgatttaagggagcttattgagaaaaattggaatattattgaaagaaatttagaacttgttactttaaaaaatatagataaggatcttattcttaaacccttcgagattaaaagagatgaaaaagtaatagattttttgaagagatatgaacttaagagtattcttcaaaagttatttcctgatcttcaagaggaagaaaatatagagattaaagatgtcgaagagatcaattttaatgaggtagaaaaagaaggctactttgcctttaaatgtcttggagatagggcttttgagggtatttctctttccttcaaggagggggaaggatattttatatctccttttgatttcaataatgagataagaaagaagattgaaaatataatttcttcagagaatgttaaaaaaattggctcttatattcaaagagatttacattttttaaactgtaaaataaagggcgatgtatttgatgttagtctcgcatcttatcttttgaaccctgaaagacaaaatcactctcttgatattttgataggagagtatctaaataaaacctcttttattcctcaaaaatacgctggttatctttttccgttaaagtctattcttgaggagaggataaagaatgaagggttagaatttgtactttataacatagagattccattaatccctgtactttactccatggagaagtgggggataaaggtagataaggaatatttaaaacagctttctgatgaattctgcgagagaattaaaaaattggaagaagagatatatgaacttgcaggaaccagatttaatctcaattctccaaaacaactttctgaagttttatttgagaggttaaaacttccttctggtaagaaaggaaaaacaggatattctacgtcgtcttctgtgcttcaaaacttaataaatgctcatcctatagtgagaaaaatcctccaatatagagaactctataaattgaagagtacttatgtggatgctattcctaatctggttaatccacaaacaggtagagttcatacaaaatttaatcctacaggtacagctacaggaagaataagtagtagtgaacctaatcttcagaatattcctataaaaagtgaagaaggtagaaagataagaagagccttcgtgtcagaagatggatattttcttgtatctcttgattattctcagatagagctaaggattatggctcatctttctcaggagcctaaattaatatctgccttccaaaaaggagaggatattcatagaagaacagcatcggagatttttggagtgccagaggaagaagttgatgatcttttaaggtcaagggcaaaggccgttaattttggaattatttatggtatctcttcttttggactttctgagactgtaagtattacaccagaagaggcagagaaatttatagactcgtattttaagcactatccaagagtgaagctttttatagataagactattcatgaggcaagagaaaaactgtacgttaaaaccttatttggcagaaaaagatatattcctgagattaagagcataaataaacaggtaaggaatgcctatgaaaggatagcaataaatgcgccaattcagggaacagctgctgatattataaaacttgccatgatagaaatttacaaggagattgaaaataaaaatctcaagtcaagaatactccttcaaattcatgatgagcttattcttgaagtgccagaggaggagatggaatttactcctttaatggcaaaggaaaaaatggaaaaggtggtagaactttcggttcctcttgtagttgaaatctcggtaggtaaaaatcttgctgaattaaaatga

SEQ ID NO: 55 - protein, 856 aa, Dictyoglomus thermophilum
MEQKSLWDLFQENTEKESKRKILIIDGSSLIYRVYYALPPLKTKNGELTNALYGFIRILLKAVEDFNPDLVGVAFDRPEPTFRHVIYKEYKAKRPPMKDDLKAQIPWIREFLRLNDIPLLEEPGYEADDIIATIVNKYKDDLKYILSGDLDLLQLVSDKTFLIHPQKGITEFTIYDPKAVKDRFGVEPYKIPLYKVLVGDESDNIPGVNGIGPKKASKILEKISSVDEFKSKIKVLDSDLRELIEKNWNIIERNLELVTLKNIDKDLILKPFEIKRDEKVIDFLKRYELKSILQKLFPDLQEEENIEIKDVEEINFNEVEKEGYFAFKCLGDRAFEGISLSFKEGEGYFISPFDFNNEIRKKIENIISSENVKKIGSYIQRDLHFLNCKIKGDVFDVSLASYLLNPERQNHSLDILIGEYLNKTSFIPQKYAGYLFPLKSILEERIKNEGLEFVLYNIEIPLIPVLYSMEKWGIKVDKEYLKQLSDEFCERIKKLEEEIYELAGTRFNLNSPKQLSEVLFERLKLPSGKKGKTGYSTSSSVLQNLINAHPIVRKILQYRELYKLKSTYVDAIPNLVNPQTGRVHTKFNPTGTATGRISSSEPNLQNIPIKSEEGRKIRRAFVSEDGYFLVSLDYSQIELRIMAHLSQEPKLISAFQKGEDIHRRTASEIFGVPEEEVDDLLRSRAKAVNFGIIYGISSFGLSETVSITPEEAEKFIDSYFKHYPRVKLFIDKTIHEAREKLYVKTLFGRKRYIPEIKSINKQVRNAYERIAINAPIQGTAADIIKLAMIEIYKEIENKNLKSRILLQIHDELILEVPEEEMEFTPLMAKEKMEKVVELSVPLVVEISVGKNLAELK

SEQ ID NO: 56 - DNA, 756 bp, Sulfolobus virus 1
atggtaaggaacatgaagatgaagaagagtaatgaatggttatggttagggactaaaattataaacgcccataagactaacggctttgaaagtgcgattattttcgggaaacaaggtacgggaaagactacttacgcccttaaggtggcaaaagaagtttaccagagattaggacatgaaccggacaaggcatgggaactggcccttgactctttattctttgagcttaaagatgcattgaggataatgaaaatattcaggcaaaatgataggacaataccaataataattttcgacgatgctgggatatggcttcaaaaatatttatggtataaggaagagatgataaagttttaccgtatatataacattattaggaatatagtaagcggggtgatcttcactaccccttcccctaacgatatagcgttttatgtgagggaaaaggggtggaagctgataatgataacgagaaacggaagacaacctgacggtacgccaaaggcagtagctaaaatagcggtgaataagataacgattataaaaggaaaaataacaaataagatgaaatggaggacagtagacgattatacggtcaagcttccggattgggtatataaagaatatgtggaaagaagaaaggtttatgaggaaaaattgttggaggagttggatgaggttttagatagtgataacaaaacggaaaacccgtcaaacccatcactactaacgaaaattgacgacgtaacaagatag

SEQ ID NO: 57 - protein, 251 aa, Sulfolobus virus 1
MVRNMKMKKSNEWLWLGTKIINAHKTNGFESAIIFGKQGTGKTTYALKVAKEVYQRLGHEPDKAWELALDSLFFELKDALRIMKIFRQNDRTIPIIIFDDAGIWLQKYLWYKEEMIKFYRIYNIIRNIVSGVIFTTPSPNDIAFYVREKGWKLIMITRNGRQPDGTPKAVAKIAVNKITIIKGKITNKMKWRTVDDYTVKLPDWVYKEYVERRKVYEEKLLEELDEVLDSDNKTENPSNPSLLTKIDDVTR

SEQ ID NO: 58 - DNA, 24 bp, Artificial Sequence
  Synthetic oligonucleotide
gaagaggtggcgcgtaacgcgtcc

SEQ ID NO: 59 - DNA, 24 bp, Artificial Sequence
  Synthetic oligonucleotide
aacgaagtccgtgaagacggaaac

SEQ ID NO: 60 - DNA, 2772 bp, Artificial Sequence
  74-like family consensus
atggtgtatttgaaccaatawgatgtgcctgttgttatcgctggagagcctcagaacttacctgttgaggcaatgcttcaggaaaacgaaggtctcttcagaaggtctttagttatatctcttgacgacaggaatttgaaaaagtatgaaaagctaatgagcttttacaacgagctgatagaacaattttacaaccatcatggtttcgcttataagataattgaagacttagaaaggatagacaaacgcatcattgaggagcaagcacgtaagttcgcaagtattaagttgcttcaggacacactgataagagaaggttataaagaccaaaaggtaaagcttctttctgctattgacaacttactttcaagaatacttttcgctcttagaattttcactgatgttcttcaaattccgcaagaagagtgggaagagcttttgacacaaatagctttatacgtggatacaagcttatcaacattctataaactattcctgccacgtgagaaaaggcttgaagaagagcttgtagattttctgacacaattgactgatattctttacaagactatcaacgatagaaacagaccagacttgcctcggactttgggaggagcttctttggataagcttatcaaaatagcaaaggtgcaaataccaagctcgcaagtcttgaagtattctaaagatgttcttctcaagaaatacaaaacagcaagagcttatttattcgcaagctctgtgcttgttagcagaattgatgatgcagaattgataaacaacgacgtggagagattgttagaaaatgtaaataacatgcctgacaaagatagagacctttgcctttatgcttatatcaatactgcaaaacgtgttttgagtgagaaagtgtttaactctgtttataccgtattgaaaagcaaaggtgtagatgtggataagtatttgaatatgagctttgtagatgacgacgatgacacagaaccaccaccaattggcggtaataataccgaaccaccgaataacaatggtggtgctgataacgaaccgcaagaagataatcgttttgctgaaattaaagaagtcaaacaactaaccgcacctctgcctgatgtagagcttataacagactttaagcaaattaaagaccacatacaatacgatggaactatatacatagacgtagaagcagatgttgaaacgcaacaacctattttgctcgctctctatcaaaaacactggaaaaaggtttatgcggtagatttacgtaaagtcaagctggagcaagtcaaagaatggcttctcagatttaacgtaataagtggctgggggcttaactatgaccttgtcaggcttggtttctcttatgaagagttaaaagaccatgtcgtgttagacttgcttttacttgcacgggaaaagctttacaaaagagacagctttaagttagatgatgtgttaaaagatgtgcttggagtggagtatccatttgacaaaacgaaaatcagaaaaacattcaaaaacacactttactttacacaagaacagctacaatatgttggtcttgatgtttattatttgcctaaactgtttgatgccatttctgatgacagtttaagcatagttcaacagttagaccaagaagctttaaaggtttgtgtggatacaagccaaagaggaatgccgttcttggttgaagaagcgagagcaaagctaattgttctacgacaagaacttgatgttataacyaaagagcttggctttaatccacgctccccgcaacaaactaaaaacgctttatcggttcaagatacgagggaagagacgcttcaagacctaataatcaacaatggagtaagraaggaaatagcggagaaggttttacttgcaagaaagatagctaaagaaatttctatgcttgagacgtatatacagcacggagttagagtgaaaggcatcttttggacaacacaggctccaagcggacgtatgagttgcaatgatgaaaatcttcagcaggttccaagaagtttgagagacttgtttggctttactgaagataacgataaagtccttataacagctgactttccgcagatagaattgagactggcaggtgctttatggcgtgagccgaaatttgtagaagcgtttagaaaaggtgaagaccttcataaaataacggcttcaattatctatggcgtgcctgtagatgaagtatctaaggagcaaaggcaggtggcaaagtctgccaattttggactaatttacggagcttctccacaaggtttccaaagatattgcataagcaatggaattccgatggatttagagacggcacaactaattcacacgaaattctttgagacttatacaaagatagctaaagaacatgagcttgtcaaagattactttagatacaacacagaagctgaaggtgaaacttggcttggtagaaaatacgtagctaaatctccacagcaaatgcttaactatcaaatacaaggttcaggtgcagagttgtttaagaagacgatagtagagcttaagaagaagtatccaagccttgctattgttaatttggtgcatgacgagatagtgattgaagcagacagacaaacagcagaggatatagctttaattgtaaaagcggagatggaacaagcttgggagtggtgtttagaagaagctaaacagcaaggtaggttgattgaagaatttaaacttgaagttgaaatgcctaacattagcaaaaaatgggaaaaaccataa

SEQ ID NO: 61 - protein, 923 aa, Artificial Sequence
  74-like family consensus
MVYLNQXDVPVVIAGEPQNLPVEAMLQENEGLFRRSLVISLDDRNLKKYEKLMSFYNELIEQFYNHHGFAYKIIEDLERIDKRIIEEQARKFASIKLLQDTLIREGYKDQKVKLLSAIDNLLSRILFALRIFTDVLQIPQEEWEELLTQIALYVDTSLSTFYKLFLPREKRLEEELVDFLTQLTDILYKTINDRNRPDLPRTLGGASLDKLIKIAKVQIPSSQVLKYSKDVLLKKYKTARAYLFASSVLVSRIDDAELINNDVERLLENVNNMPDKDRDLCLYAYINTAKRVLSEKVFNSVYTVLKSKGVDVDKYLNMSFVDDDDDTEPPPIGGNNTEPPNNNGGADNEPQEDNRFAEIKEVKQLTAPLPDVELITDFKQIKDHIQYDGTIYIDVEADVETQQPILLALYQKHWKKVYAVDLRKVKLEQVKEWLLRFNVISGWGLNYDLVRLGFSYEELKDHVVLDLLLLAREKLYKRDSFKLDDVLKDVLGVEYPFDKTKIRKTFKNTLYFTQEQLQYVGLDVYYLPKLFDAISDDSLSIVQQLDQEALKVCVDTSQRGMPFLVEEARAKLIVLRQELDVIXKELGFNPRSPQQTKNALSVQDTREETLQDLIINNGVXKEIAEKVLLARKIAKEISMLETYIQHGVRVKGIFWTTQAPSGRMSCNDENLQQVPRSLRDLFGFTEDNDKVLITADFPQIELRLAGALWREPKFVEAFRKGEDLHKITASIIYGVPVDEVSKEQRQVAKSANFGLIYGASPQGFQRYCISNGIPMDLETAQLIHTKFFETYTKIAKEHELVKDYFRYNTEAEGETWLGRKYVAKSPQQMLNYQIQGSGAELFKKTIVELKKKYPSLAIVNLVHDEIVIEADRQTAEDIALIVKAEMEQAWEWCLEEAKQQGRLIEEFKLEVEMPNISKKWEKP

SEQ ID NO: 62 - DNA, 1617 bp, Artificial Sequence
  Truncated 74-like family
caatacgatggaactatatacatagacgtagaagcagatgttgaaacgcaacaacctattttgctcgctctctatcaaaaacactggaaaaaggtttatgcggtagatttacgtaaagtcaagctggagcaagtcaaagaatggcttctcagatttaacgtaataagtggctgggggcttaactatgaccttgtcaggcttggtttctcttatgaagagttaaaagaccatgtcgtgttagacttgcttttacttgcacgggaaaagctttacaaaagagacagctttaagttagatgatgtgttaaaagatgtgcttggagtggagtatccatttgacaaaacgaaaatcagaaaaacattcaaaaacacactttactttacacaagaacagctacaatatgttggtcttgatgtttattatttgcctaaactgtttgatgccatttctgatgacagtttaagcatagttcaacagttagaccaagaagctttaaaggtttgtgtggatacaagccaaagaggaatgccgttcttggttgaagaagcgagagcaaagctaattgttctacgacaagaacttgatgttataacyaaagagcttggctttaatccacgctccccgcaacaaactaaaaacgctttatcggttcaagatacgagggaagagacgcttcaagacctaataatcaacaatggagtaagraaggaaatagcggagaaggttttacttgcaagaaagatagctaaagaaatttctatgcttgagacgtatatacagcacggagttagagtgaaaggcatcttttggacaacacaggctccaagcggacgtatgagttgcaatgatgaaaatcttcagcaggttccaagaagtttgagagacttgtttggctttactgaagataacgataaagtccttataacagctgactttccgcagatagaattgagactggcaggtgctttatggcgtgagccgaaatttgtagaagcgtttagaaaaggtgaagaccttcataaaataacggcttcaattatctatggcgtgcctgtagatgaagtatctaaggagcaaaggcaggtggcaaagtctgccaattttggactaatttacggagcttctccacaaggtttccaaagatattgcataagcaatggaattccgatggatttagagacggcacaactaattcacacgaaattctttgagacttatacaaagatagctaaagaacatgagcttgtcaaagattactttagatacaacacagaagctgaaggtgaaacttggcttggtagaaaatacgtagctaaatctccacagcaaatgcttaactatcaaatacaaggttcaggtgcagagttgtttaagaagacgatagtagagcttaagaagaagtatccaagccttgctattgttaatttggtgcatgacgagatagtgattgaagcagacagacaaacagcagaggatatagctttaattgtaaaagcggagatggaacaagcttgggagtggtgtttagaagaagctaaacagcaaggtaggttgattgaagaatttaaacttgaagttgaaatgcctaacattagcaaaaaatgggaaaaaccataa

SEQ ID NO: 63 - protein, 538 aa, Artificial Sequence
  Truncated 74-like family
QYDGTIYIDVEADVETQQPILLALYQKHWKKVYAVDLRKVKLEQVKEWLLRFNVISGWGLNYDLVRLGFSYEELKDHVVLDLLLLAREKLYKRDSFKLDDVLKDVLGVEYPFDKTKIRKTFKNTLYFTQEQLQYVGLDVYYLPKLFDAISDDSLSIVQQLDQEALKVCVDTSQRGMPFLVEEARAKLIVLRQELDVIXKELGFNPRSPQQTKNALSVQDTREETLQDLIINNGVXKEIAEKVLLARKIAKEISMLETYIQHGVRVKGIFWTTQAPSGRMSCNDENLQQVPRSLRDLFGFTEDNDKVLITADFPQIELRLAGALWREPKFVEAFRKGEDLHKITASIIYGVPVDEVSKEQRQVAKSANFGLIYGASPQGFQRYCISNGIPMDLETAQLIHTKFFETYTKIAKEHELVKDYFRYNTEAEGETWLGRKYVAKSPQQMLNYQIQGSGAELFKKTIVELKKKYPSLAIVNLVHDEIVIEADRQTAEDIALIVKAEMEQAWEWCLEEAKQQGRLIEEFKLEVEMPNISKKWEKP

SEQ ID NO: 64 - DNA, 1877 bp, Unknown
  Clone 3173V6 from uncultured newly isolated virus
atgatgacgatgatacaccaccaaacaataatttaccaccagtagaagagtttgattatgaaaatgaaggagacgaagacaaagaggaagaagatgagttagaaaaacactttacaggagaagatgggctatctctacctaagatgatgaacacaccaaaaccaattcttaaacctcaatcaaaagctttggtagaaccagagctttgcaatagtattgatgaaataccagcgaaatataatgaaccgatatactttgacttagaaactgacgaggatagaccagtgcttgcaagtatttatcaacctcactttgaacgcaaggtgtattgtctcaatcttcttagagaaaagctagcaaggttcaaagagtggcttctaaagttttcagaaatacgaggctggggtcttgattttgatttaagggttcttggctacacatatgaacagcttagaaacaaaaagattatagatgttcagcttgcgttaaaggtccagcactacgagagatttaagcaaggtggaaccaaaggcgaaggttttaggcttgatgatgtggcacgagatttgcttggtatagaatatccgatgaacaaaacaaaaattcgtgaaaccttcaaaaacaacatgtttcattcatttagcaacgaacaacttctttatgcctcgtttgatgcatacataccacacttgctttatgaacagctaacatcaagcacacttaacagtcttgtttatcaacttgaccaacaagcacaaaagattgtaatagaaacatcacaacacggtatgccagtgaaactgaaagcattagaggaagaaatacatagactaactcaactgcgtagtgaaatgcaaaggcagataccgtttaattacaattctccgaagcaaacagcgaagttttttggagtagatagttcatcaaaagatgtattgatggacttggctttgcagggtaatgaaatggctaagaaagttctggaagcaagacaaatagaaaagtctttaacctttgctaaagaactttatgaccttgcaaaaaagaacggcagaatatatggcaatttctttaccaccaccgcaccatcaggtagaatgtcttgttccgatataaatctacaacagataccacgcagacttagaccattcataggctttgaaacagaagacaaaaagcttatcaccgcagactttcctcaaattgaactcagacttgcaggtgtgatttgggatgaacctaaattcatagaagcatttaggcaaggcatagaccttcacaaacttaccgcatcaatactgtttgataagaacatagaagaagtaagcaaagaagagagacaaatcggaaaatcagcgaactttggacttatctacggcattgcaccaaaaggtttcgcagaatattgtataacgaacggtattaatatgacagaagaacaagcatacgagatagttaaaaagtggaagaggtattacaccaagattacggaacagcatcaagtagcatatgaacgatttaaatacaacgagtatgtggataacgaaacgtggttggctagaacatatcgtgcatacaaacctcaagatttgcttaactatcaaatacaaggcagtggtgcagaactattcaaaaaagctatagtattactcaaggaagcaaagccagatttgaagatagtcaatctcgtacatgatgagatagtagtggaagctgacagtaaagaagcacaagacttggctaagctaattaaagaaaagatggaagaagcgtgggattggtgtcttgaaaaagcagaagagtttgggtatagaattgctaaaataaaacttgaagtggaggagccaaatgttggcgacacatgggagaaaccttga

SEQ ID NO: 65 - DNA, 1877 bp, Unknown
  Clone 3173V7 from uncultured newly isolated virus
atgatgacgatgatacaccaccaaacaataatttaccaccagtagaagagtttgattatgaaaatgaaggagacgaagacaaagaggaagaagatgagttagaaaaacactttacaggagaagatgggctatctctacctaagatgatgaacacaccaaaaccaattcttaaacctcaatcaaaagctttggtagaaccagtgctttgcaatagtattgatgaaataccagcgaaatataatgaaccgatatactttgacttagaaactgacgaggatagaccagtgcttgcaagtatttatcaacctcactttgaacgcaaggtgtactgtctcaatcttcttagagaaaagctagcaaggttcaaagagtggcttctaaagttttcagaaatacgaggctggggtcttgattttgatttaagggttcttggctacacatatgaacagcttagaaacaaaaagattatagatgttcagcttgcgttaaaggtccagcactacgagagatttaagcaaggtggaaccaaaggcgaaggttttaggcttgatgatgtggcacgagatttgcttggtatagaatatccgatgaacaaaacaaaaattcgtgaaaccttcaaaaacaacatgtttcattcatttagcaacgaacaacttctttatgcctcgtttgatgcatacataccacacttgctttatgaacagctaacatcaagcacacttaacagtcttgtttatcaacttgaccaacaagcacaaaagattgtaatagaaacatcacaacacggtatgccagtgaaactgaaagcattagaggaagaaatacatagactaactcaactgcgtagtggaatgcaaaggcagataccgtttaattacaattctccgaagcaaacagcgaagttttttggagtagatagttcatcaaaagatgtattgatggacttggctttgcagggtaatgaaatggctaagaaagttctggaagcaagacaaatagaaaagtctttaacctttgctaaagaactttatgaccttgcaaaaaagaacggcagaatatatggcaatttctttaccaccaccgcaccatcaggtagaatgtcttgttccgatataaatctacaacagataccacgcagacttagaccattcataggctttgaaacagaagacaaaaagcttatcaccgcagactttcctcaaattgaactcagacttgcaggtgtgatttgggatgaacctaaattcatagaagcatttaggcaaggcatagaccttcacaaacttaccgcatcaatactgtttgataagaacatagaagaagtaagcaaagaagagagacaaatcggaaaatcagcgaaccttggacttatctacggcattgcaccaaaaggtttcgcagaatattgtataacgaacggtattaatatgacagaagaacaagcatacgagatagttaaaaagtggaagaggtattacaccaagattacggaacagcatcaagtagcatatgaacgatttaaatacaacgagtatgtggataacgaaacgtggttggctagaacatatcgtgcatacaaacctcaagatttgcttaactatcaaatacaaggcagtggtgcagaactattcaaaaaagctatagtattactcaaggaagcaaagccagatttgaagatagtcaatctcgtacatgatgagatagtagtggaagctgacagtaaagaagcacaagacttggctaagctaattaaagaaaagatggaagaagcgtgggattggtgtcttgaaaaagcagaagagtttgggtatagaattgctaaaataaaacttgaagtggaggagccaaacgttggcgacacatgggagaaaccttga

SEQ ID NO: 66 - DNA, 1877 bp, Unknown; identical to SEQ ID NO: 65
  Clone 3173V8 from uncultured newly isolated virus

SEQ ID NO: 67 - protein, 577 aa, Unknown
  Clone 3173V1 from uncultured newly isolated virus
MMNTPKPILKPQSKALVEPVLCNSIDEIPAKYNEPIYFDLETDEDRPVLASIYQPHFERKVYCLNLLRERLARFKEWLLKFSEIRGWGLDFDLRVLGYTYEQLRNKKIIDVQLALKVQHYERFKQGGAKGEGFRLDDVARDLLGIEYPMNKTKIRETFKNNMFHSFSNEQLLYASFDAYIPHLLYEQLTSSTLNSLVYQLDQQAQKIVIETSQHGMPVKLKALEEEIHRLTQLRSEMQRQIPFNYNSPKQTAKFFGVDSSSKDVLMDLALQGNEMAKKVLEARQIEKSLTFAKELYDLAKKNGRIYGNFFATTAPSGRMSCSDINLQQIPRRLRPFIGFETEDKKLITADFPQIELRLAGVIWDEPKFIEAFRQGIDLHKLTASILFDKNIEEVSKEERQIGKSANFGLIYGIAPKGFAEYCITNGINMTEEQAYEIVKKWKRYYTKITEQHQVAYERFKYNEYVDNETWLARTYRAYKPQDLLNYRIQGSGAELFKKAIVLLKEAKPDLKIVNLVHDEIVVEADSKEAQDLAKLIKEKMEEAWDWCLEKAEEFGYRIAKIKLEVEEPNVGDTWEKP

SEQ ID NO: 68 - protein, 577 aa, Unknown
  Clone 3173V2 from uncultured newly isolated virus
MMNTPKPILKPQSKALVEPVLCNSIDEIPAKYNEPIYFDLETDEDRPVLASIYQPHFERKVYRLNLLREKLARFKEWLLKFSEIRGWGLDFDLRVLGYTYEQLRNKKIIDVQLALKVQHYERFKQGGTKGEGFRLDDVARDLLGIEYPMNKTKIRETFKNNMFHSFSNEQLLYASFDAYIPHLLYEQLTSSTLNSLVYQLDQQAQKIVIETSQHGMPVKLKALEEEIHRLTQLRSEMQRQIPFNYNSPKQTAKFFGVDSSSKDVLMDLALQGNEMAKKVLEARQIEKSLTFAKELYDLAKRNGRIYGNFFTTTAPSGRMSCSDINLQQIPRRLRPFIGFETEDKKLITADFPQIELRLAGVIWDEPKFIEAFRQGIDLHKLTASILFDKNIEEVSKEERQIGKSANFGLIYGIAPKGFAEYCITNGINMTEEQAYEIVKKWKRYYTKITEQHQVAYERFKYNEYVDNETWLARTYRAYKPQDLLNYQIQGSGAELFKKAIVLLKEAKPDLKIVNLVHDEIVVEADSKEAQDLAKLIKEKMEEAWDWCLEKAEEFGYRIAKIKLEVEEPNVGDTWEKP

SEQ ID NO: 69 - protein, 577 aa, Unknown; identical to SEQ ID NO: 68
  Clone 3173V3 from uncultured newly isolated virus

SEQ ID NO: 70 - protein, 577 aa, Unknown
  Clone 3173V4 from uncultured newly isolated virus
MMNTPKPILKPQSKALVEPVLCNSIDEIPAKYNEPIYFDLETDEDRPVLASIYQPHFERKVYCLNLLREKLARFKEWLLKFSEIRGWGLDFDLRVLGYTYEQLRNKKIIDVQLALKVQHYERFKQGGTKGEGFRLDDVARDLLGIEYPMNKTKIRETFKNNMFHSFSNEQLLYASFDAYIPHLLYEQLTSSTLNSLVYQLDQQAQKIVIETSQHGMPVKLKALEEEIHRLTQLRSEMQRQIPFNYNSPKQTAKFFGVDSSSKDVLMDLALQGNEMAKKVLEARQIEKSLTFAKELYDLAKKNGRIYGNFFTTTAPSGRMSCSDINLQQIPRRLRPFIGFETEDKKLITADFPQIELRLAGVIWDEPKFIEAFRQGIDLHKLTASILFGKNIEEVSKEERQIGKSANFGLIYGIAPKGFAEYCITNGINMTEEQAYEIVKKWKRYYTKITEQHQVAYERFKYNEYVDNETWLARTYRAYKPQDLLNYQIQGSGAELFKKAIVLLKEAKPDLKIVNLVHDEIVVEADSKEAQDLAKLIKEKMEEAWDWCLEKAEEFGYRIAKIKLEVEEPNVGDTWEKP

SEQ ID NO: 71 - protein, 577 aa, Unknown; identical to SEQ ID NO: 70
  Clone 3173V5 from unknown newly isolated virus

SEQ ID NO: 72 - protein, 577 aa, Unknown
  Clone 3173V6 from uncultured newly isolated virus
MMNTPKPILKPQSKALVEPELCNSIDEIPAKYNEPIYFDLETDEDRPVLASIYQPHFERKVYCLNLLREKLARFKEWLLKFSEIRGWGLDFDLRVLGYTYEQLRNKKIIDVQLALKVQHYERFKQGGTKGEGFRLDDVARDLLGIEYPMNKTKIRETFKNNMFHSFSNEQLLYASFDAYIPHLLYEQLTSSTLNSLVYQLDQQAQKIVIETSQHGMPVKLKALEEEIHRLTQLRSEMQRQIPFNYNSPKQTAKFFGVDSSSKDVLMDLALQGNEMAKKVLEARQIEKSLTFAKELYDLAKKNGRIYGNFFTTTAPSGRMSCSDINLQQIPRRLRPFIGFETEDKKLITADFPQIELRLAGVIWDEPKFIEAFRQGIDLHKLTASILFDKNIEEVSKEERQIGKSANFGLIYGIAPKGFAEYCITNGINMTEEQAYEIVKKWKRYYTKITEQHQVAYERFKYNEYVDNETWLARTYRAYKPQDLLNYQIQGSGAELFKKAIVLLKEAKPDLKIVNLVHDEIVVEADSKEAQDLAKLIKEKMEEAWDWCLEKAEEFGYRIAKIKLEVEEPNVGDTWEKP

SEQ ID NO: 73 - protein, 577 aa, Unknown
  Clone 3173V7 from uncultured newly isolated virus
MMNTPKPILKPQSKALVEPVLCNSIDEIPAKYNEPIYFDLETDEDRPVLASIYQPHFERKVYCLNLLREKLARFKEWLLKFSEIRGWGLDFDLRVLGYTYEQLRNKKIIDVQLALKVQHYERFKQGGTKGEGFRLDDVARDLLGIEYPMNKTKIRETFKNNMFHSFSNEQLLYASFDAYIPHLLYEQLTSSTLNSLVYQLDQQAQKIVIETSQHGMPVKLKALEEEIHRLTQLRSEMQRQIPFNYNSPKQTAKFFGVDSSSKDVLMDLALQGNEMAKKVLEARQIEKSLTFAKELYDLAKKNGRIYGNFFTTTAPSGRMSCSDINLQQIPRRLRPFIGFETEDKKLITADFPQIELRLAGVIWDEPKFIEAFRQGIDLHKLTASILFDKNIEEVSKEERQIGKSANFGLTYGIAPKGFAEYCITNGINMTEEQAYEIVKKWKRYYTKITEQHQVAYERFKYNEYVDNETWLARTYRAYKPQDLLNYQIQGGGAELFKKAIILLKEAKPDLKIVNLVHDEIVVEADSKEAQDLAKLIKEKMEEAWDWCLEKAEEFGYRIAKIKLEVEEPNVGDTWEKP

SEQ ID NO: 74 - protein, 577 aa, Unknown
  Clone 3173V8 from uncultured newly isolated virus
MMNTPKPILKPQSKALVEPVLCNSIDEIPAKYNEPIYFDLETDEDRPVLASIYQPHFERKVYCLNLLREKLARFKEWLLKFSEIRGWGLDFDLRVLGYTYEQLRNKKIIDVQLALKVQHYERFKQGGTKGEGFRLDDVARDLLGIEYPMNKTKIRETFKNNMFHSFSNEQLLYASFDAYIPHLLYEQLTSSTLNSLVYQFDQQAQRIVIETSQHGMPVKLKALEEEIHRLTQLRSEMQRQIPFNYNSPKQTAKFFGVDSSSKDVLMDLALQGNEMAKKVLEARQIERSLTFAKELYDLAKKNGRIYGNFFTTTAPSGRMSCSDINLQQIPRRLRPFIGFETEDKKLITADFPQIELRLAGVIWDEPKFIEAFRQGIDLHKLTASILFDKNIEGVSKEERQIGKSANFGLIYGIAPKGFAEYCITNGINMTEEQAYEIVKKWKRYYTKITEQHQVAYERFKYNEYVDNETWLARTYRAYKPQDLLNYQIQGSGAELFKKAIVLLKEAKPDLKIVNLVHDEIVVEADSKEAQDLAKLIKEKMEEAWDWCLEKAEEFGYRIAKIKLEVEEPNVGDTWEKP

SEQ ID NO: 75 - protein, 577 aa, Unknown
  Clone 3173V9 from uncultured newly isolated virus
MMNTPKPILKPQSKALVEPVLCNSIDEIPAKYNEPIYFDLETDEDRPVLASIYQPHFERKVYCLNLLREKLARFKEWLLKFSEIRGWGLDFDLRVLGYTYEQLRNKKIIDVQLALKVQHYERFKQGGTKGEGFRLDDVARDLLGIEYPMNKTKIRETFKNNMFHSFSNEQLLYASFDAYIPHLLYEQLTSSTLNSLVYQLDQQAQKIVIETSQHGMPVKLKALEEEIHRLTQLRSGMQRQIPFNYNSPKQTAKFFGVDSSSKDVLMDLALQGNEMAKKVLEARQIEKSLTFAKELYDLAKKNGRIYGNFFTTTAPSGRMSCSDINLQQIPRRLRPFIGFETEDKKLITADFPQIELRLAGVIWDEPKFIEAFRQGIDLHKLTASILFDKNIEEVSKEERQIGKSANLGLIYGIAPKGFAEYCITNGINMTEEQAYEIVKKWKRYYTKITEQHQVAYERFKYNEYVDNETWLARTYRAYKPQDLLNYQIQGSGAELFKKAIVLLKEAKPDLKIVNLVHDEIVVEADSKEAQDLAKLIKEKMEEAWDWCLEKAEEFGYRIAKIKLEVEEPNVGDTWEKP

SEQ ID NO: 76 - protein, 577 aa, Unknown; identical to SEQ ID NO: 70
  Clone 3173V10 from uncultured newly isolated virus

SEQ ID NO: 77 - protein, 577 aa, Unknown; identical to SEQ ID NO: 73
  Clone 3173V11 from uncultured newly isolated virus

SEQ ID NO: 78 - protein, 14 aa, Escherichia coli
VSADYSQIELRIMA

SEQ ID NO: 79 - protein, 14 aa, Thermus aquaticus
VALDYSQIELRVLA

SEQ ID NO: 80 - protein, 14 aa, Artificial Sequence
  Consensus sequence
VXXDXSXIELRXLG

SEQ ID NO: 81 - protein, 14 aa, Artificial Sequence
  Consensus sequence
XXXDXPXIELRXXX

SEQ ID NO: 82 - protein, 14 aa, Escherichia coli
RRSAKAINFGLIYG

SEQ ID NO: 83 - protein, 14 aa, Thermus aquaticus
RRAAKTINFGVLYG

SEQ ID NO: 84 - protein, 14 aa, Artificial Sequence
  Consensus sequence
RXXGKXXNFGVLYG

SEQ ID NO: 85 - protein, 14 aa, Artificial Sequence
  Consensus sequence
RXXXKSANXGXXYG